(19) United States
(12) Patent Application Publication
(10) Pub. No.: US 2024/0299312 A1
(43) Pub. Date: Sep. 12, 2024

(54) LIPID NANOPARTICLE COMPOSITIONS AND METHODS OF USE THEREOF

(71) Applicant: Cureport Inc., Shrewsbury, MA (US)

(72) Inventor: De-Min Zhu, Shrewsbury, MA (US)

(21) Appl. No.: 18/023,564

(22) PCT Filed: Feb. 24, 2023
(86) PCT No.: PCT/US2023/013854

§ 371 Date: Feb. 27, 2023

Related U.S. Application Data
(60) Provisional application No. 63/313,967, filed on Feb. 25, 2022.

(51) Int. Cl. A61K 9/51; A61K 31/7105; A61K 31/711; C12N 15/88

(52) U.S. Cl. CPC A61K 9/5146; A61K 9/5123; A61K 9/5192; A61K 31/7105; A61K 31/711; C12N 15/88

(57) ABSTRACT

Disclosed herein are nanoparticle compositions including a polymer component and a lipid component. The disclosure also relates to lipid nanoparticles (LNPs) having the nanoparticle composition for nucleic acid delivery, and methods of making and using the same. The LNPs can be used for fast, efficient and safe delivery of nucleic acid (e.g., DNA/RNA).

### CLAIM OF PRIORITY

This application claims the benefit of U.S. Provisional Application App. No. 63/313,967, filed on Feb. 25, 2022. The entire contents of the foregoing application are incorporated herein by reference.

### TECHNICAL FIELD

This disclosure relates to nanoparticle compositions including a polymer component and a lipid component. The disclosure also relates to lipid nanoparticles (LNPs) having the nanoparticle composition for nucleic acid delivery, and methods of making and using the same.

### BACKGROUND

The tremendous successes in messenger RNA (mRNA)-based COVID-19 vaccines have emerged mRNA as well as plasmid DNA as new categories of therapeutic agents to prevent and treat various diseases. To function in vivo, mRNA requires safe, effective and stable delivery systems that protect the nucleic acid from degradation and that allow cellular uptake and mRNA release. Lipid nanoparticles (LNPs) have successfully entered the clinic for the delivery of mRNA; in particular, lipid nanoparticle-mRNA vaccines are now in clinical use against coronavirus disease 2019 (COVID-19), which marks a milestone for mRNA therapeutics. Clearly, LNP plays an essential role in mRNA delivery. For example, the LNP of the COVID-19 vaccines is made of 50 mol % ionizable lipid, 10 mol % DSPC, 38.5 mol % cholesterol, and 1.5 mol % PEG2000-DMG. In these components, the ionizable lipid plays two critical roles in the LNP: 1) encapsulating mRNA through the electrostatic interaction between its positive charged amino group with the negatively charged phosphate groups in the backbone of mRNA molecules; and 2) disrupting the membranes of endosome/lysosome for LNP escaping from the lysosomes and delivering mRNA into the cytosol of the cells. DSPC is a helper lipid to stabilize LNP; cholesterol is the structural lipid providing rigidity and stability to the LNP; and the PEGylated lipid PEG2000-DMG forms a PEG shell at the surface of LNP, preventing the particles from aggregation. Besides the lipid to lipid ratios between different lipids molecules, another critical parameter is the ratio between the ionizable lipid and mRNA encapsulated in the particles which is expressed as the N/P ratio. Here, N is the number of the nitrogen atoms of the positively ionizable amino groups of the ionizable lipid, and P is the number of the negatively charged phosphate groups of mRNA. The N/P ratio of the COVID-19 vaccine LNP formulation is 6:1, indicating the excessive component of ionizable lipid from the charge-charge ratio point of view.

Currently, this composition ratios (mol %) of 50% ionizable (or cationic) lipid, 10% DSPC, 38.5% cholesterol, and 1.5% of PEG2000-conjugated lipid represents the gold standard of LNP formulations. For example, recent publications reporting innovative ionizable lipids commonly use the above composition. See Sabnis, S., et al. “A novel amino lipid series for mRNA delivery: improved endosomal escape and sustained pharmacology and safety in non-human primates.” Molecular Therapy 26.6 (2018): 1509-1519; Li, S., et al. “Payload distribution and capacity of mRNA lipid nanoparticles.” Nature Communications 13.1 (2022): 5561; Herrera-Barrera, M., et al. “Peptide-guided lipid nanoparticles deliver mRNA to the neural retina of rodents and nonhuman primates.” Science Advances 9.2 (2023): eadd4623; and Chen, J., et al. “Lipid nanoparticle-mediated lymph node-targeting delivery of mRNA cancer vaccine elicits robust CD8+ T cell response.” Proceedings of the National Academy of Sciences 119.34 (2022): e2207841119.

However, the immunogenic ionizable lipid can cause the adverse reactions in the patients, from mild to life-threatening symptoms. See Szebeni, J., et al. “Applying lessons learned from nanomedicines to understand rare hypersensitivity reactions to mRNA-based SARS-CoV-2 vaccines.” Nature Nanotechnology 17.4 (2022): 337-346; Oster, M. E., et al. “Myocarditis cases reported after mRNA-based COVID-19 vaccination in the US from December 2020 to August 2021.” JAMA 327.4 (2022): 331-340. The toxicity of LNP badly limits its application in genetic vaccines and genetic therapeutics, particularly for the therapeutics that require higher dose of LNP with repetitive dosing regimens.

Thus, there is a need to develop next generation LNPs that can improve DNA/RNA delivery efficiency without cytotoxicity.

### SUMMARY

The present disclosure provides compositions and methods involving Polymer-Advanced Lipid Nanoparticles (PALNPs) to deliver nucleic acids (e.g., DNA or RNA) to cells.

In comparison to currently FDA-approved LNPs, the PALNPs disclosed herein demonstrated one or more, or all of the following superior features: i) plasmid DNA and mRNA delivery; ii) 10-50 folds of reduction in mRNA dose; iii) about 100-300 folds of reduction in the dose of cationic (ionizable) lipid; iv) over 95% success rate of cell transfection; v) 10-60 folds of higher protein expression; vi) fast cell uptake and protein expression; vii) non-cytotoxicity; and viii) long-lasting gene propagation and protein expression. Combining all the significantly improved key factors of transfection, including the low mRNA dose, robust cell transfection rate, and high protein expression, the mRNA/plasmid DNA delivery power of the PALNPs increase hundreds of times compared to the currently FDA-approved control LNPs.

In one aspect, the disclosure is related to a nanoparticle composition comprising a lipid component and a polymer component, in some embodiments, the polymer component comprises:
(a) a compound of formula I (PEOₓ-PPO_{y}-PEO_{z})

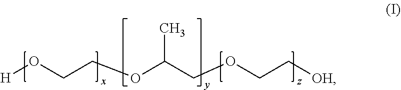

in some embodiments, x is a number selected from 1-100, y is a number selected from 10-500, and z is a number selected from 1-100;
(b) a compound of formula II (PPOₓ-PEO_{y}-PPO_{z})

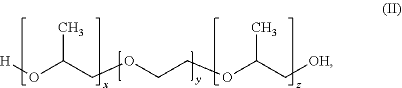

in some embodiments, x is a number selected from 10-500, y is a number selected from 1-100, and z is a number selected from 10-500;
(c) a compound of formula III (PEO_{y}-PPO_{z})

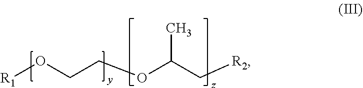

in some embodiments, R1 is H or CH₃, R2 is OH or OCH₃, y is a number selected from 1-100, and z is a number selected from 5-500; and/or
(d) a compound of formula IV (PPOₓ)

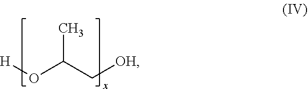

in some embodiments, x is a number selected from 1-100.

In some embodiments, the polymer component accounts for about 0.1 mol % to about 20 mol % of the nanoparticle composition.

In some embodiments, the lipid component comprises an ionizable and/or permanently charged cationic lipid, a helper lipid, a structural lipid, and/or a PEG (polyethylene glycol) lipid. In some embodiments, the ionizable and/or permanently charged cationic lipid accounts for about 5 mol % to about 30 mol % of the nanoparticle composition. In some embodiments, the helper lipid is a phospholipid. In some embodiments, the helper lipid accounts for about 10 mol % to about 50 mol % of the nanoparticle composition. In some embodiments, the structural lipid is cholesterol. In some embodiments, the structural lipid accounts for about 20 mol % to about 50 mol % of the nanoparticle composition. In some embodiments, the PEG lipid has an average molecular weight of about 500-5000 Daltons (e.g., about 2000 Daltons). In some embodiments, the PEG lipid accounts for about 0.5 mol % to about 5 mol % of the nanoparticle composition.

In some embodiments, the nanoparticle composition described herein comprises: (a) about 1 mol % to about 20 mol % of the compound of formula I; (b) about 5 mol % to about 30 mol % of the ionizable and/or permanently charged cationic lipid (e.g., ALC-0315, SM-102, DLin-DMA, DLin-MC3-DMA, and/or DLin-KC2-DMA); (c) about 10 mol % to about 50 mol % of the helper lipid (e.g., DSPC and/or DOPE); (d) about 20 mol % to about 50 mol % of the structural lipid (e.g., cholesterol); and (e) about 0.5 mol % to about 5 mol % of the PEG lipid (e.g., PEG2000-DSPE). In some embodiments, the number x and number z are identical in formula I. In some embodiments, the helper lipid comprises about 5 mol % to about 30 mol % of DSPC, and/or about 5 mol % to about 30 mol % of DOPE. In some embodiments, the compound of formula I has an average molecular weight of about 1000 Daltons to about 30000 Daltons (e.g., about 1000 Daltons to about 10000 Daltons). In some embodiments, the number x is selected from 1-15, the number y is selected from 30-80, and the number z is selected from 1-15. In some embodiments, the compound of formula I is L121, L92, or L81.

In some embodiments, the nanoparticle composition described herein comprises: (a) about 1 mol % to about 20 mol % of the compound of formula II; (b) about 5 mol % to about mol % of the ionizable and/or permanently charged cationic lipid (e.g., ALC-0315, SM-102, DLin-DMA, DLin-MC3-DMA, and/or DLin-KC2-DMA); (c) about 10 mol % to about 50 mol % of the helper lipid (e.g., DSPC and/or DOPE); (d) about 20 mol % to about 50 mol % of the structural lipid (e.g., cholesterol); and (e) about 0.5 mol % to about 5 mol % of the PEG lipid (e.g., PEG2000-DSPE). In some embodiments, the number x and number z are identical in formula II. In some embodiments, the helper lipid comprises about 5 mol % to about 30 mol % of DSPC, and/or about 5 mol % to about 30 mol % of DOPE. In some embodiments, the compound of formula II has an average molecular weight of about 1000 Daltons to about 30000 Daltons (e.g., about 1000 Daltons to about 10000 Daltons). In some embodiments, the number x is selected from 10-50, the number y is selected from 1-30, and the number z is selected from 10-50. In some embodiments, the compound of formula II is L31R1 or L17R4.

In some embodiments, the nanoparticle composition described herein comprises: (a) about 1 mol % to about 20 mol % of the compound of formula IV; (b) about 5 mol % to about mol % of the ionizable and/or permanently charged cationic lipid (e.g., ALC-0315, SM-102, DLin-DMA, DLin-MC3-DMA, and/or DLin-KC2-DMA); (c) about 10 mol % to about 50 mol % of the helper lipid (e.g., DSPC and/or DOPE); (d) about 20 mol % to about 50 mol % of the structural lipid (e.g., cholesterol); and (e) about 0.5 mol % to about 5 mol % of the PEG lipid (e.g., PEG2000-DSPE). In some embodiments, the helper lipid comprises about 5 mol % to about 30 mol % of DSPC, and/or about 5 mol % to about 30 mol % of DOPE. In some embodiments, the compound of formula IV has an average molecular weight of about 1000 Daltons to about 30000 Daltons (e.g., about 1000 Daltons to about 10000 Daltons). In some embodiments, number x in the compound of formula IV is about 30 to about 60. In some embodiments, the compound of formula IV is PPO₂₇₀₀.

In one aspect, the disclosure is related to a lipid nanoparticle (LNP) having the nanoparticle composition as described herein. In some embodiments, the LNP described herein further comprises a nucleic acid. In some embodiments, the nucleic acid comprises a DNA (e.g., double stranded DNA (dsDNA), plasmid DNA, single stranded DNA (ssDNA), or an antisense DNA thereof) or an RNA (e.g., small interfering RNA (siRNA), microRNA (miRNA), messenger mRNA (mRNA), guide RNA (gRNA), circular RNA (circRNA), self-amplifying RNA (saRNA), or an antisense RNA thereof).

In some embodiments, the N/P ratio of the nanoparticle composition is from about 0.1 to about 10 (e.g., about 0.1 to about 5, about 0.1 to about 4, about 0.1 to about 3, about 0.2 to about 2, or about 0.2 to about 1.5).

In some embodiments, the compound of formula I, formula II, formula III, formula IV, and/or formula V has a hydrophilic lipophilic balance (HLB) value from 1 to 18. In some embodiments, the mean size of the LNP is from about 30 nm to about 2000 nm (e.g., about nm to about 1000 nm, or about 30 nm to about 500 nm). In some embodiments, the polydispersity index of the LNP is from about 0.001 to about 0.5 (e.g., from about 0.01 to about 0.3). In some embodiments, the LNP has a zeta potential of about −30 mV to about +20 mV. In some embodiments, the w/w ratio of the lipid component to the nucleic acid is from about 2:1 to about 50:1 (e.g., from about 2:1 to about 20:1). In some embodiments, the encapsulation efficiency of the nucleic acid is at least 50%, at least 60%, at least 70%, at least 80%, or at least 90%.

In one aspect, the disclosure is related to a method of delivering a nucleic acid to a mammalian cell, in some embodiments, the method comprises administering the LNP as described herein to a subject, in some embodiments, the administering comprises contacting the mammalian cell with the nanoparticle composition, whereby the nucleic acid is delivered to the mammalian cell. In some embodiments, the mammalian cell is in a mammal. In some embodiments, the LNP is associated with a therapeutic medicine, a vaccine (e.g., a prophylactic vaccine or a therapeutic vaccine), gene editing, or cell-based therapies (e.g., chimeric antigen receptor (CAR)-T therapies). In some embodiments, the LNP is associated with treatment of a disease (e.g., infectious disease, autoimmune disease, cancers, or genetic disorders). In some embodiments, the LNP is delivered by mouth, nasal, dermal, vein, topical, ophthalmic, and/or mucosal, intradermal, and intramuscular administration.

In one aspect, the disclosure is related to a method for the enhanced delivery of a nucleic acid to a target tissue, in some embodiments, the method comprises administering the LNP as described herein to a subject, in some embodiments, the administering comprises contacting the target tissue with the LNP, whereby the nucleic acid is delivered to the target tissue.

In one aspect, the disclosure is related to a method of producing a polypeptide of interest in a mammalian cell, said method comprising administering the LNP as described herein to a subject, in some embodiments, the nucleic acid encodes the polypeptide of interest, whereby the nucleic acid is capable of being translated in the mammalian cell to produce the polypeptide of interest.

In one aspect, the disclosure is related to a method of reducing the expression of a target polypeptide (or a protein) in a mammalian cell, said method comprising administering the LNP as described herein to a subject. In some embodiments, the LNP is loaded with one or more antisense polynucleotides (e.g., siRNAs) that can substantially complement with a sequence encoding the target polypeptide (or a protein).

In one aspect, the disclosure is related to a method of making a LNP having a nanoparticle composition comprising a lipid component and a polymer component, in some embodiments, the lipid component comprises: an ionizable and/or permanently charged cationic lipid, a helper lipid, a structural lipid, and a PEG (polyethylene glycol) lipid, in some embodiments, the polymer component comprises: a compound of formula I, formula II, formula III, and/or formula IV, in some embodiments, the method comprises: (a) introducing one or more streams of a lipid solution in a water-miscible organic solvent via a first set of one or more inlet ports connected to a mixing chamber, in some embodiments, the lipid solution comprises the lipid component and the polymer component, (b) introducing one or more streams of an aqueous solution via a second set of one or more inlet ports connected to the mixing chamber, (c) mixing the one or more streams of the lipid solution and the one or more streams of the aqueous solution in a mixing chamber to generated the LNP, and (d) recovering the LNP via one or more outlet ports connected to the mixing chamber. In some embodiments, the aqueous solution comprises a nucleic acid. In some embodiments, the angle between any of the first set of one or more inlet ports and any of the second set of one or more inlet ports is 0-180 degrees.

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean+/−10% of the recited value. For instance, a nanoparticle composition including a lipid component having about 40% of a given compound may include 30-50% of the compound.

As used herein, the term “delivering” means providing an entity to a destination. For example, delivering an mRNA to a subject may involve administering a nanoparticle composition including the mRNA to the subject (e.g., by an intravenous, intramuscular, intradermal, or subcutaneous route). Administration of a nanoparticle composition to a mammal or mammalian cell may involve contacting one or more cells with the nanoparticle composition.

As used herein, a “lipid component” is a component of a nanoparticle composition that includes one or more lipids. For example, the lipid component may include one or more ionizable and/or permanently charged cationic lipids, helper lipids, structural lipids, PEG lipids, or other lipids. As used herein, a “polymer component” is a component of a nanoparticle composition that include one or more polymers. The term “alkyl”, employed alone or in combination with other terms, refers to a saturated hydrocarbon group that may be straight-chained or branched. The term “Cₙ₋ₘ alkyl” refers to an alkyl group having n to m carbon atoms. An alkyl group formally corresponds to an alkane with one C—H bond replaced by the point of attachment of the alkyl group to the remainder of the compound. The alkyl group may be unsubstituted or substituted. The alkyl group can be a primary, secondary, or tertiary alkyl group substituted with one or more substituents (e.g., one or more halo, as defined herein; haloalkyl, as defined herein; alkoxy, as defined herein; hydroxyalkyl, as defined herein; amino, as defined herein; amido, as defined herein; cyano (e.g., a —CN group); nitro (e.g., an —NO₂ group); hydroxyl (e.g., an —OH group); carboxyl (e.g., a —CO₂H group); oxo (e.g., an ═O group); carboxyaldehyde (e.g., a —C(O)H group); and the like, as well as combinations thereof). In some embodiments, the unsubstituted alkyl group contains 1 to 6, 1 to 4, 1 to 3, or 1 to 2 carbon atoms. Examples of alkyl groups include, but are not limited to, chemical groups such as methyl, ethyl, n-propyl, isopropyl, n-butyl, tert-butyl, isobutyl, sec-butyl, higher homologs (e.g., such as 2-methyl-1-butyl, n-pentyl, 3-pentyl, n-hexyl, 1,2,2-trimethylpropyl), and the like. The term “alkylene”, as used herein, refers to a multivalent (e.g., bivalent) form of an alkyl group, as described herein. Exemplary alkylene groups include methylene, ethylene, propylene, butylene, etc. In some embodiments, the alkylene group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, or C₂₋₂₄ alkylene group. The alkylene group can be branched or unbranched. The alkylene group can also be substituted or unsubstituted. For example, the alkylene group can be substituted with one or more substituents, as described herein for alkyl.

The term “alkenyl”, employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The alkenyl group may be unsubstituted or substituted (e.g., with one or more substituents, as described herein for alkyl). The term “Cₙ₋ₘ alkenyl” refers to an alkenyl group having n to m carbons. In some embodiments, the unsubstituted alkenyl group contains 2 to 6, 2 to 4, or 2 to 3 carbon atoms. Examples of alkenyl groups include, but are not limited to, ethenyl, n-propenyl, isopropenyl, n-butenyl, sec-butenyl, and the like.

The term “alkynyl”, employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The alkynyl group may be unsubstituted or substituted (e.g., with one or more substituents, as described herein for alkyl). The term “Cₙ₋ₘ alkynyl” refers to an alkynyl group having n to m carbons. In some embodiments, the unsubstituted alkynyl group contains 2 to 6, 2 to 4, or 2 to 3 carbon atoms. Examples of alkynyl groups include, but are not limited to, ethynyl, propyn-1-yl, propyn-2-yl, and the like.

The term “alkoxy”, employed alone or in combination with other terms, refers to a group of formula —O-alkyl, wherein the alkyl group is as defined above. The alkoxy group may be unsubstituted or substituted (e.g., with one or more substituents, as described herein for alkyl). The term “Cₙ₋ₘ alkoxy” refers to an alkoxy group, the alkyl group of which has n to m carbons. Examples of alkoxy groups include methoxy, ethoxy, propoxy (e.g., n-propoxy and isopropoxy), t-butoxy, and the like. In some embodiments, the alkyl group for the unsubstituted alkoxy group has 1 to 6, 1 to 4, or 1 to 3 carbon atoms.

The term “amido”, as used herein, refers to a group of formula —C(O)NR^{N1}R^{N2}, wherein each of R^{N1} and R^{N2} is, independently, H or optionally substituted alkyl, or R^{N1} and R^{N2}, taken together with the nitrogen atom to which each are attached, form a heterocyclyl group.

The term “amino”, as used herein, refers to a group of formula —NR^{N1}R^{N2}, wherein each of R^{N1} and R^{N2} is, independently, H or optionally substituted alkyl, or R^{N1} and R^{N2}, taken together with the nitrogen atom to which each are attached, form a heterocyclyl group.

The terms “halo” or “halogen”, used alone or in combination with other terms, refers to fluoro, chloro, bromo, and/or iodo. In some embodiments, “halo” refers to a halogen atom selected from F, Cl, or Br.

The term “haloalkyl”, as used herein, refers to an alkyl group in which one or more of the hydrogen atoms have been replaced by a halogen atom. The term “Cₙ₋ₘ haloalkyl” refers to a Cₙ₋ₘ alkyl group having n to m carbon atoms and from at least one up to {2(n to m)+1} halogen atoms, which may either be the same or different. In some embodiments, the halogen atoms are fluoro atoms. In some embodiments, the haloalkyl group has 1 to 6, 1 to 4, or 1 to 3 carbon atoms. Examples of haloalkyl groups include CF₃, C₂F₅, CHF₂, CCl₃, CHCl₂, C₂Cl₅, and the like.

The term “haloalkoxy”, employed alone or in combination with other terms, refers to a group of formula —O-haloalkyl, wherein the haloalkyl group is as defined above. The term “Cₙ₋ₘ haloalkoxy” refers to a haloalkoxy group, the haloalkyl group of which has n to m carbons. Examples of haloalkoxy groups include trifluoromethoxy, difluoromethoxy, and the like. In some embodiments, the haloalkoxy group has 1 to 6, 1 to 4, or 1 to 3 carbon atoms. The term “hydroxyalkyl”, as used herein, refers to an alkyl group in which one or more of the hydrogen atoms have been replaced by a hydroxyl group (e.g., an —OH group). The term “Cₙ₋ₘ hydroxyalkyl” refers to a Cₙ₋ₘ alkyl group having n to m carbon atoms and from at least one up to {2(n to m)+1} hydroxyl groups. In some embodiments, the hydroxyalkyl group includes one to three hydroxyl groups. In some embodiments, the hydroxyalkyl group has 1 to 6, or 1 to 4, or 1 to 3 carbon atoms. Examples of hydroxyalkyl groups include hydroxymethyl, 1-hydroxyethyl, 2-hydroxyethyl, and the like.

The term, “salt” means an ionic form of a compound or structure (e.g., any formulas, compounds, or compositions described herein), which includes a cation or anion compound to form an electrically neutral compound or structure. Salts (e.g., simple salts having binary compounds, double salts, triple salts, etc.) are well known in the art. For example, salts are described in Berge S M et al., “Pharmaceutical salts,” J. Pharm. Sci. 1977 January; 66(1):1 19; International Union of Pure and Applied Chemistry, “Nomenclature of Inorganic Chemistry,” Butterworth & Co. (Publishers) Ltd., London, England, 1971 (2nd ed.); and in “Handbook of Pharmaceutical Salts: Properties, Selection, and Use,” Wiley VCH, April 2011 (2nd rev. ed., eds. P. H. Stahl and C. G. Wermuth). The salts can be prepared in situ during the final isolation and purification of the compounds or separately by reacting the free base group with a suitable organic acid (thereby producing an anionic salt) or by reacting the acid group with a suitable metal or organic salt (thereby producing a cationic salt). Representative anionic salts include acetate, adipate, alginate, ascorbate, aspartate, benzenesulfonate, benzoate, bicarbonate, bisulfate, bitartrate, borate, bromide, butyrate, camphorate, camphorsulfonate, chloride, citrate, cyclopentanepropionate, digluconate, dihydrochloride, diphosphate, dodecylsulfate, edetate, ethanesulfonate, fumarate, glucoheptonate, glucomate, glutamate, glycerophosphate, hemisulfate, heptonate, hexanoate, hydrobromide, hydrochloride, hydroiodide, hydroxyethanesulfonate, hydroxynaphthoate, iodide, lactate, lactobionate, laurate, lauryl sulfate, malate, maleate, malonate, mandelate, mesylate, methanesulfonate, methylbromide, methylnitrate, methylsulfate, mucate, 2 naphthalenesulfonate, nicotinate, nitrate, oleate, oxalate, palmitate, pamoate, pectinate, persulfate, 3 phenylpropionate, phosphate, picrate, pivalate, polygalacturonate, propionate, salicylate, stearate, subacetate, succinate, sulfate, tannate, tartrate, theophyllinate, thiocyanate, triethiodide, toluenesulfonate, undecanoate, valerate salts, and the like. Representative cationic salts include metal salts, such as alkali or alkaline earth salts, e.g., barium, calcium (e.g., calcium edetate), lithium, magnesium, potassium, sodium, and the like; other metal salts, such as aluminum, bismuth, iron, and zinc; as well as nontoxic ammonium, quaternary ammonium, and amine cations, including, but not limited to ammonium, tetramethylammonium, tetraethylammonium, methylamine, dimethylamine, trimethylamine, triethylamine, ethylamine, pyridinium, and the like. Other cationic salts include organic salts, such as chloroprocaine, choline, dibenzylethylenediamine, diethanolamine, ethylenediamine, methylglucamine, and procaine.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows the GFP fluorescence images in HEK293 cells. Upper row: cells transfected with 0.6 μg of GFP mRNA delivered by control LNPs (N/P=6) of the specified cationic lipids (cationic lipid 50%, DSPC 10%, cholesterol 38.5%, PEG2000-DSPE 1.5%). Lower row: 0.1 μg GFP mRNA delivered by PALNPs (N/P=6).

FIG. 2 shows a flow cytometry result of cells transfected by CPT108 with 0.05 μg GFP mRNA and cells transfected by CPT107 (“ACL-0315”) with 0.6 μg GFP mRNA (control).

FIG. 3 show the average fluorescence intensity and percentage of GFP-positive cells determined by FACS. The cells were transfected by CPT108 loaded with 50 ng, 100 ng, 200 ng, or 400 ng GFP mRNA. Cells transfected with CPT107 (“ACL-0315”) loaded with 600 ng GFP mRNA was used as a control.

FIG. 4 shows mRNA delivery enhancement curves. The overall enhancement was calculated from the products of the ratios (mRNA doses, cell transfection rates, and fluorescence intensities) between the PALNP and the corresponding control LNP.

FIGS. 5A-5C show GFP fluorescence images of HEK293 cells transfected with CPT147 LNP (FIG. 5A: 0.01 μg mRNA, N/P=1; FIG. 5B: 0.3 μg mRNA, N/P=1), or CPT107 LNP (FIG. 5C: 0.3 μg mRNA, N/P=6).

FIG. 6A shows a set of fluorescence images of cells transfected with a fixed quantity of CPT147 LNP loaded with GFP mRNA. The mRNA was 0.025-0.4 μg, and the corresponding N/P ratio ranged from 0.25 to 4.

FIG. 6B shows a set of fluorescence images of cells transfected with CPT147 LNP loaded with GFP mRNA. The mRNA was fixed at 0.05 μg, and the N/P ratio ranged from 0.25 to 4.

FIG. 6C shows a set of fluorescence images of cells transfected with CPT147-38 LNP loaded with GFP mRNA. The mRNA was fixed at 0.25 μg, and the N/P ratio ranged from 0.25 to 2.

FIG. 6D shows a set of fluorescence images of cells transfected with CPT163-21-18 LNP loaded with GFP mRNA. The mRNA was fixed at 0.25 μg, and the N/P ratio ranged from 0.25 to 2.

FIG. 7 shows a set of fluorescence images of cells transfected with CPT147 (N/P=1) for 1 hour, 2 hours, or 4 hours. The cells emitted red fluorescence originated from rhodamine-DSPC labeled LNP (Rhd-LNP) and green fluorescence originated from the expressed GFP protein (GFP).

FIG. 8A shows a fluorescence image of cells transfected by Rhodamine-DSPC labeled CPT147 2 hours post transfection.

FIG. 8B shows a fluorescence image of cells transfected by Rhodamine-DSPC labeled control LNP (CPT107) 2 hours post transfection.

FIG. 9A shows a fluorescence image of cells transfected with CPT147 LNP loaded with GFP mRNA for 1 hour and then cultured in a fresh medium for 3 hours. The CPT147 LNP were labeled with Rhodamine-DSPC.

FIG. 9B shows a fluorescence image of cells transfected with CPT147 LNP loaded with GFP mRNA for 4 hours. The CPT147 LNP were labeled with Rhodamine-DSPC.

FIG. 9C shows a schematic diagram of mRNA transfection in FIG. 9A (Group A) and FIG. 9B (Group B).

FIGS. 10A-10B show GFP fluorescent and bright field images of HEK293 cells after 96-hour transfection with CPT147 LNP (mRNA 0.05 μg, N/P=1).

FIGS. 11A-11B show fluorescence images of cells transfected with CPT147 loaded with 0.05 μg GFP plasmid DNA for 48 hours (FIG. 12A) and 120 hours (FIG. 12B).

FIG. 11C shows an enlarged bright field image of the cells in the rectangle of FIG. 12B overlapped with the corresponding GFP fluorescence image.

FIGS. 11D-11E show fluorescence images of cells split from the cells in FIG. 11B and cultured in a fresh medium for an additional 24 hours (FIG. 11D) or 144 hours (FIG. 11E). Cell colonies emitting green fluorescent signals are marked by circles.

FIGS. 11F-11G show fluorescence images of cells split from the cells in FIG. 11E and cultured in a fresh medium for an additional 24 hours (FIG. 11F) or 120 hours (FIG. 11G). Cell colonies emitting green fluorescent signals are marked by circles.

FIG. 12 shows a set of fluorescence images of cells transfected with CPT147E LNP loaded with 0.05 μg GFP mRNA (N/P=1; transfection time 24 hours). The LNP was stored at 25° C. for 0 hour (“TO”), 4 hours, 8 hours, 12 hours, 24 hours, 48 hours, 72 hours, 120 hours, or 168 hours before transfection.

FIG. 13 shows a set of fluorescence images of cells transfected with CPT147E LNP loaded with 0.05 μg, 0.01 μg, 0.005 μg, or 0.0025 μg GFP mRNA. The LNP was stored at 25° C. for 0 hour, 48 hours, 72 hours, 120 hours, or 168 hours before transfection.

FIGS. 14A-14F show a set of fluorescence images of cells transfected with CPT147E-05 (FIG. 14A), CPT147E_{control} (FIG. 14B), CPT149E_{control} (FIG. 14C), CPTI49E_{control} (FIG. 14D), CPT162E_{control} (FIG. 14E), or CPT62E_{control} (FIG. 14F). The LNPs were loaded with 0.2 μg, 0.1 μg, 0.05 μg, or 0.025 μg GFP mRNA.

FIG. 15 shows a set of fluorescence images of HEK-293 cells transfected by LNPs made of varies of ionizable lipids and PEOₓ-PPO_{y}-PEOₓ triblock co-polymers. The LNPs were loaded with 0.2 μg, 0.1 μg, or 0.05 μg GFP mRNA.

FIG. 16 shows fluorescence images of HEK-293 cells transfected by CPT153E LNP. The quantities of GFP mRNA (μg/well) are specified on top of the images.

FIG. 17 shows fluorescence images of HEK-293 cells transfected by CPT189 LNP (the top row) and CPT202 LNP (the bottom row). The quantities of GFP mRNA (μg/well) are specified on top of the images.

FIGS. 18A-18B show fluorescence images of HEK-293 cells transfected by the base LNPs plus different molar ratios (mol %) of co-polymer L81 or L92. The molar ratios of the polymer are specified on top of the images. The GFP mRNA added to each well was 0.25 μg. The images were taken 24 hours after the LNPs were added to the cells.

FIGS. 19A-19D show bright field images of cells transfected with CPT147P (FIG. 19A), CPT107 (FIG. 19B), CPT149E (FIG. 19C), or CPT109 (FIG. 19D) that contained 0.2 μg GFP mRNA for 24 hours.

FIGS. 20A-20B show body images of BALB/c mice injected with LNPs carrying luciferase reporter mRNA for 4 hours, 8 hours, 12 hours, or 24 hours. The PALNP CPT147E-10 and the corresponding reference LNP CPT107 containing 10 μg luciferase mRNA were injected via the tail veins of the mice.

FIG. 21 shows body images of BALB/c mice injected with LNP carrying luciferase reporter mRNA for 6 hours, 12 hours, or 24 hours. The PALNP CPT147E-10 and the corresponding reference LNP CPT107 containing 1 μg luciferase mRNA were injected via the tail veins of the mice.

FIG. 22 shows body images of BALB/c mice injected with LNP carrying luciferase reporter mRNA for 4 hours, 8 hours, 12 hours, 24 hours, 48 hours, or 72 hours. The PALNP CPT147E-10 and the corresponding reference LNP CPT107 containing 1 μg luciferase mRNA were injected via the tail veins of the mice.

FIG. 23 shows the liver and spleen targeting distribution of mRNA delivered by LNP CPT147E-10.

FIG. 24 shows body images of BALB/c mice injected with LNP CPT147 by intramuscular injection. The images were taken 4 hours, 8 hours, 12 hours, 24 hours, 36 hours, or 48 hours post injection.

FIG. 25 shows the T-mixing setup for generating LNPs.

FIG. 26 shows particle size distribution of LNP (size/polydispersity). The blank LNP (128.3 nm/0.220); mRNA LNP made by the blank LNP and mRNA (180.1 nm/0.125); and mRNA LNP made by directly mixing the lipids and mRNA (164.7 nm/0.075) are labeled with arrows.

FIG. 27 shows fluorescence images of HEK-293 cells transfected by CPT201 LNP. The quantities of GFP mRNA (μg/well) are specified on top of the images.

### DETAILED DESCRIPTION

The present disclosure provides a safe and efficient LNP (e.g., with up to 95%) deduction of ionizable lipid usage as compared to the standard LNPs. However, since the development of the first LNP, the molar ratio of the cationic/ionizable lipid has been kept at about 50 mol %, and the effective N/P ratios in the FDA-approved siRNA therapeutics and mRNA vaccines are 3-6. This is because the second role of the ionizable lipid to disrupt lysosome membrane for mRNA escaping requires a high quantity of ionizable lipid. A simple cut down to ionizable lipid can jeopardize the functions of LNP. Thus, the present disclosure aimed to identify a clinically demonstrated nonimmunogenic, nontoxic material for the LNP to enhance its cell uptake and promote mRNA delivery.

Poloxamer, also known as pluronic, is a class of nonionic surfactants that are widely used in pharmaceuticals, cosmetics, and various industrial applications. It is a copolymer composed of polyoxyethylene (PEO) and polyoxypropylene (PPO) blocks arranged in a specific structure. The ratio of these blocks and the length of the polyoxyethylene and polyoxypropylene chains determine the properties of the poloxamer. The general formula for poloxamer (pluronic) is:

HO—(CH₂—CH₂—O)ₙ—(CH₂—CH(CH₃)—O)ₘ—(CH₂—CH₂—O)ₙ—H

where n and m are numbers that represent the number of repeating units of the polyoxyethylene and polyoxypropylene blocks, respectively. The value of n and m can vary depending on the specific poloxamer product.

Generally the typical structure of poloxamer is triblock copolymer, either PEOₙ-PPOₘ-PEOₙ, or PPOₘ-PEOₙ-PPOₘ. While, the diblock copolymer of PEOₙ-PPOₘ is also classifies as poloxamer. See Alvarez-Lorenzo, C., A. Sosnik, and A. Concheiro. “PEO-PPO block copolymers for passive micellar targeting and overcoming multidrug resistance in cancer therapy.” Current Drug Targets 12.8 (2011): 1112-1130. As an extreme condition, when either m or n equals to 0, the polymer PEO, or PPO can also be referred to as a poloxamer.

In addition, poloxamer are non-toxic and non-irritant, making them attractive materials for biomedical applications. Furthermore, poloxamers have a proven record of FDA approval and are listed in the US and European Pharmacopeia for uses as stabilizers, emulsifiers, solubilizers, and for topical, parenteral, and oral administration. See Russo, E, et al., “Poloxamer hydrogels for biomedical applications.” Pharmaceutics 11.12 (2019): 671.

In the present disclosure, poloxamer is incorporated into lipid nanoparticles to form a new class of Poloxamer-Advanced Lipid Nanoparticle (PALNP). In PALNP, the N/P ratio is reduced from the standard 6 to the range of about 1-0.25 (about 6-24 folds deduction), and the weight percentage of mRNA to the total lipids is increased from 4% of the standard to about 10-20%. The in vitro and in vivo gene delivery efficiency is dramatically increased, without observation of cytotoxicity.

In some embodiments, this disclosure relates to nanoparticle compositions including a lipid component and a polymer component, and methods of using the same. In some embodiments, the disclosure provides methods of producing a polypeptide of interest in a cell that involves contacting a nucleic acid-loaded LNP (e.g., any of the LNPs described herein) with a mammalian cell, whereby the nucleic acid may be translated to produce the polypeptide of interest. The disclosure further includes methods of delivering a nucleic acid to a mammalian cell involving administration of a nucleic acid-loaded LNP (e.g., any of the LNPs described herein) to a subject, in which the administration involves contacting a cell with the LNP, whereby the nucleic acid is delivered to a cell.

In some embodiments, the LNPs (e.g., PALNPs) or LNPs having the nanoparticle compositions described herein have a N/P ratio that is less than 90%, less than 80%, less than 70%, less than 60%, less than 50%, less than 40%, less than 30%, less than 20%, or less than 10% as compared to a reference LNP that does not include the polymer component (e.g., any of the polymer components described herein), except the polyethylene glyco-conjugated lipid, e.g., LNPs used as COVID-10 vaccines described herein.

In some embodiments, the LNPs (e.g., PALNPs) or LNPs having the nanoparticle compositions described herein is more stable than a reference LNP that does not include the polymer component (e.g., any of the polymer components described herein), e.g., LNPs used as COVID-10 vaccines described herein. For example, the LNPs (e.g., PALNPs) can be stored at 25° C. for at least a week without substantially affecting its transfection efficiency in cells.

In some embodiments, the LNPs (e.g., PALNPs) or LNPs having the nanoparticle compositions described herein are effective for both DNA and RNA transfections. In some embodiments, the cell transfection efficiency of the LNPs (e.g., PALNPs) is increased by at least 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold as compared to a reference LNP loaded with the same amount of cargo molecules (e.g., any of the nucleic acids described herein), but does not include the polymer component (e.g., any of the polymer components described herein), e.g., LNPs used as COVID-10 vaccines described herein. In some embodiments, the cell transfection efficiency is determined by measuring fluorescence emitted from transfected cells.

In some embodiments, the LNPs (e.g., PALNPs) or LNPs having the nanoparticle compositions described herein can promote fast cell uptake and/or protein expression. For example, after transfection using the LNPs (e.g., PALNPs) loaded with a nucleic acid encoding a protein of interest, the transfected cells can express the protein of interest within 1 hour, 2 hours, 3 hours, 4 hours, 5 hours, 6 hours, 7 hours, 8 hours, 9 hours, 10 hours, 11 hours, 12 hours, or 24 hours. In some embodiments, the transfection can be carried out for less than 2 hours, less than 1.5 hours, less than 1 hours, less than 55 minutes, less than 50 minutes, less than 45 minutes, less than 40 minutes, less than 35 minutes, less than 30 minutes, less than 25 minutes, less than 20 minutes, less than 15 minutes, less than 10 minutes, or less than 5 minutes. In some embodiments, the LNPs (e.g., PALNPs) or LNPs having the nanoparticle composition described herein can be administered to a subject (e.g., by intramuscular administration), without entering into the systemic delivery of the subject.

In some embodiments, the LNPs (e.g., PALNPs) or LNPs having the nanoparticle compositions described herein can increase the protein expression level from the same dose of a cargo molecule (e.g., any of the nucleic acids described herein) by at least 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, or 100-fold as compared to a reference LNP, e.g., a LNP that does not include the polymer component (e.g., any of the polymer components described herein), e.g., LNPs used as COVID-10 vaccines described herein.

In some embodiments, provided herein are methods of delivering a cargo molecule (e.g., any of the nucleic acids described herein) via the LNPs described herein or LNPs having the nanoparticle compositions described herein. The methods can use less than 50%, less than 40%, less than 30%, less than 20%, less than 10%, less than 5%, less than 4%, less than 3%, less than 2%, or less than 1% of the dose of the cargo molecule (e.g., any of the nucleic acids described herein) to achieve a comparable transfection efficiency when a reference LNP, e.g., a LNP that does not include the polymer component (e.g., any of the polymer components described herein), e.g., LNPs used as COVID-10 vaccines described herein.

In some embodiments, the success rate of cell transfection using the LNPs (e.g., PALNPs) or LNPs having the nanoparticle compositions described herein is at least 90%, 91%, 92%, 93%, 94%, 95%, 95%, 97%, 98%, or 99% using less than 1000 ng, 500 ng, 200 ng, 100 ng, 50 ng, 40 ng, 30 ng, 20 ng, 10 ng, 5 ng, or 1 ng.

### Polymer-Advanced Lipid Nanoparticles (PALNPs)

In some embodiments, the nanoparticle composition described herein is a PALNP. In some embodiments, the nanoparticle composition described herein includes a lipid component (e.g., any of the lipid components described herein), a polymer component (e.g., any of the polymer components described herein). In some embodiments, the nanoparticle composition also includes a cargo molecule (e.g., any of the nucleic acids described herein).

### Polymer Component

In some embodiments, the polymer component described herein includes any of the compounds described herein or combinations thereof. In some embodiments, the compounds described herein is derived from Formula I, Formula II, Formula III, or Formula IV.

### Formula I (Triblock Co-Polymer)

In some embodiments, the polymer component described herein includes a triblock co-polymer. In some embodiments, the triblock co-polymer is a Pluronic™ surfactant. In some embodiments, the tri-block co-polymer is a poloxamer. Specifically, poloxamers are nonionic triblock co-polymers including a central hydrophobic chain of polyoxypropylene (poly(propylene oxide)) flanked by two hydrophilic chains of polyoxyethylene (poly(ethylene oxide)).

In some embodiments, the triblock co-polymer is derived from a compound of formula I with a general structure shown below:

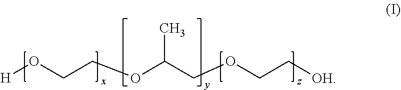

In some embodiments, formula I is also depicted as PEOₓ-PPO_{y}-PEO_{z}, [PEO]ₓ—[PPO]_{y}—[PEO]_{z}, PEO-PPO-PEO, poly(ethylene oxide)-poly(propylene oxide)-poly(ethylene oxide), PEG-PPG-PEG, poly(ethylene glycol)-poly(propylene glycol)-poly(ethylene glycol), or H[OCH₂CH₂]ₓ[OCH(CH₃)CH₂]_{y}[OCH₂CH₂]_{z}OH. In some embodiments, x, y, and z in formula I or its equivalent formula thereof are numbers. For example, x is a number selected from 1-100, y is a number selected from 10-500, and z is a number selected from 1-100. In some embodiments, the numbers x and z are identical. In some embodiments, the number y is at least 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold higher than numbers x and/or z.

In some embodiments, the triblock co-polymer is derived from a compound of formula I′ with a general structure shown below:

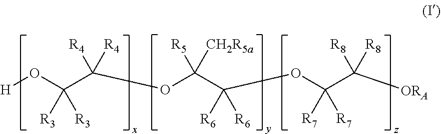

or a salt thereof,
wherein:
each R₃, R₄, R₅, R₅ₐ, R₆, R₇, and R₈ is, independently, selected from the group consisting of hydrogen (H), C₁₋₆ alkyl, C₂₋₆ alkenyl, C₂₋₆ alkynyl, C₁₋₆ alkoxy, C₁₋₆ haloalkyl, C₁₋₆haloalkoxy, C₁₋₆ hydroxyalkyl, halo, cyano (—CN), nitro (—NO₂), hydroxyl (—OH), carboxyl (—COOH), carboxyaldehyde (—C(O)H), amido (e.g., —C(O)NH₂, —C(O)NHC₁₋₄ alkyl, or —C(O)N(C₁₋₄ alkyl)₂), and amino (e.g., —NH₂, —NHC₁₋₄ alkyl, or —N(C₁₋₄ alkyl)₂; and
R_{A} is selected from the group consisting of hydrogen (H), C₁₋₆ alkyl, C₂₋₆ alkenyl, C₂₋₆ alkynyl, C₁₋₆ haloalkyl, and C₁₋₆ hydroxyalkyl.

In some embodiments, each R₃, R₄, R₅, R₅ₐ, R₆, R₇, R₈, and R_{A} is H.

In some embodiments, each R₃, R₄, R₅, R₅ₐ, R₆, R₇, is R₈ is H; and R_{A} is methyl.

In some embodiments, each R₃ is H. In some embodiments, at least one R₃ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, at least one R₃ is selected from methyl, ethyl, methoxy, ethoxy, hydroxymethyl, halo, and hydroxyl. In some embodiments, at least one R₃ is methyl. In some embodiments, at least one R₃ is ethyl. In some embodiments, at least one R₃ is methoxy. In some embodiments, at least one R₃ is ethoxy. In some embodiments, at least one R₃ is hydroxymethyl. In some embodiments, at least one R₃ is halo. In some embodiments, at least one R₃ is hydroxyl.

In some embodiments, each R₄ is H. In some embodiments, at least one R₄ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, at least one R₄ is selected from methyl, ethyl, methoxy, ethoxy, hydroxymethyl, halo, and hydroxyl. In some embodiments, at least one R₄ is methyl. In some embodiments, at least one R₄ is ethyl. In some embodiments, at least one R₄ is methoxy. In some embodiments, at least one R₄ is ethoxy. In some embodiments, at least one R₄ is hydroxymethyl. In some embodiments, at least one R₄ is halo. In some embodiments, at least one R₄ is hydroxyl.

In some embodiments, R₅ is H. In some embodiments, R₅ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, R₅ is selected from methyl, ethyl, methoxy, ethoxy, halo, and hydroxyl. In some embodiments, R₅ is methyl. In some embodiments, R₅ is ethyl. In some embodiments, R₅ is methoxy. In some embodiments, R₅ is ethoxy. In some embodiments, R₅ is hydroxymethyl. In some embodiments, R₅ is halo. In some embodiments, R₅ is hydroxyl.

In some embodiments, R₅ₐ is H. In some embodiments, R₅ₐ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, Ria is selected from methyl, ethyl, methoxy, ethoxy, halo, and hydroxyl. In some embodiments, R₅ₐ is methyl. In some embodiments, R₅ₐ is ethyl. In some embodiments, R₅, is methoxy. In some embodiments, R₅ₐ is ethoxy. In some embodiments, R₅ₐ is hydroxymethyl. In some embodiments, Ria is halo. In some embodiments, Ria is hydroxyl.

In some embodiments, each R₆ is H. In some embodiments, at least one R₆ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, at least one R₆ is selected from methyl, ethyl, methoxy, ethoxy, halo, and hydroxyl. In some embodiments, at least one R₆ is methyl. In some embodiments, at least one R₆ is ethyl. In some embodiments, at least one R₆ is methoxy. In some embodiments, at least one R₆ is ethoxy. In some embodiments, at least one R₆ is hydroxymethyl. In some embodiments, at least one R₆ is halo. In some embodiments, at least one R₆ is hydroxyl.

In some embodiments, each R₇ is H. In some embodiments, at least one R₇ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, at least one R₇ is selected from methyl, ethyl, methoxy, ethoxy, halo, and hydroxyl. In some embodiments, at least one R₇ is methyl. In some embodiments, at least one R₇ is ethyl. In some embodiments, at least one R₇ is methoxy. In some embodiments, at least one R₇ is ethoxy. In some embodiments, at least one R₇ is hydroxymethyl. In some embodiments, at least one R₇ is halo. In some embodiments, at least one R₇ is hydroxyl.

In some embodiments, each R₈ is H. In some embodiments, at least one R₈ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, at least one R₈ is selected from methyl, ethyl, methoxy, ethoxy, halo, and hydroxyl. In some embodiments, at least one R₈ is methyl. In some embodiments, at least one R₈ is ethyl. In some embodiments, at least one R₈ is methoxy. In some embodiments, at least one R₈ is ethoxy. In some embodiments, at least one R₈ is hydroxymethyl. In some embodiments, at least one R₈ is halo.

In some embodiments, R_{A} is H. In some embodiments, R_{A} is selected from C₁₋₆ alkyl, C₂₋₆ alkenyl, C₂₋₆ alkynyl, C₁₋₆ haloalkyl, and C₁₋₆ hydroxyalkyl. In some embodiments, R_{A} is selected from methyl and ethyl. In some embodiments, R_{A} is methyl. In some embodiments, R_{A} is ethyl. In some embodiments, R_{A} is hydroxymethyl.

In some embodiments, at least one R_{A} is hydroxyl. In some embodiments, the triblock co-polymer is derived from a compound of formula I″ with a general structure shown below:

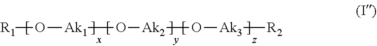

or a salt thereof,
wherein:
R₁ is hydrogen (H), optionally substituted alkyl (e.g., C₁₋₆, C₁₋₄, C₁₋₃, or C₁₋₂ alkyl), or optionally substituted hydroxyalkyl (e.g., C₁₋₆, C₁₋₄, C₁₋₃, or C₁₋₂ hydroxyalkyl);
R₂ is hydroxyl (—OH), optionally substituted alkoxy (e.g., C₁₋₆, C₁₋₄, C₁₋₃, or C₁₋₂ alkoxy), or optionally substituted hydroxyalkyl (e.g., C₁₋₆, C₁₋₄, C₁₋₃, or C₁₋₂ hydroxyalkyl); each of Ak₁, Ak₂, and Ak₃ is, independently, an optionally substituted alkylene (e.g., optionally substituted C₁₋₆, C₁₋₄, C₁₋₃, or C₁₋₂ alkylene);
each of Ak₁, Ak₂, and Ak₃ is not identical; and
x, y, and z can be any described herein (e.g., as described herein for formula I).

In some embodiments, R₁ is hydrogen or C₁₋₆ alkyl. In some embodiments, R₂ is hydroxyl or C₁₋₆ alkoxy. In some embodiments, R₁ is hydrogen, and R₂ is hydroxyl. In some embodiments, R₁ is hydrogen, and R₂ is C₁₋₆ alkoxy. In some embodiments, R₁ is C₁₋₆ alkyl, and R₂ is hydroxyl. In some embodiments, R₁ is C₁₋₆ alkyl, and R₂ is C₁₋₆ alkoxy.

In some embodiments, Ak₁ and Ak₃ are the same, but Ak₂ is different.

In some embodiments, the optionally substituted alkylene is substituted with one or more substituents. Non-limiting examples of substituents include one or more of the following: halo, haloalkyl, alkoxy, hydroxyalkyl, amino, amido, cyano, nitro, hydroxyl, carboxyl, oxo, carboxyaldehyde, or a combination thereof.

In some embodiments, at least one of Ak₁, Ak₂, and Ak₃ is, independently, an optionally substituted C₁₋₆, C₁₋₄, C₁₋₃, or C₁₋₂ alkylene. In some embodiments, each of Ak₁, Ak₂, and Ak₃ is, independently, an optionally substituted C₁₋₆, C₁₋₄, C₁₋₃, or C₁₋₂ alkylene.

In some embodiments, at least one of Ak₁, Ak₂, and Ak₃ is, independently, an unsubstituted C₁₋₆, C₁₋₄, C₁₋₃, or C₁₋₂ alkylene. In some embodiments, each of Ak₁, Ak₂, and Ak₃ is, independently, an unsubstituted C₁₋₆, C₁₋₄, C₁₋₃, or C₁₋₂ alkylene.

In some embodiments, at least one of Ak₁, Ak₂, and Ak₃ is, independently, an unbranched alkylene (e.g., an unbranched C₁₋₆, C₁₋₄, C₁₋₃, or C₁₋₂ alkylene) that may be optionally substituted. In some embodiments, at least two of Ak₁, Ak₂, and Ak₃ is, independently, an unbranched alkylene. In some embodiments, each of Ak₁ and Ak₃ is, independently, an unbranched alkylene.

In some embodiments, at least one of Ak₁, Ak₂, and Ak₃ is a branched alkylene (e.g., a branched C₂₋₆, C₂₋₄, C₃₋₆, or C₃₋₄ alkylene) that may be optionally substituted. In some embodiments, Ak₂ is a branched alkylene.

In some embodiments, the number x is about 1-100, 1-90, 1-80, 1-70, 1-60, 1-50, 1-40, 1-30, 1-20, 1-10, 1-5, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100; the number y is about 10-500, 10-450, 10-400, 10-350, 10-300, 10-250, 10-200, 10-150, 10-100, 10-90, 10-80, 10-70, 10-60, 10-50, 10-40, 10-30, 10-20, 20-500, 20-450, 20-400, 20-350, 20-300, 20-250, 20-200, 20-150, 20-100, 20-90, 20-80, 20-70, 20-60, 20-50, 20-40, 20-30, 30-500, 30-450, 30-400, 30-350, 30-300, 30-250, 30-200, 30-150, 30-100, 30-90, 30-80, 30-70, 30-60, 30-50, 30-40, 40-500, 40-450, 40-400, 40-350, 40-300, 40-250, 40-200, 40-150, 40-100, 40-90, 40-80, 40-70, 40-60, 40-50, 50-500, 50-450, 50-400, 50-350, 50-300, 50-250, 50-200, 50-150, 50-100, 50-90, 50-80, 50-70, 50-60, 60-500, 60-450, 60-400, 60-350, 60-300, 60-250, 60-200, 60-150, 60-100, 60-90, 60-80, 60-70, 70-500, 70-450, 70-400, 70-350, 70-300, 70-250, 70-200, 70-150, 70-100, 70-90, 70-80, 80-500, 80-450, 80-400, 80-350, 80-300, 80-250, 80-200, 80-150, 80-100, 80-90, 90-500, 90-450, 90-400, 90-350, 90-300, 90-250, 90-200, 90-150, 90-100, 100-500, 100-450, 100-400, 100-350, 100-300, 100-250, 100-200, 100-150, 150-500, 150-450, 150-400, 150-350, 150-300, 150-250, 150-200, 200-500, 200-450, 200-400, 200-350, 200-300, 200-250, 250-500, 250-450, 250-400, 250-350, 250-300, 300-500, 300-450, 300-400, 300-350, 350-500, 350-450, 350-400, 400-500, 400-450, or 450-500; and the number z is about 1-100, 1-90, 1-80, 1-70, 1-60, 1-50, 1-40, 1-30, 1-20, 1-10, 1-5, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100. In some embodiments, the number x is selected from 1-15, the number y is selected from 30-80, and the number z is selected from 1-15.

In some embodiments, the number x is about 1, 2, 3, 4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 7, 8, 9, or 10 (e.g., about 5 or about 6), the number y is about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 (e.g., about 67 or about 68), and the number z is about 1, 2, 3, 4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 7, 8, 9, or 10 (e.g., about 5 or about 6). In some embodiments, the number x is about 1, 2, 3, 4, 5, 6, 7, 8, 9, 10, 11, 12, 13, 14, 15, 16, 17, 18, 19, or 20 (e.g., about 10), the number y is about 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 (e.g., about 47), and the number z is about 1, 2, 3, 4, 5, 6, 7, 8, 9, 10, 11, 12, 13, 14, 15, 16, 17, 18, 19, or 20 (e.g., about 10). In some embodiments, the number x is about 1, 2, 3, 4, 5, 6, 7, 7.5, 7.6, 7.7, 7.8, 7.9, 8, 8.1, 8.2, 8.3, 8.4, 8.5, 8.6, 8.7, 8.8, 8.9, 9, 10, 11, 12, 13, 14, or 15 (e.g., about 8.2), the number y is about 35, 36, 37, 38, 39, 40, 41, 42, 43, 44, 45, 46, 47, 48, 49, 49.5, 49.6, 49.7, 49.8, 49.9, 50, 50.1, 50.2, 50.3, 50.4, 50.5, 50.6, 50.7, 50.8, 50.9, 51, 52, 53, 54, 55, 56, 57, 58, 59, 60, 61, 62, 63, 64, 65, 66, 67, 68, 69, or 70 (e.g., about 50 or about 50.3), and the number z is 1, 2, 3, 4, 5, 6, 7, 7.5, 7.6, 7.7, 7.8, 7.9, 8, 8.1, 8.2, 8.3, 8.4, 8.5, 8.6, 8.7, 8.8, 8.9, 9, 10, 11, 12, 13, 14, or 15 (e.g., about 8.2). In some embodiments, the number x is about 1, 2, 2.5, 2.6, 2.7, 2.8. 2.9, 3, 3.1, 3.2, 3.3, 3.4, 3.5, 3.6, 3.7, 3.8, 3.9, 4, 5, 6, 7, 8, 9, or (e.g., about 3 or about 3.1), the number y is about 20, 21, 22, 23, 24, 25, 26, 27, 28, 29, 30, 31, 32, 33, 34, 35, 36, 37, 38, 39, 40, 41, 42, 42.1, 42.2, 42.3, 42.4, 42.5, 42.6, 42.7, 42.8, 42.9, 43, 43.1, 43.2, 43.3, 43.4, 43.5, 44, 45, 46, 47, 48, 49, 50, 51, 52, 53, 54, 55, 56, 57, 58, 59, or 60 (e.g., about 42.6 or about 43), and the number z is about 1, 2, 2.5, 2.6, 2.7, 2.8. 2.9, 3, 3.1, 3.2, 3.3, 3.4, 3.5, 3.6, 3.7, 3.8, 3.9, 4, 5, 6, 7, 8, 9, or 10 (e.g., about 3 or about 3.1). In some embodiments, the numbers (e.g., x, y, and z) described herein are average numbers.

In some embodiments, the weight percentage of PEO (PEO % (w/w)) is less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of formula I. In some embodiments, the weight percentage of PPO (PPO % (w/w)) is greater than 80%, greater than 81%, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, or greater than 99%. In some embodiments, the weight percentage of PEO is between about 1% to about 20%, about 1% to about 15%, about 1% to about 10%, about 1% to about 5%, about 5% to about 20%, about 5% to about 15%, about 5% to about 10%, about 10% to about 20%, about 10% to about 15%, or about 15% to about 20%.

In some embodiments, the average molecular weight of formula I is about 1000 Daltons to about 30000 Daltons. In some embodiments, the average molecular weight of formula I is about 1000 Daltons to about 20000 Daltons, about 1000 Daltons to about 10000 Daltons, about 1000 Daltons to about 9000 Daltons, about 1000 Daltons to about 8000 Daltons, about 1000 Daltons to about 7000 Daltons, about 1000 Daltons to about 6000 Daltons, about 1000 Daltons to about 5000 Daltons, about 1000 Daltons to about 4000 Daltons, about 1000 Daltons to about 3000 Daltons, about 1000 Daltons to about 2000 Daltons, about 2000 Daltons to about 10000 Daltons, about 2000 Daltons to about 9000 Daltons, about 2000 Daltons to about 8000 Daltons, about 2000 Daltons to about 7000 Daltons, about 2000 Daltons to about 6000 Daltons, about 2000 Daltons to about 5000 Daltons, about 2000 Daltons to about 4000 Daltons, about 2000 Daltons to about 3000 Daltons, about 3000 Daltons to about 10000 Daltons, about 3000 Daltons to about 9000 Daltons, about 3000 Daltons to about 8000 Daltons, about 3000 Daltons to about 7000 Daltons, about 3000 Daltons to about 6000 Daltons, about 3000 Daltons to about 5000 Daltons, about 3000 Daltons to about 4000 Daltons, about 4000 Daltons to about 10000 Daltons, about 4000 Daltons to about 9000 Daltons, about 4000 Daltons to about 8000 Daltons, about 4000 Daltons to about 7000 Daltons, about 4000 Daltons to about 6000 Daltons, about 4000 Daltons to about 5000 Daltons, about 5000 Daltons to about 10000 Daltons, about 5000 Daltons to about 9000 Daltons, about 5000 Daltons to about 8000 Daltons, about 5000 Daltons to about 7000 Daltons, about 5000 Daltons to about 6000 Daltons, about 6000 Daltons to about 10000 Daltons, about 6000 Daltons to about 9000 Daltons, about 6000 Daltons to about 8000 Daltons, about 6000 Daltons to about 7000 Daltons, about 7000 Daltons to about 10000 Daltons, about 7000 Daltons to about 9000 Daltons, about 7000 Daltons to about 8000 Daltons, about 8000 Daltons to about 10000 Daltons, about 8000 Daltons to about 9000 Daltons, or about 9000 Daltons to about 10000 Daltons. In some embodiments, the average molecular weight of formula I is about 2000 Daltons, about 2500 Daltons, about 2600 Daltons, about 2700 Daltons, about 2750 Daltons, about 2800 Daltons, about 2900 Daltons, about 3000 Daltons, about 3100 Daltons, about 3200 Daltons, about 3300 Daltons, about 3400 Daltons, about 3500 Daltons, about 3600 Daltons, about 3650 Daltons, about 3700 Daltons, about 3750 Daltons, about 3800 Daltons, about 3900 Daltons, about 4000 Daltons, about 4100 Daltons, about 4200 Daltons, about 4300 Daltons, about 4400 Daltons, about 4500 Daltons, about 4600 Daltons, about 4700 Daltons, about 4800 Daltons, about 4900 Daltons, or about 5000 Daltons.

In some embodiments, when the number x and number z are identical, formula I can also be depicted as:

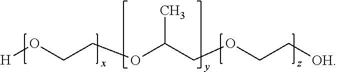

In some embodiments, the above formula is also depicted as PEOₓ-PPO_{y}-PEOₓ, [PEO]ₓ—[PPO]_{y}—[PEO]ₓ, or H[OCH₂CH₂]ₓ[OCH(CH₃)CH₂]_{y}[OCH₂CH₂]ₓOH.

### Formula I (Triblock Co-Polymer)

In some embodiments, the triblock co-polymer of formula I can be inverted, i.e., the PEO block located in the center of the tri-blocks, and two PPO blocks located at the two sides of the PEO block. In some embodiments, the triblock co-polymer is derived from a compound of formula II with a general structure shown below:

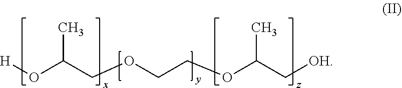

In some embodiments, formula II is also depicted as PPOₓ-PEO_{y}-PPO_{z}, [PPO]ₓ—[PEO]_{y}—[PPO]_{z}, PPO-PEO-PPO, poly(propylene oxide)-poly(ethylene oxide)-poly(propylene oxide), PPG-PEG-PPG, H[OCH(CH₃)CH₂]ₓ[OCH₂CH₂]_{y}[OCH(CH₃)CH₂]:OH. In some embodiments, x, y, and z in formula I or its equivalent formula thereof are numbers. For example, x is a number selected from 10-500, y is a number selected from 1-100, and z is a number selected from 10-500. In some embodiments, the numbers x and z are identical. In some embodiments, the number x and/or number z are at least 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold higher than number y.

In some embodiments, the triblock co-polymer is derived from a compound of formula II′ with a general structure shown below:

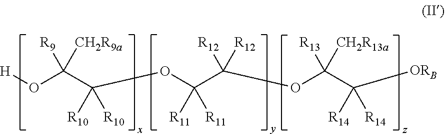

or a salt thereof,
wherein:
each R₉, R₉ₐ, R₁₀, R₁₁, R₁₂, R₁₃, R₁₃ₐ, and R₁₄ is, independently, selected from the group consisting of hydrogen (H), C₁₋₆ alkyl, C₂₋₆ alkenyl, C₂₋₆ alkynyl, C₁₋₆alkoxy, C₁₋₆ haloalkyl, C₁₋₆ haloalkoxy, C₁₋₆ hydroxyalkyl, halo, cyano (—CN), nitro (—NO₂), hydroxyl (OH), carboxyl (—COOH), carboxyaldehyde (—C(O)H), amido (e.g., —C(O)NH₂, —C(O)NHC₁₋₄ alkyl, or —C(O)N(C₁₋₄ alkyl)₂), and amino (e.g., —NH₂, —NHC₁₋₄ alkyl, and —N(C₁₋₄ alkyl)₂); and
R_{B} is selected from the group consisting of hydrogen (H), C₁₋₆ alkyl, C₂₋₆ alkenyl, C₂₋₆ alkynyl, C₁₋₆ haloalkyl, and C₁₋₆ hydroxyalkyl.

In some embodiments, each R₉, R₉ₐ, R₁₀, R₁₁, R₁₂, R₁₃, R₁₃ₐ, R₁₄, and R_{B} is H.

In some embodiments, each R₉, R₉ₐ, R₁₀, R₁₁, R₁₂, R₁₃, R₁₃ₐ, and R₁₄ is H; and R_{B} is methyl.

In some embodiments, R₉ is H. In some embodiments, R₉ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, R₉ is selected from methyl, ethyl, methoxy, ethoxy, hydroxymethyl, halo, and hydroxyl. In some embodiments, R₉ is methyl. In some embodiments, R₉ is ethyl. In some embodiments, R₉ is methoxy. In some embodiments, R₉ is ethoxy. In some embodiments, R₉ is hydroxymethyl. In some embodiments, R₉ is halo. In some embodiments, R₉ is hydroxyl.

In some embodiments, R₉ₐ is H. In some embodiments, R₉ₐ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, R₉ₐ is selected from methyl, ethyl, methoxy, ethoxy, hydroxymethyl, halo, and hydroxyl. In some embodiments, R₉ₐ is methyl. In some embodiments, R₉ₐ is ethyl. In some embodiments, R₉ₐ is methoxy. In some embodiments, R₉ₐ is ethoxy. In some embodiments, R₉ₐ is hydroxymethyl. In some embodiments, R₉ₐ is halo. In some embodiments, R₉ₐ is hydroxyl.

In some embodiments, each R₁₀ is H. In some embodiments, at least one R₁₀ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, at least one R₁₀ is selected from methyl, ethyl, methoxy, ethoxy, hydroxymethyl, halo, and hydroxyl. In some embodiments, at least one R₁₀ is methyl. In some embodiments, at least one R₁₀ is ethyl. In some embodiments, at least one R₁₀ is methoxy. In some embodiments, at least one R₁₀ is ethoxy. In some embodiments, at least one R₁₀ is hydroxymethyl. In some embodiments, at least one R₁₀ is halo. In some embodiments, at least one R₁₀ is hydroxyl.

In some embodiments, each R₁₁ is H. In some embodiments, at least one R₁₁ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, at least one R₁₁ is selected from methyl, ethyl, methoxy, ethoxy, hydroxymethyl, halo, and hydroxyl. In some embodiments, at least one R₁₁ is methyl. In some embodiments, at least one R₁₁ is ethyl. In some embodiments, at least one R₁₁ is methoxy. In some embodiments, at least one R₁₁ is ethoxy. In some embodiments, at least one R₁₁ is hydroxymethyl. In some embodiments, at least one R₁₁ is halo. In some embodiments, at least one R₁₁ is hydroxyl.

In some embodiments, each R₁₂ is H. In some embodiments, at least one R₁₂ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, at least one R₁₂ is selected from methyl, ethyl, methoxy, ethoxy, hydroxymethyl, halo, and hydroxyl. In some embodiments, at least one R₁₂ is methyl. In some embodiments, at least one R₁₂ is ethyl. In some embodiments, at least one R₁₂ is methoxy. In some embodiments, at least one R₁₂ is ethoxy. In some embodiments, at least one R₁₂ is hydroxymethyl. In some embodiments, at least one R₁₂ is halo. In some embodiments, at least one R₁₂ is hydroxyl.

In some embodiments, R₁₃ is H. In some embodiments, R₁₃ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, R₁₃ is selected from methyl, ethyl, methoxy, ethoxy, hydroxymethyl, halo, and hydroxyl. In some embodiments, R₁₃ is methyl. In some embodiments, R₁₃ is ethyl. In some embodiments, R₁₃ is methoxy. In some embodiments, R₁₃ is ethoxy. In some embodiments, R₁₃ is hydroxymethyl. In some embodiments, R₁₃ is halo. In some embodiments, R₁₃ is hydroxyl.

In some embodiments, R₁₃ₐ is H. In some embodiments, R₁₃ₐ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, R₁₃ₐ is selected from methyl, ethyl, methoxy, ethoxy, hydroxymethyl, halo, and hydroxyl. In some embodiments, R₁₃ₐ is methyl. In some embodiments, R₁₃ₐ is ethyl. In some embodiments, R₁₃ₐ is methoxy. In some embodiments, R₁₃ₐ is hydroxymethyl. In some embodiments, R₁₃ₐ is ethoxy. In some embodiments, R₁₃ₐ is halo. In some embodiments, R₁₃ₐ is hydroxyl.

In some embodiments, each R₁₄ is H. In some embodiments, at least one R₁₄ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, at least one R₁₄ is selected from methyl, ethyl, methoxy, ethoxy, hydroxymethyl, halo, and hydroxyl. In some embodiments, at least one R₁₄ is methyl. In some embodiments, at least one R₁₄ is ethyl. In some embodiments, at least one R₁₄ is methoxy. In some embodiments, at least one R₁₄ is ethoxy. In some embodiments, at least one R₁₄ is hydroxymethyl. In some embodiments, at least one R₁₄ is halo. In some embodiments, at least one R₁₄ is hydroxyl.

In some embodiments, Ra is H. In some embodiments, Ra is selected from C₁₋₆ alkyl, C₂₋₆ alkenyl, C₂₋₆ alkynyl, C₁₋₆ haloalkyl, and C₁₋₆ hydroxyalkyl. In some embodiments, Ra is selected from methyl and ethyl. In some embodiments, Ra is methyl. In some embodiments, R_{B} is ethyl. In some embodiments, R_{B} is hydroxymethyl.

In some embodiments, the triblock co-polymer is derived from a compound of formula II″ with a general structure shown below:

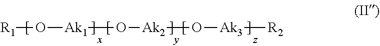

or a salt thereof,
wherein:
R₁ is hydrogen (H), optionally substituted alkyl (e.g., C₁₋₆, C₁₋₄, C₁₋₃, or C₁₋₂ alkyl), or optionally substituted hydroxyalkyl (e.g., C₁₋₆, C₁₋₄, C₁₋₃, or C₁₋₂ hydroxyalkyl);
R₂ is hydroxyl (—OH), optionally substituted alkoxy (e.g., C₁₋₆, C₁₋₄, C₁₋₃, or C₁₋₂ alkoxy), or optionally substituted hydroxyalkyl (e.g., C₁₋₆, C₁₋₄, C₁₋₃, or C₁₋₂ hydroxyalkyl);
each of Ak₁, Ak₂, and Ak₃ is, independently, an optionally substituted alkylene (e.g., optionally substituted C₁₋₆, C₁₋₄, C₁₋₃, or C₁₋₂ alkylene);
each of Ak₁, Ak₂, and Ak₃ is not identical; and
x, y, and z can be any described herein (e.g., as described herein for formula II).

In some embodiments, R₁, R₂, Ak₁, Ak₂, and Ak₃ can be any described herein (e.g., as for formula I″).

In some embodiments, at least one of Ak₁, Ak₂, and Ak₃ is, independently, an unbranched alkylene (e.g., an unbranched C₁₋₆, C₁₋₄, C₁₋₃, or C₁₋₂ alkylene) that may be optionally substituted. In some embodiments, Ak₂ is an unbranched alkylene.

In some embodiments, at least one of Ak₁, Ak₂, and Ak₃ is a branched alkylene (e.g., a branched C₂₋₆, C₂₋₄, C₃₋₆, or C₃₋₄ alkylene) that may be optionally substituted. In some embodiments, each of Ak₁ and Ak₃ is, independently, a branched alkylene.

In some embodiments, the number x is about 10-500, 10-450, 10-400, 10-350, 10-300, 10-250, 10-200, 10-150, 10-100, 10-90, 10-80, 10-70, 10-60, 10-50, 10-40, 10-30, 10-20, 20-500, 20-450, 20-400, 20-350, 20-300, 20-250, 20-200, 20-150, 20-100, 20-90, 20-80, 20-70, 20-60, 20-50, 20-40, 20-30, 30-500, 30-450, 30-400, 30-350, 30-300, 30-250, 30-200, 30-150, 30-100, 30-90, 30-80, 30-70, 30-60, 30-50, 30-40, 40-500, 40-450, 40-400, 40-350, 40-300, 40-250, 40-200, 40-150, 40-100, 40-90, 40-80, 40-70, 40-60, 40-50, 50-500, 50-450, 50-400, 50-350, 50-300, 50-250, 50-200, 50-150, 50-100, 50-90, 50-80, 50-70, 50-60, 60-500, 60-450, 60-400, 60-350, 60-300, 60-250, 60-200, 60-150, 60-100, 60-90, 60-80, 60-70, 70-500, 70-450, 70-400, 70-350, 70-300, 70-250, 70-200, 70-150, 70-100, 70-90, 70-80, 80-500, 80-450, 80-400, 80-350, 80-300, 80-250, 80-200, 80-150, 80-100, 80-90, 90-500, 90-450, 90-400, 90-350, 90-300, 90-250, 90-200, 90-150, 90-100, 100-500, 100-450, 100-400, 100-350, 100-300, 100-250, 100-200, 100-150, 150-500, 150-450, 150-400, 150-350, 150-300, 150-250, 150-200, 200-500, 200-450, 200-400, 200-350, 200-300, 200-250, 250-500, 250-450, 250-400, 250-350, 250-300, 300-500, 300-450, 300-400, 300-350, 350-500, 350-450, 350-400, 400-500, 400-450, or 450-500; the number y is about 1-100, 1-90, 1-80, 1-70, 1-60, 1-50, 1-40, 1-30, 1-20, 1-10, 1-5, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100; and the number z is about 10-500, 10-450, 10-400, 10-350, 10-300, 10-250, 10-200, 10-150, 10-100, 10-90, 10-80, 10-70, 10-60, 10-50, 10-40, 10-30, 10-20, 20-500, 20-450, 20-400, 20-350, 20-300, 20-250, 20-200, 20-150, 20-100, 20-90, 20-80, 20-70, 20-60, 20-50, 20-40, 20-30, 30-500, 30-450, 30-400, 30-350, 30-300, 30-250, 30-200, 30-150, 30-100, 30-90, 30-80, 30-70, 30-60, 30-50, 30-40, 40-500, 40-450, 40-400, 40-350, 40-300, 40-250, 40-200, 40-150, 40-100, 40-90, 40-80, 40-70, 40-60, 40-50, 50-500, 50-450, 50-400, 50-350, 50-300, 50-250, 50-200, 50-150, 50-100, 50-90, 50-80, 50-70, 50-60, 60-500, 60-450, 60-400, 60-350, 60-300, 60-250, 60-200, 60-150, 60-100, 60-90, 60-80, 60-70, 70-500, 70-450, 70-400, 70-350, 70-300, 70-250, 70-200, 70-150, 70-100, 70-90, 70-80, 80-500, 80-450, 80-400, 80-350, 80-300, 80-250, 80-200, 80-150, 80-100, 80-90, 90-500, 90-450, 90-400, 90-350, 90-300, 90-250, 90-200, 90-150, 90-100, 100-500, 100-450, 100-400, 100-350, 100-300, 100-250, 100-200, 100-150, 150-500, 150-450, 150-400, 150-350, 150-300, 150-250, 150-200, 200-500, 200-450, 200-400, 200-350, 200-300, 200-250, 250-500, 250-450, 250-400, 250-350, 250-300, 300-500, 300-450, 300-400, 300-350, 350-500, 350-450, 350-400, 400-500, 400-450, or 450-500. In some embodiments, the number x is selected from 10-50, the number y is selected from 1-30, and the number z is selected from 10-50.

In some embodiments, the number x is about 10, 11, 12, 13, 14, 15, 16, 17, 18, 19, 20, 21, 22, 23, 24, 24.5, 25, 25.1, 25.2, 25.3, 25.4, 25.5, 25.6, 25.7, 25.8, 25.9, 26, 26.1, 26.2, 26.3, 26.4, 26.5, 27, 28, 29, or 30 (e.g., about 25.6), the number y is about 1, 2, 3, 4, 5, 6, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 9, 10, 11, 12, 13, 14, or 15 (e.g., about 7.5), and the number z is about 10, 11, 12, 13, 14, 15, 16, 17, 18, 19, 20, 21, 22, 23, 24, 24.5, 25, 25.1, 25.2, 25.3, 25.4, 25.5, 25.6, 25.7, 25.8, 25.9, 26, 26.1, 26.2, 26.3, 26.4, 26.5, 27, 28, 29, or 30 (e.g., about 25.6). In some embodiments, the number x is about 10, 11, 12, 13, 13.5, 13.6, 13.7, 13.8. 13.9, 14, 14.1, 14.2, 14.3, 14.4, 14.5, 15, 16, 17, 18, 19, 20, 21, 22, 23, 24, or 25 (e.g., about 14), the number y is about 20, 21, 22, 23, 24, 24.1, 24.2, 24.3, 24.4, 24.5, 24.6, 24.7, 24.8, 24.9, 25, 26, 27, 28, 29, 30, 31, 32, 33, 34, or 35 (e.g., about 24.5), and the number z is about 10, 11, 12, 13, 13.5, 13.6, 13.7, 13.8. 13.9, 14, 14.1, 14.2, 14.3, 14.4, 14.5, 15, 16, 17, 18, 19, 20, 21, 22, 23, 24, or 25 (e.g., about 14). In some embodiments, the numbers (e.g., x, y, and z) described herein are average numbers.

In some embodiments, the weight percentage of PEO (PEO % (w/w)) is less than 40%, less than 35%, less than 30%, less than 25%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of formula I. In some embodiments, the weight percentage of PPO (PPO % (w/w)) is greater than 60%, greater than 65%, greater than 70%, greater than 75%, greater than 80%, greater than 81%, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, or greater than 99%. In some embodiments, the weight percentage of PEO is between about 1% to about 20%, about 1% to about 15%, about 1% to about 10%, about 1% to about 5%, about 5% to about 20%, about 5% to about 15%, about 5% to about 10%, about 10% to about 20%, about 10% to about 15%, or about 15% to about 20%.

In some embodiments, the average molecular weight of formula II is about 1000 Daltons to about 30000 Daltons. In some embodiments, the average molecular weight of formula II is about 1000 Daltons to about 20000 Daltons, about 1000 Daltons to about 10000 Daltons, about 1000 Daltons to about 9000 Daltons, about 1000 Daltons to about 8000 Daltons, about 1000 Daltons to about 7000 Daltons, about 1000 Daltons to about 6000 Daltons, about 1000 Daltons to about 5000 Daltons, about 1000 Daltons to about 4000 Daltons, about 1000 Daltons to about 3000 Daltons, about 1000 Daltons to about 2000 Daltons, about 2000 Daltons to about 10000 Daltons, about 2000 Daltons to about 9000 Daltons, about 2000 Daltons to about 8000 Daltons, about 2000 Daltons to about 7000 Daltons, about 2000 Daltons to about 6000 Daltons, about 2000 Daltons to about 5000 Daltons, about 2000 Daltons to about 4000 Daltons, about 2000 Daltons to about 3000 Daltons, about 3000 Daltons to about 10000 Daltons, about 3000 Daltons to about 9000 Daltons, about 3000 Daltons to about 8000 Daltons, about 3000 Daltons to about 7000 Daltons, about 3000 Daltons to about 6000 Daltons, about 3000 Daltons to about 5000 Daltons, about 3000 Daltons to about 4000 Daltons, about 4000 Daltons to about 10000 Daltons, about 4000 Daltons to about 9000 Daltons, about 4000 Daltons to about 8000 Daltons, about 4000 Daltons to about 7000 Daltons, about 4000 Daltons to about 6000 Daltons, about 4000 Daltons to about 5000 Daltons, about 5000 Daltons to about 10000 Daltons, about 5000 Daltons to about 9000 Daltons, about 5000 Daltons to about 8000 Daltons, about 5000 Daltons to about 7000 Daltons, about 5000 Daltons to about 6000 Daltons, about 6000 Daltons to about 10000 Daltons, about 6000 Daltons to about 9000 Daltons, about 6000 Daltons to about 8000 Daltons, about 6000 Daltons to about 7000 Daltons, about 7000 Daltons to about 10000 Daltons, about 7000 Daltons to about 9000 Daltons, about 7000 Daltons to about 8000 Daltons, about 8000 Daltons to about 10000 Daltons, about 8000 Daltons to about 9000 Daltons, or about 9000 Daltons to about 10000 Daltons. In some embodiments, the average molecular weight of formula II is about 2000 Daltons, about 2100 Daltons, about 2200 Daltons, about 2300 Daltons, about 2400 Daltons, about 2500 Daltons, about 2600 Daltons, about 2700 Daltons, about 2800 Daltons, about 2900 Daltons, about 3000 Daltons, about 3100 Daltons, about 3200 Daltons, about 3300 Daltons, about 3400 Daltons, about 3500 Daltons, about 3600 Daltons, about 3700 Daltons, about 3750 Daltons, about 3800 Daltons, about 3900 Daltons, about 4000 Daltons, about 4100 Daltons, about 4200 Daltons, about 4300 Daltons, about 4400 Daltons, about 4500 Daltons, about 4600 Daltons, about 4700 Daltons, about 4800 Daltons, about 4900 Daltons, or about 5000 Daltons.

In some embodiments, when the number x and number z are identical, formula II can also be depicted as:

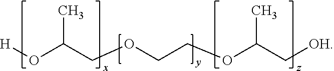

In some embodiments, the above formula is also depicted as PPOₓ-PEO_{y}-PPO_{z}, [PPO]ₓ—[PEO]_{y}—[PPO]ₓ, H[OCH(CH₃)CH₂]ₓ[OCH₂CH₂]_{y}[OCH(CH₃)CH₂]ₓOH.

### Formula III (Diblock Co-Polymer)

In some embodiments, the polymer component described herein includes a diblock co-polymer.

In some embodiments, the triblock co-polymer is derived from a compound of formula I with a general structure shown below:

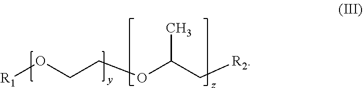

In some embodiments, R₁ is H or CH₃, and R₂ is OH or OCH₃.

In some embodiments, formula III is also depicted as PEO_{y}-PPO_{z}, PEO]_{y}—[PPO]_{z}, PEO-PPO, poly(ethylene oxide)-poly(propylene oxide), PEG-PPG, or R₁[OCH₂CH₂]_{y}[OCH(CH₃)CH₂]_{z}R2. In some embodiments, y and z in formula III or its equivalent formula thereof are numbers. For example, y is a number selected from 1-100, and z is a number selected from 5-500. In some embodiments, the numbers x and z are identical.

In some embodiments, the numbers x and z are not identical. In some embodiments, the number z are at least 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold higher than number y.

In some embodiments, the triblock co-polymer is derived from a compound of formula III′ with a general structure shown below:

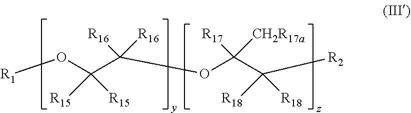

or a salt thereof,
wherein:
R₁ is hydrogen (H) or alkyl (e.g., C₁₋₆ alkyl, such as CH₃);
R₂ is hydroxyl (—OH) or alkoxy (e.g., C₁₋₆ alkoxy, such as —OCH₃); and
each R₁₅, R₁₆, R₁₇, R₁₇ₐ, and R₁₈ is, independently, selected from the group consisting of hydrogen (H), C₁₋₆ alkyl, C₂₋₆ alkenyl, C₂₋₆ alkynyl, C₁₋₆ alkoxy, C₁₋₆ haloalkyl, C₁₋₆ haloalkoxy, C₁₋₆ hydroxyalkyl, halo, cyano (—CN), nitro (—NO₂), hydroxyl (—OH), carboxyl (—COOH), carboxyaldehyde (—C(O)H), amido (e.g., —C(O)NH₂, —C(O)NHC₁₋₄ alkyl, or —C(O)N(C₁₋₄ alkyl)₂), and amino (e.g., —NH₂, —NHC₁₋₄ alkyl, and —N(C₁₋₄ alkyl)₂).

In some embodiments, R₁ is H. In some embodiments, R₁ is CH₃.

In some embodiments, R₂ is OH. In some embodiments, R₂ is OCH₃.

In some embodiments, R₁ is H, and R₂ is OH. In some embodiments, R₁ is H, and R₂ is OCH₃. In some embodiments, R₁ is CH₃, and R₂ is OH. In some embodiments, R₁ is CH₃, and R₂ is OCH₃.

In some embodiments, each R₁₅, R₁₆, R₁₇, R₁₇ₐ, and R₁₈ is H.

In some embodiments, each R₁₅ is H. In some embodiments, at least one R₁₅ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, at least one R₁₅ is selected from methyl, ethyl, methoxy, ethoxy, hydroxymethyl, halo, and hydroxyl. In some embodiments, at least one R₁₅ is methyl. In some embodiments, at least one R₁₅ is ethyl. In some embodiments, at least one R₁₅ is methoxy. In some embodiments, at least one R₁₅ is ethoxy. In some embodiments, at least one R₁₅ is hydroxymethyl. In some embodiments, at least one R₁₅ is halo. In some embodiments, at least one R₁₅ is hydroxyl.

In some embodiments, each R₁₆ is H. In some embodiments, at least one R₁₆ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, at least one R₁₆ is selected from methyl, ethyl, methoxy, ethoxy, hydroxymethyl, halo, and hydroxyl. In some embodiments, at least one R₁₆ is methyl. In some embodiments, at least one R₁₆ is ethyl. In some embodiments, at least one R₁₆ is methoxy. In some embodiments, at least one R₁₆ is ethoxy. In some embodiments, at least one R₁₆ is hydroxymethyl. In some embodiments, at least one R₁₆ is halo. In some embodiments, at least one R₁₆ is hydroxyl.

In some embodiments, R₁₇ is H. In some embodiments, R₁₇ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, R₁₇ is selected from methyl, ethyl, methoxy, ethoxy, hydroxymethyl, halo, and hydroxyl. In some embodiments, R₁₇ is methyl. In some embodiments, R₁₇ is ethyl. In some embodiments, R₁₇ is methoxy. In some embodiments, R₁₇ is ethoxy. In some embodiments, R₁₇ is hydroxymethyl. In some embodiments, R₁₇ is halo. In some embodiments, R₁₇ is hydroxyl.

In some embodiments, Ria is H. In some embodiments, R₁₇ₐ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, R₁₇ₐ is selected from methyl, ethyl, methoxy, ethoxy, hydroxymethyl, halo, and hydroxyl. In some embodiments, Ria is methyl. In some embodiments, R₁₇ₐ is ethyl. In some embodiments, R₁₇ₐ is methoxy. In some embodiments, R₁₇ₐ is ethoxy. In some embodiments, R₁₇ₐ is hydroxymethyl. In some embodiments, R₁₇ₐ is halo. In some embodiments, R₁₇ₐ is hydroxyl.

In some embodiments, each R₁₈ is H. In some embodiments, at least one R₁₈ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, at least one R₁₈ is selected from methyl, ethyl, methoxy, ethoxy, hydroxymethyl, halo, and hydroxyl. In some embodiments, at least one R₁₈ is methyl. In some embodiments, at least one R₁₈ is ethyl. In some embodiments, at least one R₁₈ is methoxy. In some embodiments, at least one R₁₈ is ethoxy. In some embodiments, at least one R₁₈ is hydroxymethyl. In some embodiments, at least one R₁₈ is halo. In some embodiments, at least one R₁₈ is hydroxyl.

In some embodiments, the triblock co-polymer is derived from a compound of formula III″ with a general structure shown below:

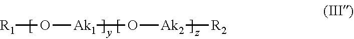

or a salt thereof,
wherein:
R₁ is hydrogen (H), optionally substituted alkyl (e.g., C₁₋₆, C₁₋₄, C₁₋₃, or C₁₋₂ alkyl), or optionally substituted hydroxyalkyl (e.g., C₁₋₆, C₁₋₄, C₁₋₃, or C₁₋₂ hydroxyalkyl);
R₂ is hydroxyl (—OH), optionally substituted alkoxy (e.g., C₁₋₆, C₁₋₄, C₁₋₃, or C₁₋₂ alkoxy), or optionally substituted hydroxyalkyl (e.g., C₁₋₆, C₁₋₄, C₁₋₃, or C₁₋₂ hydroxyalkyl);
each of Ak₁ and Ak₂ is, independently, an optionally substituted alkylene (e.g., optionally substituted C₁₋₆, C₁₋₄, C₁₋₃, or C₁₋₂ alkylene);
each of Ak₁ and Ak₂ is not identical; and
y and z is can be any described herein (e.g., as described herein for formula III).

In some embodiments, R₁ is hydrogen or C₁₋₆ alkyl. In some embodiments, R₂ is hydroxyl or C₁₋₆ alkoxy. In some embodiments, R₁ is hydrogen, and R₂ is hydroxyl. In some embodiments, R₁ is hydrogen, and R₂ is C₁₋₆ alkoxy. In some embodiments, R₁ is C₁₋₆ alkyl, and R₂ is hydroxyl. In some embodiments, R₁ is C₁₋₆ alkyl, and R₂ is C₁₋₆ alkoxy.

In some embodiments, the optionally substituted alkylene is substituted with one or more substituents. Non-limiting examples of substituents include one or more of the following: halo, haloalkyl, alkoxy, hydroxyalkyl, amino, amido, cyano, nitro, hydroxyl, carboxyl, oxo, carboxyaldehyde, or a combination thereof.

In some embodiments, at least one of Ak₁ and Ak₂ is, independently, an optionally substituted C₁₋₆, C₁₋₆, C₁₋₃, or C₁₋₂ alkylene. In some embodiments, each of Ak₁ and Ak₂ is, independently, an optionally substituted C₁₋₆, C₁₋₄, C₁₋₃, or C₁₋₂ alkylene.

In some embodiments, at least one of Ak₁ and Ak₂ is, independently, an unsubstituted C₁₋₆, C₁₋₄, C₁₋₃, or C₁₋₂ alkylene. In some embodiments, each of Ak₁ and Ak₂ is, independently, an unsubstituted C₁₋₆, C₁₋₄, C₁₋₃, or C₁₋₂ alkylene.

In some embodiments, at least one of Ak₁ and Ak₂ is, independently, an unbranched alkylene (e.g., an unbranched C₁₋₆, C₁₋₆, C₁₋₃, or C₁₋₂ alkylene) that may be optionally substituted. In some embodiments, Ak₁ is an unbranched alkylene.

In some embodiments, at least one of Ak₁ and Ak₂ is a branched alkylene (e.g., a branched C₂₋₆, C₂₋₄, C₃₋₆, or C₃₋₄ alkylene) that may be optionally substituted. In some embodiments, Ak₂ is a branched alkylene.

In some embodiments, the number y is about 1, 2, 3, 4, 5, 6, 7, 8, 9, 10, 15, 20, 25, 30, 35, 40, 45, 50, 55, 60, 65, 70, 75, 80, 85, 90, 95, or 100, and the number z is about 5, 10, 20, 30, 40, 50, 60, 70, 80, 90, 100, 150, 200, 250, 300, 350, 400, 450 or 500. In some embodiments, the number y is about 1-100, 1-90, 1-80, 1-70, 1-60, 1-50, 1-40, 1-30, 1-20, 1-10, 1-5, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100; and the number z is about 1-500, 1-450, 1-400, 1-350, 1-300, 1-250, 1-200, 1-150, 1-100, 1-90, 1-80, 1-70, 1-60, 1-50, 1-40, 1-30, 1-20, 1-10, 1-5, 10-500, 10-450, 10-400, 10-350, 10-300, 10-250, 10-200, 10-150, 10-100, 10-90, 10-80, 10-70, 10-60, 10-50, 10-40, 10-30, 10-20, 20-500, 20-450, 20-400, 20-350, 20-300, 20-250, 20-200, 20-150, 20-100, 20-90, 20-80, 20-70, 20-60, 20-50, 20-40, 20-30, 30-500, 30-450, 30-400, 30-350, 30-300, 30-250, 30-200, 30-150, 30-100, 30-90, 30-80, 30-70, 30-60, 30-50, 30-40, 40-500, 40-450, 40-400, 40-350, 40-300, 40-250, 40-200, 40-150, 40-100, 40-90, 40-80, 40-70, 40-60, 40-50, 50-500, 50-450, 50-400, 50-350, 50-300, 50-250, 50-200, 50-150, 50-100, 50-90, 50-80, 50-70, 50-60, 60-500, 60-450, 60-400, 60-350, 60-300, 60-250, 60-200, 60-150, 60-100, 60-90, 60-80, 60-70, 70-500, 70-450, 70-400, 70-350, 70-300, 70-250, 70-200, 70-150, 70-100, 70-90, 70-80, 80-500, 80-450, 80-400, 80-350, 80-300, 80-250, 80-200, 80-150, 80-100, 80-90, 90-500, 90-450, 90-400, 90-350, 90-300, 90-250, 90-200, 90-150, 90-100, 100-500, 100-450, 100-400, 100-350, 100-300, 100-250, 100-200, 100-150, 150-500, 150-450, 150-400, 150-350, 150-300, 150-250, 150-200, 200-500, 200-450, 200-400, 200-350, 200-300, 200-250, 250-500, 250-450, 250-400, 250-350, 250-300, 300-500, 300-450, 300-400, 300-350, 350-500, 350-450, 350-400, 400-500, 400-450, or 450-500.

In some embodiments, the weight percentage of PEO (PEO % (w/w)) is less than 40%, less than 35%, less than 30%, less than 25%, less than 20%, less than 19%, less than 18%%, less than 17%, less than 16%, less than 15%, less than 14%, less than 13%, less than 12%, less than 11%, less than 10%, less than 9%, less than 8%, less than 7%, less than 60%, less than 5%, less than 4%, less than 3%, less than 2%, or less than 1% of formula I. In some embodiments, the weight percentage of PPO (PPO % (w/w)) is greater than 60%, greater than 65%, greater than 70%, greater than 75%, greater than 80%, greater than 81a %, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, or greater than 99%. In some embodiments, the weight percentage of PEO is between about 1% to about 20%, about 1% to about 15%%, about 1% to about 10%, about 1% to about 5%, about 5% to about 20%, about 5% to about 15%, about 5% to about 10%, about 10% to about 20%, about 10% to about 15%, or about 15% to about 20%.

In some embodiments, the average molecular weight of formula II is about 1000 Daltons to about 30000 Daltons. In some embodiments, the average molecular weight of formula II is about 1000 Daltons to about 20000 Daltons, about 1000 Daltons to about 10000 Daltons, about 1000 Daltons to about 9000 Daltons, about 1000 Daltons to about 8000 Daltons, about 1000 Daltons to about 7000 Daltons, about 1000 Daltons to about 6000 Daltons, about 1000 Daltons to about 5000 Daltons, about 1000 Daltons to about 4000 Daltons, about 1000 Daltons to about 3000 Daltons, about 1000 Daltons to about 2000 Daltons, about 2000 Daltons to about 10000 Daltons, about 2000 Daltons to about 9000 Daltons, about 2000 Daltons to about 8000 Daltons, about 2000 Daltons to about 7000 Daltons, about 2000 Daltons to about 6000 Daltons, about 2000 Daltons to about 5000 Daltons, about 2000 Daltons to about 4000 Daltons, about 2000 Daltons to about 3000 Daltons, about 3000 Daltons to about 10000 Daltons, about 3000 Daltons to about 9000 Daltons, about 3000 Daltons to about 8000 Daltons, about 3000 Daltons to about 7000 Daltons, about 3000 Daltons to about 6000 Daltons, about 3000 Daltons to about 5000 Daltons, about 3000 Daltons to about 4000 Daltons, about 4000 Daltons to about 10000 Daltons, about 4000 Daltons to about 9000 Daltons, about 4000 Daltons to about 8000 Daltons, about 4000 Daltons to about 7000 Daltons, about 4000 Daltons to about 6000 Daltons, about 4000 Daltons to about 5000 Daltons, about 5000 Daltons to about 10000 Daltons, about 5000 Daltons to about 9000 Daltons, about 5000 Daltons to about 8000 Daltons, about 5000 Daltons to about 7000 Daltons, about 5000 Daltons to about 6000 Daltons, about 6000 Daltons to about 10000 Daltons, about 6000 Daltons to about 9000 Daltons, about 6000 Daltons to about 8000 Daltons, about 6000 Daltons to about 7000 Daltons, about 7000 Daltons to about 10000 Daltons, about 7000 Daltons to about 9000 Daltons, about 7000 Daltons to about 8000 Daltons, about 8000 Daltons to about 10000 Daltons, about 8000 Daltons to about 9000 Daltons, or about 9000 Daltons to about 10000 Daltons. In some embodiments, the average molecular weight of formula II is about 2000 Daltons, about 2100 Daltons, about 2200 Daltons, about 2300 Daltons, about 2400 Daltons, about 2500 Daltons, about 2600 Daltons, about 2700 Daltons, about 2750 Daltons, about 2800 Daltons, about 2900 Daltons, about 3000 Daltons, about 3100 Daltons, about 3200 Daltons, about 3300 Daltons, about 3400 Daltons, about 3500 Daltons, about 3600 Daltons, about 3700 Daltons, about 3750 Daltons, about 3800 Daltons, about 3900 Daltons, about 4000 Daltons, about 4100 Daltons, about 4200 Daltons, about 4300 Daltons, about 4400 Daltons, about 4500 Daltons, about 4600 Daltons, about 4700 Daltons, about 4800 Daltons, about 4900 Daltons, or about 5000 Daltons.

### Formula IV (Polymer)

In some embodiments, the polymer component described herein includes a polymer that contains a poly (polypropylene oxide), or, poly(ethylene glycol). In some embodiments, the polymer is derived from a compound of formula IV with a general structure shown below:

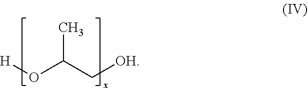

In some embodiments, the formula IV is also depicted as [PPO]ₓ, PPG, PPO, poly(propylene oxide), or H[OCH(CH₃)CH₂]ₓOH.

In some embodiments, the triblock co-polymer is derived from a compound of formula IV′ with a general structure shown below:

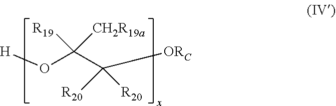

or a salt thereof,
wherein:
each R₁, R₁₉ₐ, and R₂₀ is, independently, selected from the group consisting of hydrogen (H), C₁₋₆ alkyl, C₂₋₆ alkenyl, C₂₋₆ alkynyl, C₁₋₆ alkoxy, C₁₋₆ haloalkyl, C₁₋₆ haloalkoxy, C₁₋₆ hydroxyalkyl, halo, cyano (—CN), nitro (—NO₂), hydroxyl (—OH), carboxyl (—COOH), carboxyaldehyde (—C(O)H), amido (e.g., —C(O)NH₂, —C(O)NHC₁₋₄ alkyl, or —C(O)N(C₁₋₄ alkyl)₂), and amino (e.g., —NH₂, —NHC₁₋₄ alkyl, and —N(C₁₋₄ alkyl)₂); and
R_{C} is selected from the group consisting of hydrogen (H), C₁₋₆ alkyl, C₂₋₆ alkenyl, C₂₋₆ alkynyl, C₁₋₆ haloalkyl, and C₁₋₆ hydroxyalkyl.

In some embodiments, each R₁, R₁₉ₐ, R₂₀, and R_{C} is H.

In some embodiments, each R₁, R₁₉ₐ, and R₂₀ is H; and R_{C} is methyl.

In some embodiments, R₁₉ is H. In some embodiments, R₁₉ is selected from C₁₋₆ alkyl, C₁₋₆ alkoxy, C₁₋₆ haloalkyl, C₁₋₆haloalkoxy, C₁₋₆ hydroxyalkyl, halo, CN, and OH. In some embodiments, R₁₉ is selected from methyl, ethyl, methoxy, ethoxy, hydroxymethyl, halo, and hydroxyl. In some embodiments, R₁₉ is methyl. In some embodiments, R₁₉ is ethyl. In some embodiments, R₁₉ is methoxy. In some embodiments, R₁₉ is ethoxy. In some embodiments, R₁₉ is hydroxymethyl. In some embodiments, R₁₉ is halo. In some embodiments, R₁₉ is hydroxyl.

In some embodiments, R₁₉ₐ is H. In some embodiments, R₁₉ₐ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, R₁₉ₐ is selected from methyl, ethyl, methoxy, ethoxy, hydroxymethyl, halo, and hydroxyl. In some embodiments, R₁₉ₐ is methyl. In some embodiments, R₁₉ₐ is ethyl. In some embodiments, R₁₉ₐ is methoxy. In some embodiments, R₁₉ₐ is ethoxy. In some embodiments, R₁₉ₐ is hydroxymethyl. In some embodiments, R₁₉ₐ is halo. In some embodiments, R₁₉ₐ is hydroxyl.

In some embodiments, each R₂₀ is H. In some embodiments, at least one R₂₀ is selected from C₁₋₆ alkyl, C₁₋₆ alkoxy, C₁₋₆ haloalkyl, C₁₋₆ haloalkoxy, C₁₋₆ hydroxyalkyl, halo, CN, and OH. In some embodiments, at least one R₂₀ is selected from methyl, ethyl, methoxy, ethoxy, hydroxymethyl, halo, and hydroxyl. In some embodiments, at least one R₂₀ is methyl. In some embodiments, at least one R₂₀ is ethyl. In some embodiments, at least one R₂₀ is methoxy. In some embodiments, at least one R₂₀ is ethoxy. In some embodiments, at least one R₂₀ is hydroxymethyl. In some embodiments, at least one R₂₀ is halo. In some embodiments, at least one R₂₀ is hydroxyl.

In some embodiments, R_{C} is H. In some embodiments, R_{C} is selected from C₁₋₆ alkyl, C₂₋₆ alkenyl, C₂₋₆ alkynyl, C₁₋₆ haloalkyl, and C₁₋₆ hydroxyalkyl. In some embodiments, R_{C} is selected from methyl and ethyl. In some embodiments, R_{C} is methyl. In some embodiments, R_{C} is ethyl. In some embodiments, R_{C} is hydroxymethyl.

In some embodiments, the triblock co-polymer is derived from a compound of formula IV″ with a general structure shown below:

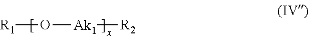

or a salt thereof,
wherein:
R₁ is hydrogen (H), optionally substituted alkyl (e.g., C₁₋₆, C₁₋₄, C₁₋₃, or C₁₋₂ alkyl), or optionally substituted hydroxyalkyl (e.g., C₁₋₆, C₁₋₄, C₁₋₃, or C₁₋₂ hydroxyalkyl);
R₂ is hydroxyl (—OH), optionally substituted alkoxy (e.g., C₁₋₆, C₁₋₄, C₁₋₃, or C₁₋₂ alkoxy), or optionally substituted hydroxyalkyl (e.g., C₁₋₆, C₁₋₄, C₁₋₃, or C₁₋₂ hydroxyalkyl);
Ak₁ is an optionally substituted alkylene (e.g., optionally substituted C₁₋₆, C₁₋₄, C₁₋₃, or C₁₋₂ alkylene); and
x can be any described herein (e.g., as described herein for formula IV).

In some embodiments, R₁ is hydrogen or C₁₋₆ alkyl. In some embodiments, R₂ is hydroxyl or C₁₋₆ alkoxy. In some embodiments, R₁ is hydrogen, and R₂ is hydroxyl. In some embodiments, R₁ is hydrogen, and R₂ is C₁₋₆ alkoxy. In some embodiments, R₁ is C₁₋₆ alkyl, and R₂ is hydroxyl. In some embodiments, R₁ is C₁₋₆ alkyl, and R₂ is C₁₋₆ alkoxy.

In some embodiments, the optionally substituted alkylene is substituted with one or more substituents. Non-limiting examples of substituents include one or more of the following: halo, haloalkyl, alkoxy, hydroxyalkyl, amino, amido, cyano, nitro, hydroxyl, carboxyl, oxo, carboxyaldehyde, or a combination thereof.

In some embodiments, Ak₁ is an optionally substituted C₁₋₆, C₁₋₄, C₁₋₃, or C₁₋₂ alkylene. In some embodiments, Ak₁ is an unsubstituted C₁₋₆, C₁₋₄, C₁₋₃, or C₁₋₂ alkylene.

In some embodiments, Ak₁ is an unbranched alkylene (e.g., an unbranched C₁₋₆, C₁₋₄, C₁₋₃, or C₁₋₂ alkylene) that may be optionally substituted. In some embodiments, Ak₁ is a branched alkylene (e.g., a branched C₂₋₆, C₂₋₄, C₃₋₆, or C₃₋₄ alkylene) that may be optionally substituted.

In some embodiments, x in formula IV or its equivalent formula thereof is a number.

For example, x in a number selected from 1-100, 1-90, 1-80, 1-70, 1-60, 1-50, 1-40, 1-30, 1-20, 1-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100. In some embodiments, the number x is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. In some embodiments, the number x is about 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, or 48 (e.g., about 46.5). In some embodiments, the numbers (e.g., x) described herein are average numbers.

In some embodiments, the average molecular weight of formula IV is about 1000 Daltons to about 30000 Daltons. In some embodiments, the average molecular weight of formula II is about 1000 Daltons to about 20000 Daltons, about 1000 Daltons to about 10000 Daltons, about 1000 Daltons to about 9000 Daltons, about 1000 Daltons to about 8000 Daltons, about 1000 Daltons to about 7000 Daltons, about 1000 Daltons to about 6000 Daltons, about 1000 Daltons to about 5000 Daltons, about 1000 Daltons to about 4000 Daltons, about 1000 Daltons to about 3000 Daltons, about 1000 Daltons to about 2000 Daltons, about 2000 Daltons to about 10000 Daltons, about 2000 Daltons to about 9000 Daltons, about 2000 Daltons to about 8000 Daltons, about 2000 Daltons to about 7000 Daltons, about 2000 Daltons to about 6000 Daltons, about 2000 Daltons to about 5000 Daltons, about 2000 Daltons to about 4000 Daltons, about 2000 Daltons to about 3000 Daltons, about 3000 Daltons to about 10000 Daltons, about 3000 Daltons to about 9000 Daltons, about 3000 Daltons to about 8000 Daltons, about 3000 Daltons to about 7000 Daltons, about 3000 Daltons to about 6000 Daltons, about 3000 Daltons to about 5000 Daltons, about 3000 Daltons to about 4000 Daltons, about 4000 Daltons to about 10000 Daltons, about 4000 Daltons to about 9000 Daltons, about 4000 Daltons to about 8000 Daltons, about 4000 Daltons to about 7000 Daltons, about 4000 Daltons to about 6000 Daltons, about 4000 Daltons to about 5000 Daltons, about 5000 Daltons to about 10000 Daltons, about 5000 Daltons to about 9000 Daltons, about 5000 Daltons to about 8000 Daltons, about 5000 Daltons to about 7000 Daltons, about 5000 Daltons to about 6000 Daltons, about 6000 Daltons to about 10000 Daltons, about 6000 Daltons to about 9000 Daltons, about 6000 Daltons to about 8000 Daltons, about 6000 Daltons to about 7000 Daltons, about 7000 Daltons to about 10000 Daltons, about 7000 Daltons to about 9000 Daltons, about 7000 Daltons to about 8000 Daltons, about 8000 Daltons to about 10000 Daltons, about 8000 Daltons to about 9000 Daltons, or about 9000 Daltons to about 10000 Daltons. In some embodiments, the average molecular weight of formula II is about 2000 Daltons, about 2100 Daltons, about 2200 Daltons, about 2300 Daltons, about 2400 Daltons, about 2500 Daltons, about 2600 Daltons, about 2700 Daltons, about 2750 Daltons, about 2800 Daltons, about 2900 Daltons, about 3000 Daltons, about 3100 Daltons, about 3200 Daltons, about 3300 Daltons, about 3400 Daltons, about 3500 Daltons, about 3600 Daltons, about 3700 Daltons, about 3750 Daltons, about 3800 Daltons, about 3900 Daltons, about 4000 Daltons, about 4100 Daltons, about 4200 Daltons, about 4300 Daltons, about 4400 Daltons, about 4500 Daltons, about 4600 Daltons, about 4700 Daltons, about 4800 Daltons, about 4900 Daltons, or about 5000 Daltons.

### Other Formulas

In some embodiments, the polymer component described herein includes other polymers. e.g., PEO-PPO block polymers described in Alvarez-Lorenzo, C., et al. “PEO-PPO block copolymers for passive micellar targeting and overcoming multidrug resistance in cancer therapy.” Current Drug Targets 12.8 (2011): 1112-1130, for example, poloxamines, which is incorporated herein by reference in its entirety.

In some embodiments, the polymer component described herein includes a polymer that is derived from a compound of formula A with a general structure shown below:

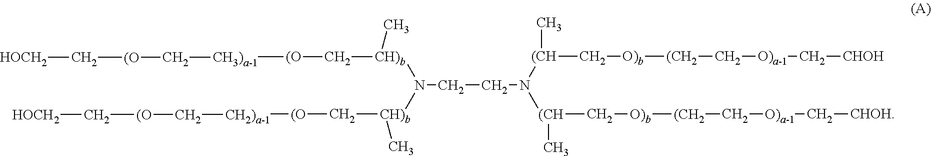

In some embodiments, the polymer component described herein includes a polymer that is derived from a compound of formula B with a general structure shown below:

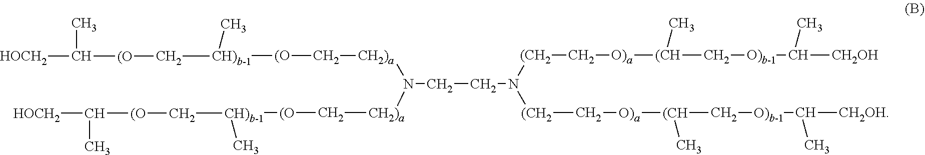

In some embodiments, a in formula A and formula B, or its equivalent formula thereof, is an integral. For example, a in an integral selected from 1-200, 1-190, 1-180, 1-170, 1-160, 1-150, 1-140, 1-130, 1-120, 1-110, 1-100, 1-90, 1-80, 1-70, 1-60, 1-50, 1-40, 1-30, 1-20, 1-10, 2-200, 2-190, 2-180, 2-170, 2-160, 2-150, 2-140, 2-130, 2-120, 2-110, 2-100, 2-90, 2-80, 2-70, 2-60, 2-50, 2-40, 2-30, 2-20, 2-10, 5-200, 5-190, 5-180, 5-170, 5-160, 5-150, 5-140, 5-130, 5-120, 5-110, 5-100, 5-90, 5-80, 5-70, 5-60, 5-50, 5-40, 5-30, 5-20, 5-10, 10-200, 10-190, 10-180, 10-170, 10-160, 10-150, 10-140, 10-130, 10-120, 10-110, 10-100, 10-90, 10-80, 10-70, 10-60, 10-50, 10-40, 10-30, 10-20, 20-200, 20-190, 20-180, 20-170, 20-160, 20-150, 20-140, 20-130, 20-120, 20-110, 20-100, 20-90, 20-80, 20-70, 20-60, 20-50, 20-40, 20-30, 30-200, 30-190, 30-180, 30-170, 30-160, 30-150, 30-140, 30-130, 30-120, 30-110, 30-100, 30-90, 30-80, 30-70, 30-60, 30-50, 30-40, 40-200, 40-190, 40-180, 40-170, 40-160, 40-150, 40-140, 40-130, 40-120, 40-110, 40-100, 40-90, 40-80, 40-70, 40-60, 40-50, 50-200, 50-190, 50-180, 50-170, 50-160, 50-150, 50-140, 50-130, 50-120, 50-110, 50-100, 50-90, 50-80, 50-70, 50-60, 60-200, 60-190, 60-180, 60-170, 60-160, 60-150, 60-140, 60-130, 60-120, 60-110, 60-100, 60-90, 60-80, 60-70, 70-200, 70-190, 70-180, 70-170, 70-160, 70-150, 70-140, 70-130, 70-120, 70-110, 70-100, 70-90, 70-80, 80-200, 80-190, 80-180, 80-170, 80-160, 80-150, 80-140, 80-130, 80-120, 80-110, 80-100, 80-90, 90-200, 90-190, 90-180, 90-170, 90-160, 90-150, 90-140, 90-130, 90-120, 90-110, or 90-100. In some embodiments, the integral a is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200.

In some embodiments, b in formula A and formula B, or its equivalent formula thereof, is an integral. For example, b in an integral selected from 1-300, 1-290, 1-280, 1-270, 1-260, 1-250, 1-240, 1-230, 1-220, 1-210, 1-200, 1-190, 1-180, 1-170, 1-160, 1-150, 1-140, 1-130, 1-120, 1-110, 1-100, 1-90, 1-80, 1-70, 1-60, 1-50, 1-40, 1-30, 1-20, 1-10, 2-300, 2-290, 2-280, 2-270, 2-260, 2-250, 2-240, 2-230, 2-220, 2-210, 2-200, 2-190, 2-180, 2-170, 2-160, 2-150, 2-140, 2-130, 2-120, 2-110, 2-100, 2-90, 2-80, 2-70, 2-60, 2-50, 2-40, 2-30, 2-20, 2-10, 4-300, 4-290, 4-280, 4-270, 4-260, 4-250, 4-240, 4-230, 4-220, 4-210, 4-200, 4-190, 4-180, 4-170, 4-160, 4-150, 4-140, 4-130, 4-120, 4-110, 4-100, 4-90, 4-80, 4-70, 4-60, 4-50, 4-40, 4-30, 4-20, 4-10, 5-300, 5-290, 5-280, 5-270, 5-260, 5-250, 5-240, 5-230, 5-220, 5-210, 5-200, 5-190, 5-180, 5-170, 5-160, 5-150, 5-140, 5-130, 5-120, 5-110, 5-100, 5-90, 5-80, 5-70, 5-60, 5-50, 5-40, 5-30, 5-20, 5-10, 10-300, 10-290, 10-280, 10-270, 10-260, 10-250, 10-240, 10-230, 10-220, 10-210, 10-200, 10-190, 10-180, 10-170, 10-160, 10-150, 10-140, 10-130, 10-120, 10-110, 10-100, 10-90, 10-80, 10-70, 10-60, 10-50, 10-40, 10-30, 10-20, 20-300, 20-290, 20-280, 20-270, 20-260, 20-250, 20-240, 20-230, 20-220, 20-210, 20-200, 20-190, 20-180, 20-170, 20-160, 20-150, 20-140, 20-130, 20-120, 20-110, 20-100, 20-90, 20-80, 20-70, 20-60, 20-50, 20-40, 20-30, 30-300, 30-290, 30-280, 30-270, 30-260, 30-250, 30-240, 30-230, 30-220, 30-210, 30-200, 30-190, 30-180, 30-170, 30-160, 30-150, 30-140, 30-130, 30-120, 30-110, 30-100, 30-90, 30-80, 30-70, 30-60, 30-50, 30-40, 40-300, 40-290, 40-280, 40-270, 40-260, 40-250, 40-240, 40-230, 40-220, 40-210, 40-200, 40-190, 40-180, 40-170, 40-160, 40-150, 40-140, 40-130, 40-120, 40-110, 40-100, 40-90, 40-80, 40-70, 40-60, 40-50, 50-300, 50-290, 50-280, 50-270, 50-260, 50-250, 50-240, 50-230, 50-220, 50-210, 50-200, 50-190, 50-180, 50-170, 50-160, 50-150, 50-140, 50-130, 50-120, 50-110, 50-100, 50-90, 50-80, 50-70, 50-60, 60-300, 60-290, 60-280, 60-270, 60-260, 60-250, 60-240, 60-230, 60-220, 60-210, 60-200, 60-190, 60-180, 60-170, 60-160, 60-150, 60-140, 60-130, 60-120, 60-110, 60-100, 60-90, 60-80, 60-70, 70-300, 70-290, 70-280, 70-270, 70-260, 70-250, 70-240, 70-230, 70-220, 70-210, 70-200, 70-190, 70-180, 70-170, 70-160, 70-150, 70-140, 70-130, 70-120, 70-110, 70-100, 70-90, 70-80, 80-300, 80-290, 80-280, 80-270, 80-260, 80-250, 80-240, 80-230, 80-220, 80-210, 80-200, 80-190, 80-180, 80-170, 80-160, 80-150, 80-140, 80-130, 80-120, 80-110, 80-100, 80-90, 90-300, 90-290, 90-280, 90-270, 90-260, 90-250, 90-240, 90-230, 90-220, 90-210, 90-200, 90-190, 90-180, 90-170, 90-160, 90-150, 90-140, 90-130, 90-120, 90-110, or 90-100. In some embodiments, the integral b is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, or 300.

### Lipid Component

In some embodiments, the lipid component described herein includes an ionizable and/or permanently charged cationic lipid (e.g., any of the ionizable and/or permanently charged cationic lipids described herein), a helper lipid (e.g., any of the helper lipids described herein), a structural lipid (e.g., any of the structural lipids described herein), and/or a PEG lipid (e.g., any of the PEG lipids described herein).

Ionizable and/or permanently charged cationic lipids Ionizable lipids are positively charged at acidic pH to condense RNAs into LNPs, but are neutral at physiological pH to minimize toxicity. They can be protonated in the acidic endosome after cellular uptake, and interact with anionic endosomal phospholipids to form cone-shaped ion pairs that are not compatible with a bilayer. These cationic-anionic lipid pairs drive the transition from the bilayer structure to the inverted hexagonal Hu phase, which facilitates membrane fusion/disruption, endosomal escape and cargo release into the cytosol.

In some embodiments, the ionizable lipid described herein is an unsaturated ionizable lipid (e.g., DLin-MC3-DMA (MC3) or A18-Iso5-2DC18), a multi-tail ionizable lipid (e.g., 98N₁₂-5, C12-200, cKK-E12, or 9A1P9), an ionizable polymer-lipid (e.g., 7C1 or G0-C14), a biodegradable ionizable lipid (e.g., MC3, L319, 304O₁₃, C12-200, OF-Deg-Lin, OF-02, 306-O12B), or a branched-tailed ionizable lipid (e.g., 306Oᵢ₁₀ or FTT5). Details of ionizable lipids can be found, e.g., in Han, X., et al. “An ionizable lipid toolbox for RNA delivery.” Nature Communications 12.1 (2021): 7233; and Hou, X., et al. “Lipid nanoparticles for mRNA delivery.” Nature Reviews Materials 6.12 (2021): 1078-1094; each of which is incorporated herein by reference in its entirety.

In some embodiments, the ionizable and/or permanently charged cationic lipid described herein is an ionizable lipid (e.g., in the physiological pH range). For example, the ionizable cationic lipid can be selected from: [(4-hydroxybutyl)azanediyl]di(hexane-6,1-diyl) bis(2-hexyldecanoate) (ALC-0315); 9-Heptadecanyl 8-{(2-hydroxyethyl)[6-oxo-6-(undecyloxy)hexyl]amino}octanoate (SM-102); 1,2-dilinoleyloxy-N,N-dimethylaminopropane (DLin-DMA); heptatriaconta-6, 9,28,31-tetraen-19-yl 4-(dimethylamino)butanoate (DLin-MC3-DMA); 2,2-dilinoleyl-4-(2-dimethylaminoethyl)-[1, 3]-dioxolane (DLin-KC2-DMA); 2,2-dilinoleyl-4-dimethylaminomethyl-[1,3]-dioxolane (DLin-K-DMA); N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25); 1,2-dioleyloxy-N,N-dimethylaminopropane (DODMA); 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA); (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)); and/or (2S)-2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).

In some embodiments, the ionizable and/or permanently charged cationic lipid described herein is a permanently charged cationic lipid (e.g., in the physiological pH range). In some embodiments, such lipid is also called fixed cationic lipid. Fixed cationic lipids are permanently positively charged regardless of the pH of its biological environment. Cationic lipids are similar to other natural lipids except for the replacement of a typical headgroup with a cationic headgroup. Fixed cationic lipids have been proven to be good transfection agents in cationic liposomes (CLs) and LNPs. Generally considered non-toxic at lower concentrations, these lipids do present some toxicity concerns when used in higher concentrations due to the tetrasubstituted ammonium moiety.

Fixed cationic lipids are generally cheaper, more readily available alternatives to ionizable lipids. Historically, fixed cationic lipids have been more widely studied and therefore a large amount of data has been generated to support their use. They are commonly used for the delivery of DNA, as well as the delivery of siRNA and saRNA where a lesser payload is needed.

In some embodiments, the permanently charged cationic lipid can be Dioleoyl-3-trimethylammonium propane (DOTAP); 1,2-di-O-octadecenyl-3-trimethylammonium-propane (DOTMA); Dimethyldioctadecylammonium bromide (DDAB); 2,3-dioleyloxy-N-[2-(sperminecarboxamido)ethyl]-N,N-dimethyl-1-propanaminium trifluoroacetate (DOSPA); 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide (BHEM-Cholesterol); and/or Ethylphosphatidylcholine (ePC). Details of such cationic lipids can be found, e.g., in Hou, X., et al. “Lipid nanoparticles for mRNA delivery.” Nature Reviews Materials 6.12 (2021): 1078-1094, which is incorporated herein by reference in its entirety.

In some embodiments, the ionizable and/or permanently charged cationic lipid described herein is N,N-dioleyl-N,N-dimethylammonium chloride (“DODAC”); N-(2,3-dioleyloxy)propyl)-N,N,N-trimethylammonium chloride (“DOTMA”); N-(2,3-dioleyloxy)propyl)-N,N-dimethylammonium chloride (“DODMA”); N-(2,3-dioleoyloxy)propyl)-N,N-dimethylammonium chloride (“DODAP”); 3-(N—(N′,N′-dimethylaminoethane)carbamoyl)cholesterol (“DC-Chol”); N-(1,2-dimyristyloxyprop-3-yl)-N,N-dimethyl-N-hydroxyethyl ammonium bromide (“DMR1E”); 1,2-dilinoleyloxy-N,N-dimethyl-3-aminopropane (DLinDMA); 1,2-distearyloxy-N,N-dimethyl-3-aminopropane (DSDMA); 1,2-dilinolenyloxy-N,N-dimethyl-3-aminopropane (DLenDMA); or 2-{4-[(3b)-cholest-5-en-3-yloxy]butoxy}-N,N-dimethyl-3-[(9Z,12Z)-octadeca-9,12-dien-1-yloxy]propan-amine (CLinDMA), 3β-[N-(Dimethylaminoethane)-carbamoyl]-cholesterol (DC-Chol).

### Helper Lipids

Helper lipids are typically included as LNP components to provide particle stability, blood compatibility, and to enhance cargo delivery efficiency. Helper lipids with cone-shape geometry favoring the formation hexagonal II phase, such as DOPE, can promote endosomal release of cargo molecules. Meanwhile, cylindrical-shaped lipid phosphatidylcholine can provide greater bilayer stability, which is important for in vivo application of LNPs. Details can be found, e.g., in Cheng, X., et al. “The role of helper lipids in lipid nanoparticles (LNPs) designed for oligonucleotide delivery.” Advanced Drug Delivery Reviews 99 (2016): 129-137, which is incorporated herein by reference in its entirety.

In some embodiments, the helper lipid described herein in a phospholipid. In some embodiments, the phospholipid comprises a moiety selected from the group consisting of phosphatidyl choline, phosphatidyl ethanolamine, phosphatidyl glycerol, phosphatidyl serine, phosphatidic acid, 2-lysophosphatidyl choline, and a sphingomyelin. In some embodiments, the phospholipid comprises one or more fatty acid moieties selected from lauric acid, myristic acid, myristoleic acid, palmitic acid, palmitoleic acid, stearic acid, oleic acid, linoleic acid, alpha-linolenic acid, erucic acid, phytanoic, arachidic acid, arachidonic acid, eicosapentaenoic acid, behenic acid, docosapentaenoic acid, and docosahexaenoic acid.

In some embodiments, the phospholipid is selected from 1,2-distearoyl-sn-glycero-3-phosphocholine (DSPC); 1,2-dioleoyl-sn-glycero-3-phosphoethanola mine (DOPE); 1,2-dilinoleoyl-sn-glycero-3-phosphocholine (DLPC); 1,2-dimyristoyl-sn-glycero-phosphocholine (DMPC); 1,2-dioleoyl-sn-glycero-3-phosphocholine (DOPC); 1,2-dipalmitoyl-sn-glycero-3-phosphocholine (DPPC); 1,2-diundecanoyl-sn-glycero-phosphocholine (DUPC); 1-palmitoyl-2-oleoyl-sn-glycero-3-phosphocholine (POPC); 1,2-di-0-octadecenyl-sn-glycero-3-phosphocholine (1 8:0 Diether PC); 1-oleoyl-2-cholesterylhemisuccinoyl-sn-glycero-3-phosphocholine (OChemsPC); 1-hexadecyl-sn-glycero-3-phosphocholine (C16 Lyso PC); 1,2-dilinolenoyl-sn-glycero-3-phosphocholine, 1, 2-diarachidonoyl-sn-glycero-3-phosphocholine; 1,2-didocosahexaenoyl-sn-glycero-3-phosphocholine; 1,2-diphytanoyl-sn-glycero-3-phosphoethanolamine (ME 16.0 PE); 1,2-distearoyl-sn-glycero-3-phosphoethanolamine; 1,2-dilinoleoyl-sn-glycero-3-phosphoethanolamine; 1,2-dilinolenoyl-sn-glycero-3-phosphoethanolamine; 1,2-diarachidonoyl-sn-glycero-3-phosphoethanolamine; 1,2-didocosahexaenoyl-sn-glycero-3-phosphoethanolamine; and/or 1,2-dioleoyl-sn-glycero-3-phospho-rac-(1-glycerol) sodium salt (DOPG), and sphingomyelin. In some embodiments, the lipid component includes both DOPE and DSPC.

In some embodiments, the helper lipid described herein is a non-cationic lipid. In some embodiments, the helper lipid is a saturated lipid, a non-saturated lipid, or a combination of a saturated lipid and a non-saturated lipid.

In some embodiments, the helper lipid described herein is an anionic lipid, e.g., diacylglycerol phophatidic acid (1,2-distearoyl-sn-glycero-3-phosphate (DSPA); 1,2-dipalmitoyl-sn-glycero-3-phosphate (DPPA); 1,2-dimyristoyl-sn-glycero-3-phosphate (DMPA); 1,2-dilauroyl-sn-glycero-3-phosphate (DLPA); 1,2-dioleoyl-sn-glycero-3-phosphate (DOPA)), diacylglycerol phosphoglycerol (1,2-distearoyl-sn-glycero-3-phospho-(1′-rac-glycerol) (DSPG); 1,2-dipalmitoyl-sn-glycero-3-phospho-(1′-rac-glycerol) (DPPG); 1,2-dimyristoyl-sn-glycero-3-phospho-(1′-rac-glycerol) (DMPG); 1,2-dilauroyl-sn-glycero-3-phospho-(1′-rac-glycerol) (DLPG); 1,2-dioleoyl-sn-glycero-3-phospho-(1′-rac-glycerol) (DOPG)), phosphatidylglycerol, cardiolipin, diacylphosphatidylserine, N-succinyl phosphatidylethanolamines, N-glutarylphosphatidylethanolamines, lysylphosphatidylglycerols, and other anionic modifying groups joined to neutral lipids. The mixture of lipids may also include a neutral lipid. The neutral lipids may be but are not limited to diacylglycerol phosphocholine (L-α-phosphatidylcholine, hydrogenated (Soy) (HSPC); 1,2-distearoyl-sn-glycero-3-phosphocholine (DSPC); 1,2-dipalmitoyl-sn-glycero-3-phosphocholine (DPPC); 1,2-dimyristoyl-sn-glycero-3-phosphocholine (DMPC); 1,2-dilauroyl-sn-glycero-3-phosphocholine (DLPC); 1,2-dioleoyl-sn-glycero-3-phosphocholine (DOPC), diacylglycerol phosphoethanolamine (1,2-distearoyl-sn-glycero-3-phosphoethanolamine (DSPE); 1,2-dipalmitoyl-sn-glycero-3-phosphoethanolamine (DPPE); 1,2-dimyristoyl-sn-glycero-3-phosphoethanolamine (DMPE); 1,2-dilauroyl-sn-glycero-3-phosphoethanolamine (DLPE); 1,2-dioleoyl-sn-glycero-3-phosphoethanolamine (DOPE), and phosphatidylserine.

### Structural Lipids

The lipid component of a nanoparticle composition may include one or more structural lipids. In some embodiments, the structural lipid described herein is selected from cholesterol, fecosterol, sitosterol, ergosterol, campesterol, stigmasterol, brassicasterol, tomatidine, ursolic acid, and alpha-tocopherol. In some embodiments, the structural lipid is cholesterol.

The role of cholesterol in membranes largely depends on context. When combined with phospholipids with low gel-liquid crystalline phase transitions (Tₘ), cholesterol helps formation of the liquid-ordered phase which is characterized by decreased membrane fluidity and increased bilayer thickness. Cholesterol and low Tₘ lipids undergo a “condensation” whereby the cross-sectional area of the lipid and cholesterol is lower than the sum of the individual cross-sectional areas. However, when combined with high Tₘ lipids, cholesterol boosts membrane fluidity and narrows the bilayer. In both cases, cholesterol pulls the lipids towards a liquid-ordered phase.

In LNP formulations containing nucleic acid, incorporating cholesterol is largely based on two main findings obtained with liposomal formulations of small molecule therapeutics: 1) cholesterol is an exchangeable molecule and can accumulate within a liposome during circulation, and 2) cholesterol dramatically reduces the amount of surface-bound protein and improves circulation half-lives. Therefore, an equimolar amount of cholesterol is included in LNP formulations relative to endogenous membranes; this prevents net efflux or influx and maintains membrane integrity. Further, it was found to be essential to particle stability and subsequently debuted in stable antisense lipid particles (SALP).

Structural lipids such as cholesterol are also essential for encapsulating nucleic acid. As cholesterol increase membrane rigidity, it serves to reduce drug leakage from the liposomal core. This effect may however not be considered important for large cargo such as nucleic acids. Details can be found, e.g., in Albertsen, C. H., et al. “The role of lipid components in lipid nanoparticles for vaccines and gene therapy.” Advanced Drug Delivery Reviews (2022): 114416, which is incorporated herein by reference in its entirety.

### PEG Lipids (or PEGylated Lipid)

The lipid component of a nanoparticle composition of the disclosure may include one or more PEG or PEG-modified lipids. Such species may be alternately referred to as PEGylated lipids. A PEG lipid is a lipid modified with polyethylene glycol.

The lipid component of a nanoparticle composition may include one or more PEG lipids. Even though PEG lipids constitute the smallest molar percentage of the lipid components in LNPs (typically about 1.5 mol %), they influence several key properties thereof: population size and dispersity; LNP aggregation prevention; and particle stability during both preparation and storage. Furthermore, PEG lipids also affect factors such as nucleic acid encapsulation efficiency; circulation half-life; in vivo distribution; transfection efficiency; and immune response. All these properties are somewhat related to the molar ratio of the PEG lipid as well as the structure and length of both the PEG chain and the lipid tail (alkyl/dialkyl chain(s)).

In some embodiments, the PEG lipid described herein is selected from a PEG-modified phosphatidylethanolamine, a PEG-modified phosphatide acid, a PEG-modified ceramide, a PEG-modified diaikylamine, a PEG-modified diacylglycerol, and a PEG-modified dialkylglycerol. In some embodiments, the PEG lipid is 1,2-Distearoyl-sn-Glycero-3-Phosphoethanolamine-N-[Methoxy(Polyethylene glycol)-2000] (PEG2000-DSPE) or 1,2-dimyristoyl-rac-glycero-3-methoxypolyethylene glycol-2000 (PEG2000-DMG).

In some embodiments, the PEG lipid may be but are not limited to 1,2-distearoyl-sn-glycero-3-phosphoethanolamine-N-[methoxy(polyethylene glycol)-2000] (mPEG-2000-DSPE); 1,2-dioctadecanoyl-sn-glycero-3-phosphoethanolamine-N-[methoxy(polyethylene glycol)-2000] (mPEG-2000-DOPE); 1,2-dipalmitoyl-sn-glycero-3-phosphoethanolamine-N-[methoxy(polyethylene glycol)-2000] (mPEG-2000-DPPE); 1,2-dimyristoyl-sn-glycero-3-phosphoethanolamine-N-[methoxy(polyethylene glycol)-2000] (mPEG-2000-DMPE); 1,2-dilauroyl-sn-glycero-3-phosphoethanolamine-N-[methoxy(polyethylene glycol)-2000](mPEG-2000-DLPE); 1,2-distearoyl-sn-glycero-3-phosphoethanolamine-N-[methoxy(polyethylene glycol)-5000] (mPEG-5000-DSPE); 1,2-dioctadecanoyl-sn-glycero-3-phosphoethanolamine-N-[methoxy(polyethylene glycol)-5000] (mPEG-5000-DOPE); 1,2-dipalmitoyl-sn-glycero-3-phosphoethanolamine-N-[methoxy(polyethylene glycol)-5000](mPEG-5000-DPPE); 1,2-dimyristoyl-sn-glycero-3-phosphoethanolamine-N-[methoxy(polyethylene glycol)-5000] (mPEG-5000-DMPE); 1,2-dilauroyl-sn-glycero-3-phosphoethanolamine-N-[methoxy(polyethylene glycol)-5000] (mPEG-5000-DLPE).

In some embodiments, the PEG lipid has an average molecular weight of about 500-5000 Daltons, about 500-4500 Daltons, about 500-4000 Daltons, about 500-3500 Daltons, about 500-3000 Daltons, about 500-2500 Daltons, about 500-2000 Daltons, about 500-1500 Daltons, about 500-1000 Daltons, about 1000-5000 Daltons, about 1000-4500 Daltons, about 1000-4000 Daltons, about 1000-3500 Daltons, about 1000-3000 Daltons, about 1000-2500 Daltons, about 1000-2000 Daltons, about 1000-1500 Daltons, about 1500-5000 Daltons, about 1500-4500 Daltons, about 1500-4000 Daltons, about 1500-3500 Daltons, about 1500-3000 Daltons, about 1500-2500 Daltons, about 1500-2000 Daltons, about 2000-5000 Daltons, about 2000-4500 Daltons, about 2000-4000 Daltons, about 2000-3500 Daltons, about 2000-3000 Daltons, about 2000-2500 Daltons, about 2500-5000 Daltons, about 2500-4500 Daltons, about 2500-4000 Daltons, about 2500-3500 Daltons, about 2500-3000 Daltons, about 3000-5000 Daltons, about 3000-4500 Daltons, about 3000-4000 Daltons, about 3000-3500 Daltons, about 3500-5000 Daltons, about 3500-4500 Daltons, about 3500-4000 Daltons, about 4000-5000 Daltons, about 4000-4500 Daltons, or about 4500-5000 Daltons.

### Cargo Molecules

Also provided herein are cargo molecules of the LNPs described herein. In some embodiments, the cargo molecule is a nucleic acid.

In some embodiments, the nucleic acid described herein is an RNA, e.g., small interfering RNA (siRNA), microRNA (miRNA), messenger mRNA (mRNA), guide RNA (gRNA), circular RNA (circRNA), self-amplifying RNA (saRNA), or an antisense RNA thereof. In some embodiments, the nucleic acid described herein is a DNA, e.g., double stranded DNA (dsDNA), plasmid DNA, single stranded DNA (ssDNA), or an antisense DNA thereof.

Examples of nucleic acid include DNA such as genomic DNA, methylated DNA, specific methylated DNA sequences, fragmented DNA, mitochondrial DNA, in situ synthesized PCR products, and RNA/DNA hybrids.

Examples of nucleic acid also include RNA such as various types of coding and non-coding RNA. Examples of the different types of RNA include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments, the nucleic acid is capable of functioning as a component of a gene editing reaction, such as, for example, clustered regularly interspaced short palindromic repeats (CRISPR)-based gene editing.

### Other Components

A nanoparticle composition may include one or more components in addition to those described in the preceding sections. For example, a nanoparticle composition may include one or more small hydrophobic molecules such as a vitamin (e.g., vitamin A or vitamin E) or a sterol. Nanoparticle compositions may also include one or more permeability enhancer molecules, carbohydrates, polymers, therapeutic agents, surface altering agents, or other components. A permeability enhancer molecule may be a molecule described by U.S. Patent Application Publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a nanoparticle composition. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as polyvinyl acetate), polyvinyl halides such as polyvinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, polyoxamines, poly(ortho)esters, poly(butyric acid), poly(valeric acid), poly(lactide-co-caprolactone), and trimethylene carbonate, polyvinylpyrrolidone.

Therapeutic agents may include, but are not limited to, cytotoxic, chemotherapeutic, and other therapeutic agents. Cytotoxic agents may include, for example, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, rachelmycin, and analogs thereof. Radioactive ions may also be used as therapeutic agents and may include, for example, radioactive iodine, strontium, phosphorous, palladium, cesium, iridium, cobalt, yttrium, samarium, and praseodymium. Other therapeutic agents may include, for example, antimetabolites (e.g., methotrexate, 6-mercaptopurine, 6-thioguanine, cytarabine, and 5-fluorouracil, and decarbazine), alkylating agents (e.g., mechlorethamine, thiotepa, chlorambucil, rachelmycin, melphalan, carmustine, lomustine, cyclophosphamide, busulfan, dibromomannitol, streptozotocin, mitomycin C, and cis-dichlorodiamine platinum (II) (DDP), and cisplatin), anthracyclines (e.g., daunorubicin and doxorubicin), antibiotics (e.g., dactinomycin, bleomycin, mithramycin, and anthramycin), and anti-mitotic agents (e.g., vincristine, vinblastine, taxol, and maytansinoids).

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, domase alfa, neltenexine, and erdosteine), and DNases (e.g., rhDNase). A surface altering agent may be disposed within a nanoparticle and/or on the surface of a nanoparticle composition (e.g., by coating, adsorption, covalent linkage, or other process).

In addition to these components, nanoparticle compositions of the disclosure may include any substance useful in pharmaceutical compositions. For example, the nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21^{st} Edition, A. R. Gennaro; Lippincott, Williams & Wilkins, Baltimore, M D, 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER® 188, cetrimonium bromide, cetylpyridinium chloride, benzalkonium chloride, docusate sodium, and/or combinations thereof.

A binding agent may be starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite.

Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g. HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, camauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### Compositions of PALNPs

In some embodiments, the nanoparticle composition described herein includes: (a) about 0.1 mol % to about 20 mol % of the compound of formula I, formula II, formula III, formula IV, or a derivative compound thereof; (b) about 5 mol % to about 30 mol % of the ionizable and/or permanently charged cationic lipid (e.g., ALC-0315, SM-102, DLin-DMA, DLin-MC3-DMA, and/or DLin-KC2-DMA); (c) about 10 mol % to about 50 mol % of the helper lipid (e.g., DSPC and/or DOPE); (d) about 20 mol % to about 50 mol % of the structural lipid (e.g., cholesterol); and (e) about 0.5 mol % to about 5 mol % of the PEG lipid (e.g., PEG2000-DSPE).

As used herein, the term “mol %” stands for the molar ratio of one or more compounds relative to the total molar amount of the nanoparticle composition described herein (e.g., a nanoparticle composition including the lipid component and the polymer component described herein). In some embodiments, the “mol %” of a certain compound is calculated without considering the molar amount of the nucleic acid encapsulated in the lipid nanoparticle.

In some embodiments, the compound of formula I, formula II, formula III, or formula IV accounts for about 1 mol % to about 10 mol % (e.g., about 1 mol % to about 9 mol %, about 1 mol % to about 8 mol %, about 1 mol % to about 7 mol %, about 1 mol % to about 6 mol %, about 1 mol % to about 5 mol %, about 1 mol % to about 4 mol %, about 1 mol % to about 3 mol %, about 1 mol % to about 2 mol %, about 2 mol % to about 10 mol %, about 2 mol % to about 9 mol %, about 2 mol % to about 8 mol %, about 2 mol % to about 7 mol %, about 2 mol % to about 6 mol %, about 2 mol % to about 5 mol %, about 2 mol % to about 4 mol %, about 2 mol % to about 3 mol %, about 3 mol % to about 10 mol %, about 3 mol % to about 9 mol %, about 3 mol % to about 8 mol %, about 3 mol % to about 7 mol %, about 3 mol % to about 6 mol %, about 3 mol % to about 5 mol %, about 3 mol % to about 4 mol %, about 4 mol % to about 10 mol %, about 4 mol % to about 9 mol %, about 4 mol % to about 8 mol %, about 4 mol % to about 7 mol %, about 4 mol % to about 6 mol %, about 4 mol % to about 5 mol %, about 5 mol % to about 10 mol %, about 5 mol % to about 9 mol %, about 5 mol % to about 8 mol %, about 5 mol % to about 7 mol %, about 5 mol % to about 6 mol %, about 6 mol % to about 10 mol %, about 6 mol % to about 9 mol %, about 6 mol % to about 8 mol %, about 6 mol % to about 7 mol %, about 7 mol % to about 10 mol %, about 7 mol % to about 9 mol %, about 7 mol % to about 8 mol %, about 8 mol % to about 10 mol %, about 8 mol % to about 9 mol %, or about 9 mol % to about 10 mol %) of the nanoparticle composition. In some embodiments, the formula I is L121, which accounts for about 5 mol % to about 7 mol % (e.g., about 5, about 5.1, about 5.2, about 5.3, about 5.4, about 5.5, about 5.6, about 5.7. about 5.8. about 5.9, about 6, about 6.1, about 6.2, about 6.3, about 6.4, about 6.5, about 6.6, about 6.7, about 6.8, about 6.9, or about 7 mol %) of the nanoparticle composition. In some embodiments, the compound of formula I is L92, which accounts for about 4 mol % to about mol % (e.g., about 4, about 4.1, about 4.2, about 4.3, about 4.4, about 4.5, about 4.6, about 4.7, about 4.8, about 4.9, or about 5 mol %) of the nanoparticle composition. In some embodiments, the compound of formula I is L81, which accounts for about 2 mol % to about 4 mol % (e.g., about 2, about 2.1, about 2.2, about 2.3, about 2.4, about 2.5, about 2.6, about 2.7, about 2.8, about 2.9. about 3, about 3.1, about 3.2, about 3.3, about 3.4, about 3.5, about 3.6, about 3.7, about 3.8, about 3.9, or about 4) of the nanoparticle composition.

In some embodiments, the ionizable and/or permanently charged cationic lipid described herein accounts for about 5 mol % to about 30 mol %, about 5 mol % to about 25 mol %, about 5 mol % to about 20 mol %, about 5 mol % to about 15 mol %, about 5 mol % to about 10 mol %, about 10 mol % to about 30 mol %, about 10 mol % to about 25 mol %, about mol % to about 20 mol %, about 10 mol % to about 15 mol %, about 15 mol % to about 30 mol %, about 15 mol % to about 25 mol %, about 15 mol % to about 20 mol %, about 20 mol % to about 30 mol %, about 20 mol % to about 25 mol %, or about 25 mol % to about 30 mol % of the nanoparticle composition. In some embodiments, the ionizable and/or permanently charged cationic lipid described herein accounts for about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% of the nanoparticle composition.

In some embodiments, the ionizable and/or permanently charged cationic lipid within the LNPs (e.g., PALNPs) or LNPs having the nanoparticle compositions described herein is less than 20%, less than 10%, less than 5%, less than 4%, less than 3%, less than 2%, less than 1%, less than 0.5%, less than 0.4%, less than 0.3%, less than 0.2%, or less than 0.1% of the ionizable and/or permanently charged cationic lipid used in a reference LNP, e.g., a LNP that does not include the polymer component (e.g., any of the polymer components described herein), e.g., LNPs used as COVID-10 vaccines described herein.

In some embodiments, the helper lipid described herein accounts for about 10 mol % to about 50 mol %, about 10 mol % to about 45 mol %, about 10 mol % to about 40 mol %, about 10 mol % to about 35 mol %, about 10 mol % to about 30 mol %, about 10 mol % to about mol %, about 10 mol % to about 20 mol %, about 10 mol % to about 15 mol %, about 15 mol % to about 50 mol %, about 15 mol % to about 45 mol %, about 15 mol % to about 40 mol %, about 15 mol % to about 35 mol %, about 15 mol % to about 30 mol %, about 15 mol % to about 25 mol %, about 15 mol % to about 20 mol %, about 20 mol % to about 50 mol %, about 20 mol % to about 45 mol %, about 20 mol % to about 40 mol %, about 20 mol % to about mol %, about 20 mol % to about 30 mol %, about 20 mol % to about 25 mol %, about 25 mol % to about 50 mol %, about 25 mol % to about 45 mol %, about 25 mol % to about 40 mol %, about 25 mol % to about 35 mol %, about 25 mol % to about 30 mol %, about 30 mol % to about 50 mol %, about 30 mol % to about 45 mol %, about 30 mol % to about 40 mol %, about 30 mol % to about 35 mol %, about 35 mol % to about 50 mol %, about 35 mol % to about mol %, about 35 mol % to about 40 mol %, about 40 mol % to about 50 mol %, about 40 mol % to about 45 mol %, or about 45 mol % to about 50 mol % of the nanoparticle composition.

In some embodiments, the helper lipid described herein includes about 5 mol % to about 30 mol % of DSPC or HSPC, and about 5 mol % to about 30 mol % of DOPE.

In some embodiments, the helper lipid described herein includes about 10 mol % to about 30 mol % (e.g., about 10 mol % to about 28 mol %, about 10 mol % to about 26 mol %, about 10 mol % to about 24 mol %, about 10 mol % to about 22 mol %, about 10 mol % to about mol %, about 10 mol % to about 18 mol/q, about 10 mol % to about 16 mol/q, about 10 mol % to about 14 mol %, about 10 mol % to about 12 mol %, about 12 mol % to about 30 mol %, about 12 mol % to about 28 mol %, about 12 mol % to about 16 mol %, about 12 mol % to about 24 mol %, about 12 mol % to about 22 mol %, about 12 mol % to about 20 mol %, about 12 mol % to about 18 mol %, about 12 mol % to about 16 mol %, about 12 mol % to about 14 mol %, about 14 mol % to about 30 mol %, about 14 mol % to about 28 mol %, about 14 mol % to about 26 mol %, about 14 mol % to about 24 mol %, about 14 mol % to about 22 mol %, about 14 mol % to about 20 mol %, about 14 mol % to about 18 mol %, about 14 mol % to about 16 mol %, about 16 mol % to about 30 mol %, about 16 mol % to about 28 mol %, about 16 mol % to about 26 mol %, about 16 mol % to about 24 mol %, about 16 mol % to about 22 mol %, about 16 mol % to about 20 mol %, about 16 mol % to about 18 mol %, about 18 mol % to about 30 mol %, about 18 mol % to about 28 mol %, about 18 mol % to about 26 mol %, about 18 mol % to about 24 mol %, about 18 mol % to about 22 mol %, about 18 mol % to about 20 mol %, about 20 mol % to about 30 mol %, about 20 mol % to about 28 mol %, about 20 mol % to about 26 mol %, about 20 mol % to about 24 mol %, about 20 mol % to about 22 mol %, about 22 mol % to about 30 mol %, about 22 mol % to about 28 mol %, about 22 mol % to about 26 mol %, about 22 mol % to about 24 mol %, about 24 mol % to about 30 mol %, about 24 mol % to about 28 mol %, about 24 mol % to about 26 mol %, about 26 mol % to about 30 mol %, about 26 mol % to about 28 mol %, or about 28 mol % to about 30 mol %) of DSPC or a derivative thereof, and about 10 mol % to about 30 mol % (e.g., about 10 mol % to about 28 mol %, about 10 mol % to about 26 mol %, about 10 mol % to about 24 mol %, about 10 mol % to about 22 mol %, about 10 mol % to about 20 mol %, about 10 mol % to about 18 mol %, about 10 mol % to about 16 mol %, about 10 mol % to about 14 mol %, about 10 mol % to about 12 mol %, about 12 mol % to about 30 mol %, about 12 mol % to about 28 mol %, about 12 mol % to about 16 mol %, about 12 mol % to about 24 mol %, about 12 mol % to about 22 mol %, about 12 mol % to about 20 mol %, about 12 mol % to about 18 mol %, about 12 mol % to about 16 mol %, about 12 mol % to about 14 mol %, about 14 mol % to about mol %, about 14 mol % to about 28 mol %, about 14 mol % to about 26 mol %, about 14 mol % to about 24 mol %, about 14 mol % to about 22 mol %, about 14 mol % to about 20 mol %, about 14 mol % to about 18 mol %, about 14 mol % to about 16 mol %, about 16 mol % to about 30 mol %, about 16 mol % to about 28 mol %, about 16 mol % to about 26 mol %, about 16 mol % to about 24 mol %, about 16 mol % to about 22 mol %, about 16 mol % to about mol %, about 16 mol % to about 18 mol %, about 18 mol % to about 30 mol %, about 18 mol % to about 28 mol %, about 18 mol % to about 26 mol %, about 18 mol % to about 24 mol %, about 18 mol % to about 22 mol %, about 18 mol % to about 20 mol %, about 20 mol % to about 30 mol %, about 20 mol % to about 28 mol %, about 20 mol % to about 26 mol %, about 20 mol % to about 24 mol %, about 20 mol % to about 22 mol %, about 22 mol % to about mol %, about 22 mol % to about 28 mol %, about 22 mol % to about 26 mol %, about 22 mol % to about 24 mol %, about 24 mol % to about 30 mol %, about 24 mol % to about 28 mol %, about 24 mol % to about 26 mol %, about 26 mol % to about 30 mol %, about 26 mol % to about 28 mol %, or about 28 mol % to about 30 mol %) of DOPE or a derivative thereof.

In some embodiments, the structural lipid described herein accounts for about 20 mol % to about 50 mol %, about 20 mol % to about 45 mol %, about 20 mol % to about 40 mol %, about 20 mol % to about 35 mol %, about 20 mol % to about 30 mol %, about 20 mol % to about 25 mol %, about 25 mol % to about 50 mol %, about 25 mol % to about 45 mol %, about 25 mol % to about 40 mol %, about 25 mol % to about 35 mol %, about 30 mol % to about 50 mol %, about 30 mol % to about 45 mol %, about 30 mol % to about 40 mol %, about 30 mol % to about 45 mol %, about 35 mol % to about 50 mol %, about 35 mol % to about 45 mol %, about 35 mol % to about 40 mol %, about 40 mol % to about 50 mol %, about 40 mol % to about 45 mol %, or about 45 mol % to about 50 mol % of the nanoparticle composition.

In some embodiments, the PEG lipid described herein accounts for about 0.5 mol % to about 5 mol %, about 0.5 mol % to about 4.5 mol %, about 0.5 mol % to about 4 mol %, about 0.5 mol % to about 3.5 mol %, about 0.5 mol % to about 3 mol %, about 0.5 mol % to about 2.5 mol %, about 0.5 mol % to about 2 mol %, about 0.5 mol % to about 1.5 mol %, about 0.5 mol % to about 1 mol %, about 1 mol % to about 5 mol %, about 1 mol % to about 4.5 mol %, about 1 mol % to about 4 mol %, about 1 mol % to about 3.5 mol %, about 1 mol % to about 3 mol %, about 1 mol % to about 2.5 mol %, about 1 mol % to about 2 mol %, about 1 mol % to about 1.5 mol %, about 1.5 mol % to about 5 mol %, about 1.5 mol % to about 4.5 mol %, about 1.5 mol % to about 4 mol %, about 1.5 mol % to about 3.5 mol %, about 1.5 mol % to about 3 mol %, about 1.5 mol % to about 2.5 mol %, about 1.5 mol % to about 2 mol %, about 2 mol % to about 5 mol %, about 2 mol % to about 4.5 mol %, about 2 mol % to about 4 mol %, about 2 mol % to about 3.5 mol %, about 2 mol % to about 3 mol %, about 2 mol % to about 2.5 mol %, about 2.5 mol % to about 5 mol %, about 2.5 mol % to about 4.5 mol %, about 2.5 mol % to about 4 mol %, about 2.5 mol % to about 3.5 mol %, about 2.5 mol % to about 3 mol %, about 3 mol % to about 5 mol %, about 3 mol % to about 4.5 mol %, about 3 mol % to about 4 mol %, about 3 mol % to about 3.5 mol %, about 3.5 mol % to about 5 mol %, about 3.5 mol % to about 4.5 mol %, about 3.5 mol % to about 4 mol %, about 4 mol % to about 5 mol %, about 4 mol % to about 4.5 mol %, or about 4.5 mol % to about 5 mol % of the nanoparticle composition.

In some embodiments, provided herein is CPT067 or LNPs having a substantially similar formulation of CPT067, that includes: (a) about 0.1 mol % to about 10 mol % (e.g., about 5 mol %) of the compound of the copolymer described herein (e.g., L121); (b) about 30 mol % to about 50 mol % (e.g., about 41.5 mol %) of DLin-DMA or a derivative thereof; (c) about 5 mol % to about 20 mol % (e.g., about 10 mol %) of DSPC or a derivative thereof; (d) about 20 mol % to about 50 mol % (e.g., about 38.5 mol %) of cholesterol or a derivative thereof; and (e) about 0.5 mol % to about 5 mol % (e.g., about 1.5 mol %) of PEG2000-DSPE or a derivative thereof.

In some embodiments, provided herein is CPT069 or LNPs having a substantially similar formulation of CPT069, that includes: (a) about 0.1 mol % to about 10 mol % (e.g., about 5 mol %) of the compound of the copolymer described herein (e.g., L121); (b) about 30 mol % to about 50 mol % (e.g., about 41.5 mol %) of DLin-KC2-DMA or a derivative thereof; (c) about 5 mol % to about 20 mol % (e.g., about 10 mol %) of DSPC or a derivative thereof; (d) about 20 mol % to about 50 mol % (e.g., about 41.5 mol %) of cholesterol or a derivative thereof; and (e) about 0.5 mol % to about 5 mol % (e.g., about 2 mol %) of PEG2000-DSPE or a derivative thereof.

In some embodiments, provided herein is CPT104 or LNPs having a substantially similar formulation of CPT104, that includes: (a) about 0.1 mol % to about 10 mol % (e.g., about 5 mol %) of the compound of the copolymer described herein (e.g., L121); (b) about 30 mol % to about 60 mol % (e.g., about 45 mol %) of DLin-KC2-DMA or a derivative thereof; (c) about 5 mol % to about 20 mol % (e.g., about 10 mol %) of DSPC or a derivative thereof; (d) about 20 mol % to about 50 mol % (e.g., about 38.5 mol %) of cholesterol or a derivative thereof; and (e) about 0.5 mol % to about 5 mol % (e.g., about 1.5 mol %) of PEG2000-DSPE or a derivative thereof.

In some embodiments, provided herein is CPT106 or LNPs having a substantially similar formulation of CPT106, that includes: (a) about 0.1 mol % to about 10 mol % (e.g., about 5 mol %) of the compound of the copolymer described herein (e.g., L121); (b) about 30 mol % to about 60 mol % (e.g., about 45 mol %) of DLin-MC3-DMA or a derivative thereof; (c) about 5 mol % to about 20 mol % (e.g., about 10 mol %) of DSPC or a derivative thereof; (d) about 20 mol % to about 50 mol % (e.g., about 38.5 mol %) of cholesterol or a derivative thereof; and (e) about 0.5 mol % to about 5 mol % (e.g., about 1.5 mol %) of PEG2000-DSPE or a derivative thereof.

In some embodiments, provided herein is CPT108 or LNPs having a substantially similar formulation of CPT108, that includes: (a) about 0.1 mol % to about 10 mol % (e.g., about 5 mol %) of the compound of the copolymer described herein (e.g., L121); (b) about 30 mol % to about 60 mol % (e.g., about 45 mol %) of ALC-0315 or a derivative thereof; (c) about 5 mol % to about 20 mol % (e.g., about 10 mol %) of DSPC or a derivative thereof; (d) about 20 mol % to about 50 mol % (e.g., about 38.5 mol %) of cholesterol or a derivative thereof; and (e) about 0.5 mol % to about 5 mol % (e.g., about 1.5 mol %) of PEG2000-DSPE or a derivative thereof.

In some embodiments, provided herein is CPT147 or LNPs having a substantially similar formulation of CPT147, that includes: (a) about 0.1 mol % to about 10 mol % (e.g., about 5 mol %) of the compound of the copolymer described herein (e.g., L121); (b) about 1 mol % to about 20 mol % (e.g., about 10 mol %) of ALC-0315 or a derivative thereof; (c) about 10 mol % to about 30 mol % (e.g., about 22.5 mol %) of DSPC or a derivative thereof, and about 10 mol % to about 30 mol % (e.g., about 22.5 mol %) of DOPE or a derivative thereof; (d) about 20 mol % to about 50 mol % (e.g., about 38.5 mol %) of cholesterol or a derivative thereof; and (e) about 0.5 mol % to about 5 mol % (e.g., about 1.5 mol %) of PEG2000-DSPE or a derivative thereof.

In some embodiments, provided herein is CPT147-38 or LNPs having a substantially similar formulation of CPT147-38, that includes: (a) about 0.1 mol % to about 10 mol % (e.g., about 6.8 mol %) of the compound of the copolymer described herein (e.g., L121); (b) about mol % to about 30 mol % (e.g., about 18 mol %) of ALC-0315 or a derivative thereof; (c) about 10 mol % to about 30 mol % (e.g., about 20 mol %) of DSPC or a derivative thereof, and about 10 mol % to about 30 mol % (e.g., about 20 mol %) of DOPE or a derivative thereof; (d) about 20 mol % to about 50 mol % (e.g., about 34 mol %) of cholesterol or a derivative thereof; and (e) about 0.5 mol % to about 5 mol % (e.g., about 1.5 mol %) of PEG2000-DSPE or a derivative thereof.

In some embodiments, provided herein is CPT163-21-18 or LNPs having a substantially similar formulation of CPT163-21-18, that includes: (a) about 0.1 mol % to about 10 mol % (e.g., about 1.8 mol %) of the compound of the copolymer described herein (e.g., L81); (b) about 10 mol % to about 30 mol % (e.g., about 19 mol %) of ALC-0315 or a derivative thereof; (c) about 10 mol % to about 30 mol % (e.g., about 21 mol %) of DSPC or a derivative thereof, and about 10 mol % to about 30 mol % (e.g., about 21 mol %) of DOPE or a derivative thereof; (d) about 20 mol % to about 50 mol % (e.g., about 36 mol %) of cholesterol or a derivative thereof; and (e) about 0.5 mol % to about 5 mol % (e.g., about 1.4 mol %) of PEG2000-DSPE or a derivative thereof.

In some embodiments, provided herein is CPT147E or LNPs having a substantially similar formulation of CPT147E, that includes: (a) about 0.1 mol % to about 10 mol % (e.g., about 6.8 mol %) of the compound of the copolymer described herein (e.g., L121); (b) about mol % to about 30 mol % (e.g., about 18 mol %) of ALC-0315 or a derivative thereof; (c) about 10 mol % to about 30 mol % (e.g., about 19.8 mol %) of DSPC or a derivative thereof, and about 10 mol % to about 30 mol % (e.g., about 19.8 mol %) of DOPE or a derivative thereof; (d) about 20 mol % to about 50 mol % (e.g., about 34.2 mol %) of cholesterol or a derivative thereof; and (e) about 0.5 mol % to about 5 mol % (e.g., about 1.4 mol %) of PEG2000-DSPE or a derivative thereof.

In some embodiments, provided herein is CPT147E-05 or LNPs having a substantially similar formulation of CPT147E-05, that includes: (a) about 0.1 mol % to about mol % (e.g., about 6.8 mol %) of the compound of the copolymer described herein (e.g., L121); (b) about 10 mol % to about 30 mol % (e.g., about 18 mol %) of ALC-0315 or a derivative thereof; (c) about 10 mol % to about 30 mol % (e.g., about 19.8 mol %) of DSPC or a derivative thereof, and about 10 mol % to about 30 mol % (e.g., about 19.8 mol %) of DOPE or a derivative thereof; (d) about 20 mol % to about 50 mol % (e.g., about 34.2 mol %) of cholesterol or a derivative thereof; and (e) about 0.5 mol % to about 5 mol % (e.g., about 1.4 mol %) of PEG2000-DSPE or a derivative thereof.

In some embodiments, provided herein is CPT149E-01 or LNPs having a substantially similar formulation of CPT149E-01, that includes: (a) about 0.1 mol % to about mol % (e.g., about 6.8 mol %) of the compound of the copolymer described herein (e.g., L121); (b) about 10 mol % to about 30 mol % (e.g., about 18 mol %) of SM-102 or a derivative thereof; (c) about 10 mol % to about 30 mol % (e.g., about 19.8 mol %) of DSPC or a derivative thereof, and about 10 mol % to about 30 mol % (e.g., about 19.8 mol %) of DOPE or a derivative thereof; (d) about 20 mol % to about 50 mol % (e.g., about 34.2 mol %) of cholesterol or a derivative thereof; and (e) about 0.5 mol % to about 5 mol % (e.g., about 1.5 mol %) of PEG2000-DSPE or a derivative thereof.

In some embodiments, provided herein is CPT162E-01 or LNPs having a substantially similar formulation of CPT162E-01, that includes: (a) about 0.1 mol % to about mol % (e.g., about 6.8 mol %) of the compound of the copolymer described herein (e.g., L121); (b) about 10 mol % to about 30 mol % (e.g., about 18 mol %) of DLin-KC2-DMA or a derivative thereof; (c) about 10 mol % to about 30 mol % (e.g., about 19.8 mol %) of DSPC or a derivative thereof, and about 10 mol % to about 30 mol % (e.g., about 19.8 mol %) of DOPE or a derivative thereof; (d) about 20 mol % to about 50 mol % (e.g., about 34.2 mol %) of cholesterol or a derivative thereof; and (e) about 0.5 mol % to about 5 mol % (e.g., about 1.5 mol %) of PEG2000-DSPE or a derivative thereof.

In some embodiments, provided herein is CPT147E-04 or LNPs having a substantially similar formulation of CPT147E-04, that includes: (a) about 0.1 mol % to about mol % (e.g., about 5 mol %) of the compound of the copolymer described herein (e.g., L121); (b) about 1 mol % to about 20 mol % (e.g., about 10 mol %) of ALC-0315 or a derivative thereof; (c) about 10 mol % to about 30 mol % (e.g., about 22.5 mol %) of DSPC or a derivative thereof, and about 10 mol % to about 30 mol % (e.g., about 22.5 mol %) of DOPE or a derivative thereof; (d) about 20 mol % to about 50 mol % (e.g., about 38.5 mol %) of cholesterol or a derivative thereof; and (e) about 0.5 mol % to about 5 mol % (e.g., about 1.5 mol %) of PEG2000-DSPE or a derivative thereof.

In some embodiments, provided herein is CPT163-10 or LNPs having a substantially similar formulation of CPT163-10, that includes: (a) about 0.1 mol % to about 10 mol % (e.g., about 1.9 mol %) of the compound of the copolymer described herein (e.g., L81); (b) about 1 mol % to about 30 mol % (e.g., about 19 mol %) of ALC-0315 or a derivative thereof; (c) about 10 mol % to about 30 mol % (e.g., about 20.9 mol %) of DSPC or a derivative thereof, and about 10 mol % to about 30 mol % (e.g., about 20.9 mol %) of DOPE or a derivative thereof; (d) about 20 mol % to about 50 mol % (e.g., about 36 mol %) of cholesterol or a derivative thereof; and (e) about 0.5 mol % to about 5 mol % (e.g., about 1.4 mol %) of PEG2000-DSPE or a derivative thereof.

In some embodiments, provided herein is CPT161E or LNPs having a substantially similar formulation of CPT161E, that includes: (a) about 0.1 mol % to about 10 mol % (e.g., about 6.8 mol %) of the compound of the copolymer described herein (e.g., L121); (b) about 1 mol % to about 30 mol % (e.g., about 18 mol %) of DLin-DMA or a derivative thereof; (c) about 10 mol % to about 30 mol % (e.g., about 19.8 mol %) of DSPC or a derivative thereof, and about 10 mol % to about 30 mol % (e.g., about 19.8 mol %) of DOPE or a derivative thereof; (d) about 20 mol % to about 50 mol % (e.g., about 34.2 mol %) of cholesterol or a derivative thereof; and (e) about 0.5 mol % to about 5 mol % (e.g., about 1.5 mol %) of PEG2000-DSPE or a derivative thereof.

In some embodiments, provided herein is CPT164E or LNPs having a substantially similar formulation of CPT164E, that includes: (a) about 0.1 mol % to about 10 mol % (e.g., about 4.6 mol %) of the compound of the copolymer described herein (e.g., L92); (b) about 1 mol % to about 30 mol % (e.g., about 18.3 mol %) of DLin-KC2-DMA or a derivative thereof; (c) about 10 mol % to about 30 mol % (e.g., about 20.3 mol %) of DSPC or a derivative thereof, and about 10 mol % to about 30 mol % (e.g., about 20.3 mol %) of DOPE or a derivative thereof; (d) about 20 mol % to about 50 mol % (e.g., about 35.1 mol %) of cholesterol or a derivative thereof; and (e) about 0.5 mol % to about 5 mol % (e.g., about 1.4 mol %) of PEG2000-DSPE or a derivative thereof.

In some embodiments, provided herein is CPT149-01 or LNPs having a substantially similar formulation of CPT149-01, that includes: (a) about 0.1 mol % to about 10 mol % (e.g., about 6.8 mol %) of the compound of the copolymer described herein (e.g., L121); (b) about 1 mol % to about 30 mol % (e.g., about 18 mol %) of SM-102 or a derivative thereof; (c) about 10 mol % to about 30 mol % (e.g., about 19.8 mol %) of DSPC or a derivative thereof, and about 10 mol % to about 30 mol % (e.g., about 19.8 mol %) of DOPE or a derivative thereof; (d) about 20 mol % to about 50 mol % (e.g., about 34.2 mol %) of cholesterol or a derivative thereof; and (e) about 0.5 mol % to about 5 mol % (e.g., about 1.5 mol %) of PEG2000-DSPE or a derivative thereof.

In some embodiments, provided herein is CPT200-07 or LNPs having a substantially similar formulation of CPT200-07, that includes: (a) about 0.1 mol % to about 10 mol % (e.g., about 2.2 mol %) of the compound of the copolymer described herein (e.g., L81); (b) about 1 mol % to about 30 mol % (e.g., about 22.3 mol %) of SM-102 or a derivative thereof; (c) about mol % to about 30 mol % (e.g., about 15.6 mol %) of DSPC or a derivative thereof, and about 10 mol % to about 30 mol % (e.g., about 15.6 mol %) of DOPE or a derivative thereof; (d) about 30 mol % to about 60 mol % (e.g., about 43 mol %) of cholesterol or a derivative thereof; and (e) about 0.5 mol % to about 5 mol % (e.g., about 1.1 mol %) of PEG2000-DSPE or a derivative thereof.

In some embodiments, provided herein is CPT153E or LNPs having a substantially similar formulation of CPT153E, that includes: (a) about 0.1 mol % to about 10 mol % (e.g., about 6.8 mol %) of the compound of the copolymer described herein (e.g., L121); (b) about 1 mol % to about 30 mol % (e.g., about 17.9 mol %) of DOTAP or a derivative thereof; (c) about mol % to about 30 mol % (e.g., about 19.9 mol %) of DSPC or a derivative thereof, and about 10 mol % to about 30 mol % (e.g., about 19.9 mol %) of DOPE or a derivative thereof; (d) about 30 mol % to about 60 mol % (e.g., about 34.1 mol %) of cholesterol or a derivative thereof; and (e) about 0.5 mol % to about 5 mol % (e.g., about 1.4 mol %) of PEG2000-DSPE or a derivative thereof.

In some embodiments, provided herein is CPT189 or LNPs having a substantially similar formulation of CPT189, that includes: (a) about 0.1 mol % to about 10 mol % (e.g., about 2.4 mol %) of the compound of the copolymer described herein (e.g., L31R₁); (b) about 1 mol % to about 30 mol % (e.g., about 21.7 mol %) of ALC-0315 or a derivative thereof; (c) about 10 mol % to about 30 mol % (e.g., about 16.9 mol %) of DSPC or a derivative thereof, and about 10 mol % to about 30 mol % (e.g., about 16.9 mol %) of DOPE or a derivative thereof; (d) about 30 mol % to about 60 mol % (e.g., about 41 mol %) of cholesterol or a derivative thereof; and (e) about 0.5 mol % to about 5 mol % (e.g., about 1.2 mol %) of PEG2000-DSPE or a derivative thereof.

In some embodiments, provided herein is CPT202 or LNPs having a substantially similar formulation of CPT202, that includes: (a) about 0.1 mol % to about 10 mol % (e.g., about 2.4 mol %) of the compound of the copolymer described herein (e.g., L17R₄); (b) about 1 mol % to about 30 mol % (e.g., about 21.7 mol %) of ALC-0315 or a derivative thereof; (c) about 10 mol % to about 30 mol % (e.g., about 16.9 mol %) of DSPC or a derivative thereof, and about 10 mol % to about 30 mol % (e.g., about 16.9 mol %) of DOPE or a derivative thereof; (d) about 30 mol % to about 60 mol % (e.g., about 41 mol %) of cholesterol or a derivative thereof; and (e) about 0.5 mol % to about 5 mol % (e.g., about 1.2 mol %) of PEG2000-DSPE or a derivative thereof.

In some embodiments, provided herein is CPT147P or LNPs having a substantially similar formulation of CPT147P, that includes: (a) about 0.1 mol % to about 10 mol % (e.g., about 5 mol %) of the compound of the copolymer described herein (e.g., L121); (b) about 1 mol % to about 30 mol % (e.g., about 18.1 mol %) of ALC-0315 or a derivative thereof; (c) about 10 mol % to about 30 mol % (e.g., about 18.1 mol %) of DSPC or a derivative thereof, and about 10 mol % to about 30 mol % (e.g., about 18.1 mol %) of DOPE or a derivative thereof; (d) about 30 mol % to about 60 mol % (e.g., about 39.2 mol %) of cholesterol or a derivative thereof; and (e) about 0.5 mol % to about 5 mol % (e.g., about 1.5 mol %) of PEG2000-DSPE or a derivative thereof.

In some embodiments, provided herein is CPT149E or LNPs having a substantially similar formulation of CPT149E, that includes: (a) about 0.1 mol % to about 10 mol % (e.g., about 6.8 mol %) of the compound of the copolymer described herein (e.g., L121); (b) about 1 mol % to about 30 mol % (e.g., about 18 mol %) of SM-102 or a derivative thereof; (c) about 10 mol % to about 30 mol % (e.g., about 19.8 mol %) of DSPC or a derivative thereof, and about 10 mol % to about 30 mol % (e.g., about 19.8 mol %) of DOPE or a derivative thereof; (d) about 30 mol % to about 60 mol % (e.g., about 34.1 mol %) of cholesterol or a derivative thereof; and (e) about 0.5 mol % to about 5 mol % (e.g., about 1.5 mol %) of PEG2000-DSPE or a derivative thereof.

In some embodiments, provided herein is CPT201 or LNPs having a substantially similar formulation of CPT201, that includes: (a) about 0.1 mol % to about 10 mol % (e.g., about 2.2 mol %) of the compound of the copolymer described herein (e.g., PPO₂₇₀₀); (b) about 1 mol % to about 30 mol % (e.g., about 22.5 mol %) of ALC-0315 or a derivative thereof; (c) about 10 mol % to about 30 mol % (e.g., about 15.7 mol %) of DSPC or a derivative thereof, and about 10 mol % to about 30 mol % (e.g., about 15.7 mol %) of DOPE or a derivative thereof; (d) about 30 mol % to about 60 mol % (e.g., about 42.7 mol %) of cholesterol or a derivative thereof; and (e) about 0.5 mol % to about 5 mol % (e.g., about 1.1 mol %) of PEG2000-DSPE or a derivative thereof.

### Physical Properties

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, such as particle size, polydispersity index, and zeta potential.

In some embodiments, the LNPs or LNPs having the nanoparticle composition described herein have a N/P ratio of about 0.1 to about 10, e.g., about 0.1 to about 9, about 0.1 to about 8, about 0.1 to about 7, about 0.1 to about 6, about 0.1 to about 5, about 0.1 to about 4, about 0.1 to about 3, about 0.1 to about 2, about 0.1 to about 1, about 0.1 to about 0.9, about 0.1 to about 0.8, about 0.1 to about 0.7, about 0.1 to about 0.6, about 0.1 to about 0.5, about 0.1 to about 0.4, about 0.1 to about 0.3, about 0.1 to about 0.2, about 0.2 to about 10, about 0.2 to about 9, about 0.2 to about 8, about 0.2 to about 7, about 0.2 to about 6, about 0.2 to about 5, about 0.2 to about 4, about 0.2 to about 3, about 0.2 to about 2, about 0.2 to about 1, about 0.2 to about 0.9, about 0.2 to about 0.8, about 0.2 to about 0.7, about 0.2 to about 0.6, about 0.2 to about 0.5, about 0.2 to about 0.4, about 0.2 to about 0.3, about 0.3 to about 10, about 0.3 to about 9, about 0.3 to about 8, about 0.3 to about 7, about 0.3 to about 6, about 0.3 to about 5, about 0.3 to about 4, about 0.3 to about 3, about 0.3 to about 2, about 0.3 to about 1, about 0.3 to about 0.9, about 0.3 to about 0.8, about 0.3 to about 0.7, about 0.3 to about 0.6, about 0.3 to about 0.5, about 0.3 to about 0.4, about 0.4 to about 10, about 0.4 to about 9, about 0.4 to about 8, about 0.4 to about 7, about 0.4 to about 6, about 0.4 to about 5, about 0.4 to about 4, about 0.4 to about 3, about 0.4 to about 2, about 0.4 to about 1, about 0.4 to about 0.9, about 0.4 to about 0.8, about 0.4 to about 0.7, about 0.4 to about 0.6, about 0.4 to about 0.5, about 0.5 to about 10, about 0.5 to about 9, about 0.5 to about 8, about 0.5 to about 7, about 0.5 to about 6, about 0.5 to about 5, about 0.5 to about 4, about 0.5 to about 3, about 0.5 to about 2, about 0.5 to about 1, about 0.5 to about 0.9, about 0.5 to about 0.8, about 0.5 to about 0.7, about 0.5 to about 0.6, about 0.6 to about 10, about 0.6 to about 9, about 0.6 to about 8, about 0.6 to about 7, about 0.6 to about 6, about 0.6 to about 5, about 0.6 to about 4, about 0.6 to about 3, about 0.6 to about 2, about 0.6 to about 1, about 0.6 to about 0.9, about 0.6 to about 0.8, about 0.6 to about 0.7, about 0.7 to about 10, about 0.7 to about 9, about 0.7 to about 8, about 0.7 to about 7, about 0.7 to about 6, about 0.7 to about 5, about 0.7 to about 4, about 0.7 to about 3, about 0.7 to about 2, about 0.7 to about 1, about 0.7 to about 0.9, about 0.7 to about 0.8, about 0.8 to about 10, about 0.8 to about 9, about 0.8 to about 8, about 0.8 to about 7, about 0.8 to about 6, about 0.8 to about 5, about 0.8 to about 4, about 0.8 to about 3, about 0.8 to about 2, about 0.8 to about 1, about 0.8 to about 0.9, about 0.9 to about 10, about 0.9 to about 9, about 0.9 to about 8, about 0.9 to about 7, about 0.9 to about 6, about 0.9 to about 5, about 0.9 to about 4, about 0.9 to about 3, about 0.9 to about 2, about 0.9 to about 1, about 1 to about 10, about 1 to about 9, about 1 to about 8, about 1 to about 7, about 1 to about 6, about 1 to about 5, about 1 to about 4, about 1 to about 3, about 1 to about 2, about 2 to about 10, about 2 to about 9, about 2 to about 8, about 2 to about 7, about 2 to about 6, about 2 to about 5, about 2 to about 4, about 2 to about 3, about 3 to about 10, about 3 to about 9, about 3 to about 8, about 3 to about 7, about 3 to about 6, about 3 to about 5, about 3 to about 4, about 4 to about 10, about 4 to about 9, about 4 to about 8, about 4 to about 7, about 4 to about 6, about 4 to about 5, about 5 to about 10, about 5 to about 9, about to about 8, about 5 to about 7, about 5 to about 6, about 6 to about 10, about 6 to about 9, about 6 to about 8, about 6 to about 7, about 7 to about 10, about 7 to about 9, about 7 to about 8, about 8 to about 10, about 8 to about 9, or about 9 to about 10.

In some embodiments, the N/P ratio of the LNPs or LNPs having the nanoparticle compositions described herein is about 0.25 to about 1, e.g., about 0.26, about 0.27, about 0.28, about 0.29, about 0.3, about 0.31, about 0.32, about 0.33, about 0.34, about 0.35, about 0.36, about 0.37, about 0.38, about 0.39, about 0.4, about 0.41, about 0.42, about 0.43, about 0.44, about 0.45, about 0.46, about 0.47, about 0.48, about 0.49, about 0.5, about 0.51, about 0.52, about 0.53, about 0.54, about 0.55, about 0.56, about 0.57, about 0.58, about 0.59, about 0.6, about 0.61, about 0.62, about 0.63, about 0.64, about 0.65, about 0.66, about 0.67, about 0.68, about 0.69, about 0.7, about 0.71, about 0.72, about 0.73, about 0.74, about 0.75, about 0.76, about 0.77, about 0.78, about 0.79, about 0.8, about 0.81, about 0.82, about 0.83, about 0.84, about 0.85, about 0.86, about 0.87, about 0.88, about 0.89, about 0.9, about 0.91, about 0.92, about 0.93, about 0.94, about 0.95, about 0.96, about 0.97, about 0.98, about 0.99, about 1, or any ranges between two of the above values.

As used herein, the “N/P ratio” is the molar ratio of the ionizable nitrogen atoms in an ionizable lipid or the permanently charged nitrogen atoms in a permanently charged cationic lipid relative to the phosphate groups in the backbone of a nucleic acid. In some embodiments, the nucleic acid is a DNA or an RNA (e.g., an mRNA).

In some embodiments, the LNPs or LNPs having the nanoparticle composition described herein is stable after storage at a temperature of about 20° C. to about 40° C. (e.g., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.) for at least 1 day, 2 days, 3 days, 4 days, 5 days, 6 days, 1 week, 2 weeks, or 3 weeks. In some embodiments, the stability of the LNPs is determined by detecting the transfection efficiency of the LNPs after the storage.

In some embodiments, the LNPs or LNPs having the nanoparticle composition described herein have a hydrophilic lipophilic balance (HLB) value from about 1 to about 18, e.g., 1, 2, 3, 4, 5, 6, 7, 8, 9, 10, 11, 12, 13, 14, 15, 16, 17, or 18. HLB defines the affinity of a surfactant toward a solvent and ranges from 0 to 20. The greater the HLB, the greater the affinity of the surfactant to water, while in contrast the lower the HLB, the higher the oil affinity of the surfactant. Details of HLB determination can be found, e.g., in Griffin, W. C. “Calculation of HLB values of non-ionic surfactants.” J. Soc. Cosmet. Chem. 5 (1954): 249-256, which is incorporated herein by reference in its entirety. For example, the HLB value is about 1 for L121; about 2 for L81, and about 6 for L92.

In some embodiments, the LNPs or LNPs having the nanoparticle composition described herein have a mean size of about 30 nm to about 2000 nm, e.g., about 30 nm to about 1800 nm, about 30 nm to about 1600 nm, about 30 nm to about 1400 nm, about 30 nm to about 1200 nm, about 30 nm to about 1000 nm, about 30 nm to about 900 nm, about 30 nm to about 800 nm, about 30 nm to about 700 nm, about 30 nm to about 600 nm, about 30 nm to about 500 nm, about 30 nm to about 400 nm, about 30 nm to about 300 nm, about 30 nm to about 200 nm, about 30 nm to about 100 nm, about 30 nm to about 90 nm, about 30 nm to about 80 nm, about 30 nm to about 70 nm, about 30 nm to about 60 nm, about 30 nm to about 50 nm, about 30 nm to about 40 nm, about 40 nm to about 2000 nm, about 40 nm to about 1800 nm, about 40 nm to about 1600 nm, about 40 nm to about 1400 nm, about 40 nm to about 1200 nm, about 40 nm to about 1000 nm, about 40 nm to about 900 nm, about 40 nm to about 800 nm, about 40 nm to about 700 nm, about 40 nm to about 600 nm, about 40 nm to about 500 nm, about 40 nm to about 400 nm, about 40 nm to about 300 nm, about 40 nm to about 200 nm, about 40 nm to about 100 nm, about 40 nm to about 90 nm, about 40 nm to about 80 nm, about 40 nm to about 70 nm, about 40 nm to about 60 nm, about 40 nm to about 50 nm, about 50 nm to about 2000 nm, about 50 nm to about 1800 nm, about 50 nm to about 1600 nm, about 50 nm to about 1400 nm, about 50 nm to about 1200 nm, about 50 nm to about 1000 nm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 200 nm, about 50 nm to about 100 nm, about 50 nm to about 90 nm, about 50 nm to about 80 nm, about 50 nm to about 70 nm, about 50 nm to about 60 nm, about 60 nm to about 2000 nm, about 60 nm to about 1800 nm, about 60 nm to about 1600 nm, about 60 nm to about 1400 nm, about 60 nm to about 1200 nm, about 60 nm to about 1000 nm, about 60 nm to about 900 nm, about 60 nm to about 800 nm, about 60 nm to about 700 nm, about 60 nm to about 600 nm, about 60 nm to about 500 nm, about 60 nm to about 400 nm, about 60 nm to about 300 nm, about 60 nm to about 200 nm, about 60 nm to about 100 nm, about 60 nm to about 90 nm, about 60 nm to about 80 nm, about 60 nm to about 70 nm, about 70 nm to about 2000 nm, about 70 nm to about 1800 nm, about 70 nm to about 1600 nm, about 70 nm to about 1400 nm, about 70 nm to about 1200 nm, about 70 nm to about 1000 nm, about 70 nm to about 900 nm, about 70 nm to about 800 nm, about 70 nm to about 700 nm, about 70 nm to about 600 nm, about 70 nm to about 500 nm, about 70 nm to about 400 nm, about 70 nm to about 300 nm, about 70 nm to about 200 nm, about 70 nm to about 100 nm, about 70 nm to about 90 nm, about 70 nm to about 80 nm, about 80 nm to about 2000 nm, about 80 nm to about 1800 nm, about 80 nm to about 1600 nm, about 80 nm to about 1400 nm, about 80 nm to about 1200 nm, about 80 nm to about 1000 nm, about 80 nm to about 900 nm, about 80 nm to about 800 nm, about 80 nm to about 700 nm, about 80 nm to about 600 nm, about 80 nm to about 500 nm, about 80 nm to about 400 nm, about 80 nm to about 300 nm, about 80 nm to about 200 nm, about 80 nm to about 100 nm, about 80 nm to about 90 nm, about 90 nm to about 2000 nm, about 90 nm to about 1800 nm, about 90 nm to about 1600 nm, about 90 nm to about 1400 nm, about 90 nm to about 1200 nm, about 90 nm to about 1000 nm, about 90 nm to about 900 nm, about 90 nm to about 800 nm, about 90 nm to about 700 nm, about 90 nm to about 600 nm, about 90 nm to about 500 nm, about 90 nm to about 400 nm, about 90 nm to about 300 nm, about 90 nm to about 200 nm, about 90 nm to about 100 nm, about 100 nm to about 2000 nm, about 100 nm to about 1800 nm, about 100 nm to about 1600 nm, about 100 nm to about 1400 nm, about 100 nm to about 1200 nm, about 100 nm to about 1000 nm, about 100 nm to about 900 nm, about 100 nm to about 800 nm, about 100 nm to about 700 nm, about 100 nm to about 600 nm, about 100 nm to about 500 nm, about 100 nm to about 400 nm, about 100 nm to about 300 nm, about 100 nm to about 200 nm, about 200 nm to about 2000 nm, about 200 nm to about 1800 nm, about 200 nm to about 1600 nm, about 200 nm to about 1400 nm, about 200 nm to about 1200 nm, about 200 nm to about 1000 nm, about 200 nm to about 900 nm, about 200 nm to about 800 nm, about 200 nm to about 700 nm, about 200 nm to about 600 nm, about 200 nm to about 500 nm, about 200 nm to about 400 nm, about 200 nm to about 300 nm, about 300 nm to about 2000 nm, about 300 nm to about 1800 nm, about 300 nm to about 1600 nm, about 300 nm to about 1400 nm, about 300 nm to about 1200 nm, about 300 nm to about 1000 nm, about 300 nm to about 900 nm, about 300 nm to about 800 nm, about 300 nm to about 700 nm, about 300 nm to about 600 nm, about 300 nm to about 500 nm, about 300 nm to about 400 nm, about 400 nm to about 2000 nm, about 400 nm to about 1800 nm, about 400 nm to about 1600 nm, about 400 nm to about 1400 nm, about 400 nm to about 1200 nm, about 400 nm to about 1000 nm, about 400 nm to about 900 nm, about 400 nm to about 800 nm, about 400 nm to about 700 nm, about 400 nm to about 600 nm, about 400 nm to about 500 nm, about 500 nm to about 2000 nm, about 500 nm to about 1800 nm, about 500 nm to about 1600 nm, about 500 nm to about 1400 nm, about 500 nm to about 1200 nm, about 500 nm to about 1000 nm, about 500 nm to about 900 nm, about 500 nm to about 800 nm, about 500 nm to about 700 nm, about 500 nm to about 600 nm, about 600 nm to about 2000 nm, about 600 nm to about 1800 nm, about 600 nm to about 1600 nm, about 600 nm to about 1400 nm, about 600 nm to about 1200 nm, about 600 nm to about 1000 nm, about 600 nm to about 900 nm, about 600 nm to about 800 nm, about 600 nm to about 700 nm, about 700 nm to about 2000 nm, about 700 nm to about 1800 nm, about 700 nm to about 1600 nm, about 700 nm to about 1400 nm, about 700 nm to about 1200 nm, about 700 nm to about 1000 nm, about 700 nm to about 900 nm, about 700 nm to about 800 nm, about 800 nm to about 2000 nm, about 800 nm to about 1800 nm, about 800 nm to about 1600 nm, about 800 nm to about 1400 nm, about 800 nm to about 1200 nm, about 800 nm to about 1000 nm, about 800 nm to about 900 nm, about 900 nm to about 2000 nm, about 900 nm to about 1800 nm, about 900 nm to about 1600 nm, about 900 nm to about 1400 nm, about 900 nm to about 1200 nm, about 900 nm to about 1000 nm, about 1000 nm to about 2000 nm, about 1000 nm to about 1800 nm, about 1000 nm to about 1600 nm, about 1000 nm to about 1400 nm, about 1000 nm to about 1200 nm, about 1200 nm to about 2000 nm, about 1200 nm to about 1800 nm, about 1200 nm to about 1600 nm, about 1200 nm to about 1400 nm, about 1400 nm to about 2000 nm, about 1400 nm to about 1800 nm, about 1400 nm to about 1600 nm, about 1600 nm to about 2000 nm, about 1600 nm to about 1800 nm, or about 1800 nm to about 2000 nm. In some embodiments, the mean size of the LNPs is mass-averaged size, volume-averaged size, intensity-averaged size, or number-averaged size. In some embodiments, the mean size of the LNPs is determined by light scattering-based methods.

The LNPs or LNPs having the nanoparticle composition described herein may be relatively homogenous. A polydispersity index may be used to indicate the homogeneity of a nanoparticle composition, e.g., the particle size distribution of the nanoparticle compositions. A small (e.g., less than 0.5) polydispersity index generally indicates a narrow particle size distribution. In some embodiments, the LNPs or LNPs having the nanoparticle composition described herein have a polydispersity index that is about 0.001 to about 0.5, about 0.001 to about 0.4, about 0.001 to about 0.3, about 0.001 to about 0.2, about 0.001 to about 0.1, about 0.001 to about 0.05, about 0.001 to about 0.01, about 0.001 to about 0.005, about 0.005 to about 0.5, about 0.005 to about 0.4, about 0.005 to about 0.3, about 0.005 to about 0.2, about 0.005 to about 0.1, about 0.005 to about 0.05, about 0.005 to about 0.01, about 0.01 to about 0.5, about 0.01 to about 0.4, about 0.01 to about 0.3, about 0.01 to about 0.2, about 0.01 to about 0.1, about 0.01 to about 0.05, about 0.05 to about 0.5, about 0.05 to about 0.4, about 0.05 to about 0.3, about 0.05 to about 0.2, about 0.05 to about 0.1, about 0.1 to about 0.5, about 0.1 to about 0.4, about 0.1 to about 0.3, about 0.1 to about 0.2, about 0.2 to about 0.5, about 0.2 to about 0.4, about 0.2 to about 0.3, about 0.3 to about 0.5, about 0.3 to about 0.4, or about 0.4 to about 0.5.

The zeta potential of a LNP may be used to indicate the electrokinetic potential of the composition. For example, the zeta potential may describe the surface charge of a LNP. LNPs with relatively low charges, positive or negative, are generally desirable, as more highly charged species may interact undesirably with cells, tissues, and other elements in the body.

In some embodiments, the LNPs or LNPs having the nanoparticle composition described herein have a zeta potential of about −30 mV to about +20 mV, about −30 mV to about +10 mV, about −30 mV to about 0 mV, about −30 mV to about −10 mV, about −30 mV to about −20 mV, −20 mV to about +20 mV, about −20 mV to about +10 mV, about −20 mV to about 0 mV, about −20 mV to about −10 mV, −10 mV to about +20 mV, about −10 mV to about +10 mV, about −10 mV to about 0 mV, about 0 mV to about +20 mV, about 0 mV to about +10 mV, or about 10 mV to about 20 mV.

In some embodiments, the w/w ratio of the lipid component to the cargo molecule (e.g., any of the nucleic acids described herein) is about 2:1 to about 50:1, about 2:1 to about 40:1, about 2:1 to about 30:1, about 2:1 to about 20:1, about 2:1 to about 10:1, about 2:1 to about 8:1, about 2:1 to about 6:1, about 2:1 to about 4:1, about 4:1 to about 50:1, about 4:1 to about 40:1, about 4:1 to about 30:1, about 4:1 to about 20:1, about 4:1 to about 10:1, about 4:1 to about 8:1, about 4:1 to about 6:1, about 6:1 to about 50:1, about 6:1 to about 40:1, about 6:1 to about 30:1, about 6:1 to about 20:1, about 6:1 to about 10:1, about 6:1 to about 8:1, about 8:1 to about 50:1, about 8:1 to about 40:1, about 8:1 to about 30:1, about 8:1 to about 20:1, about 8:1 to about 10:1, about 10:1 to about 50:1, about 10:1 to about 40:1, about 10:1 to about 30:1, about 10:1 to about 20:1, about 20:1 to about 50:1, about 20:1 to about 40:1, about 20:1 to about 30:1, about 30:1 to about 50:1, about 30:1 to about 40:1, or about 40:1 to about 50:1. The amount of the cargo molecules in a nanoparticle composition may, for example, be measured using absorption spectroscopy (e.g., ultraviolet-visible spectroscopy).

In some embodiments, the LNPs or LNPs having the nanoparticle composition described herein encapsulate a cargo molecule (e.g., any of the nucleic acids described herein). In some embodiments, the encapsulation efficiency is at least 50% a, at least 55%, at least 60%, at least 65%, at least 70%, at least 75%, at least 80%, at least 85%, or at least 90%.

### Pharmaceutical Compositions

In some embodiments, the LNPs or LNPs having the nanoparticle composition described herein is associated with a therapeutic medicine, a vaccine, gene editing, or cell-based therapies.

The nanoparticle compositions disclosed herein may be formulated in whole or in part as pharmaceutical compositions. Pharmaceutical compositions of the disclosure may include one or more nanoparticle compositions. For example, a pharmaceutical composition may include one or more nanoparticle compositions including one or more different cargo molecules (e.g., DNAs or mRNAs). Pharmaceutical compositions of the disclosure may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, M D, 2006. Conventional excipients and accessory ingredients may be used in any pharmaceutical composition of the disclosure, except insofar as any conventional excipient or accessory ingredient may be incompatible with one or more components of a nanoparticle composition of the disclosure. An excipient or accessory ingredient may be incompatible with a component of a nanoparticle composition if its combination with the component may result in any undesirable biological effect or otherwise deleterious effect.

In some embodiments, one or more excipients or accessory ingredients may make up greater than 50% of the total mass or volume of a pharmaceutical composition including a nanoparticle composition of the disclosure. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more nanoparticle compositions, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more nanoparticle compositions.

Nanoparticle compositions and/or pharmaceutical compositions including one or more nanoparticle compositions may be administered to any patient or subject, including those patients or subjects that may benefit from a therapeutic effect provided by the delivery of a DNA or an mRNA to one or more particular cells, tissues, organs, or systems or groups thereof, such as the renal system. Although the descriptions provided herein of nanoparticle compositions and pharmaceutical compositions including nanoparticle compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation.

Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more nanoparticle compositions may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage.

Pharmaceutical compositions disclosed herein may be prepared in a variety of forms suitable for a variety of routes and methods of administration. For example, pharmaceutical compositions of the disclosure may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S. P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient. Solid dosage forms for oral administration include capsules, tablets, pills, films, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay, silicates), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner.

Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants, and/or patches.

Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (wt/wt) active ingredient, although the concentration of active ingredient may be as high as the solubility limit of the active ingredient in the solvent.

Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e., by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

### Method of Making LNPs

The present disclosure provides methods of making the LNPs described herein (e.g., any of the PALNPs described herein), or LNPs having the nanoparticle compositions described herein (e.g., any of the nanoparticle compositions described herein). The LNPs can be generated using the nPort™ technology disclosed in U.S. Pat. No. 11,497,715B2 and U.S. Pat. No. 9,693,958B2, each of which is incorporated herein by reference in its entirety.

In one aspect, the disclosure is related to methods of making the LNPs (e.g., any of the PALNPs described herein, or LNPs having the nanoparticle compositions described herein (e.g., any of the nanoparticle compositions described herein)), comprising: (a) introducing one or more streams of a lipid solution in a water-miscible organic solvent via a first set of one or more inlet ports connected to a mixing chamber, wherein the lipid solution comprises the lipid component and the polymer component, (b) introducing one or more streams of an aqueous solution via a second set of one or more inlet ports connected to the mixing chamber, (c) mixing the one or more streams of the lipid solution and the one or more streams of the aqueous solution in a mixing chamber to generated the LNP, and (d) recovering the LNP via one or more outlet ports connected to the mixing chamber.

In some embodiments, the aqueous solution does not include a nucleic acid. For example, after generation of the blank LNP, they can be loaded with a nucleic acid (e.g., any of the nucleic acids described herein), e.g., by mixing the blank LNP with a solution containing the nucleic acid at about 30-40° C. (e.g., about 37° C.) for about 1-30 minutes (e.g., about 10 minutes). In some embodiments, the volume ratio of the blank LNP to the nucleic acid is about 1:1, about 2:1, about 3:1, about 4:1, about 5:1, about 6:1, about 7:1, or about 8:1. In some embodiments, the relative amount (e.g., volume ratio) of the blank LNP to the nucleic acid determines the N/P ratio of the loaded LNP as described herein.

In some embodiments, the aqueous solution includes a nucleic acid (e.g., any of the nucleic acids described herein). For example, after mixing the one or more streams of the lipid solution and the one or more streams of the aqueous solution in the mixing chamber, loaded LNP containing the nucleic acid are instantaneously formed. Thus, there is no need to perform an additional loading step to generate loaded LNP containing the nucleic acid.

The disclosure provides a device adapted to perform the methods disclosed herein, such as a manifold system described herein. In some embodiments, the present technology provides a device for preparing LNP encapsulating an API that includes a manifold that may have a mixing chamber, at least one lipid solution inlet port connected to the chamber; and a plurality of aqueous solution inlet ports connected to the chamber. In a preferred embodiment, the device may include a LNP solution outlet port connected to the chamber.

Preferably, the device may include a reservoir for a lipid solution which is connected to the lipid solution inlet port by a lipid solution conduit, and a reservoir for an aqueous solution which is connected to the aqueous solution inlet ports by an aqueous solution conduit.

The inlet ports for the lipid and aqueous solutions, and the exit port for the liposome solutions may have an internal diameter which is the same or different. Preferably inlet and outlet ports have an internal diameter from about 0.1 mm to about 10 mm. More preferably the ports have an internal diameter from about 0.15 mm to about 5 mm.

In some embodiments, the mixing chamber is located at the point of conversion of the conduits and may itself be formed by two or more conduits passing through each other, or intersecting, without any change in the shape of the conduits. For instance, the mixing chamber can formed by drilling in a solid material two or more pass-through channels all intersecting at the point of conversion. In addition, one or more conduits may be sealed so that there is no passage of fluid permitted through the conduit. Such seal may be located either immediately prior to the point of intersection or distantly therefrom.

In some embodiments, one manifold may contain more than one mixing chamber. For example, one set of inlet ports intersect at one chamber, and another group of inlet ports intersect at another chamber, and the two chambers are connected by conduits to the third mixing chamber that is connected to an outlet port.

Preferably, a pump is used to induce a positive flow to the lipid solution and to the aqueous solution. The pump may be an inline pump or a syringe pump.

Typically, the mixing chamber may be connected to 2 to about 20 aqueous solution inlet ports. Preferably, there may be from 3 to about 11 such ports, from 3 to about 12 such ports. More preferably, there are from 3 to about 10, or from 3 to about 7 aqueous solution entry inlet ports. The mixing chamber may also be connected to from 1 to about 5 lipid solution inlet ports. Preferably, there are from 1 to about 3 lipid solution inlet ports. Most preferably, there is 1 or 2 lipid solution inlet ports. In a preferred embodiment, the mixing chamber is connected to at least 1 (e.g., 1, 2, 3, 4, or 5) lipid solution port(s) and at least 2 (e.g., 2, 3, 4, 5, 6, 7, 8, 9, 10, 11, 12, 13, 14, 15, 16, 17, 18, 19 or 20) aqueous solution inlet ports.

The mixing chamber can be further connected to 1 to about 3 outlet ports for the liposome solution for particle size control, preferably, there is 1 (e.g., 1, 2, or 3) outlet port(s).

In certain aspects, the angle between the inlet ports for the lipid and aqueous solutions is from about 18° to about 180°. Preferably the angel may be from about 24° to about 180°, more preferably from about 30° to about 180°. In some embodiments, the angle between at least one lipid and at one aqueous solution inlet ports is not 180° or a substantially similar angle. For example, the angle between at least one lipid and at one aqueous solution inlet ports is about 120° or less, about 90° or less. The angle between ports is the angle at which streams of respective solutions are directed into the mixing chamber.

The lipid and aqueous solutions may have the same flow rate through the manifold. Alternatively, the solutions may have different flow rates. The flow rates for the lipid and aqueous solutions may be 1 ml/min to about 6,000 ml/min, e.g., from about 1 ml/min to about 1,500 ml/min. Preferably, the flow rates may be from about 5 ml/min to about 1,000 ml/min, e.g., from about 5 ml/min to about 400 ml/min. More preferably, the rates may be the rates may be from about 20 ml/min to about 600 ml/min or from about 10 ml/min to about 300 ml/min. In some embodiments, the flow rates are adjusted based on the size of inlet ports to obtain the desired LNP size, morphology, PDI, and manufacturing scale.

The disclosure also provides methods for preparing lipid nanoparticles (LNP), the method comprising: a) introducing i) one or more streams of a lipid solution via a first set of one or more inlet ports of a manifold and ii) one or more streams of an aqueous solution via a second set of one or more an inlet ports of the manifold, thereby mixing the lipid solution and the aqueous solution so as to produce an LNP solution; and b) recovering the LNP solution via one or more outlet ports of the manifold; wherein the angle between at least one lipid and at one aqueous solution inlet ports is not 180° or a substantially similar angle. In some aspects, at least one stream of lipid solution and at one stream of aqueous solution collide at an angle less than about 180°. Thus, in some aspects, the method does not include a T-connector.

In some embodiments, the angle between at least one lipid and at one aqueous solution inlet ports is about 120° or less, e.g., 1150 or less, 1000 or less, 90° or less, 80° or less, 72° or less, 60° or less, 45° or less, 30° or less, 18° or less,

In some embodiments, the aqueous solution in step ii) is introduced via at least two inlet ports, e.g., at least 3, 4, 5, 6, 7, 8, 9, 10, 11, 12, 13, 14, 15, 16, 17, 18, 19, 20 or more. In some embodiments, the aqueous solution in step ii) is introduced via at least 3 but no more than 11 inlet ports, e.g., at least 3 but not more than 7, at least 3 but no more than 5, at least 4 but no more than 11, at least 5 but no more than 11, at least 6 but no more than 11.

In some embodiments, at least two (e.g., 3, 4, 5, 6, 7, etc.) aqueous inlet ports and at least one (e.g., 2, 3, 4, 5, etc.) lipid solution inlet port are in the same plane.

In some embodiments, at least one (e.g., 2) outlet port is substantially perpendicular to the plane of inlet ports. In other embodiments, at least one (e.g., 2, 3, 4, 5, etc.) outlet port is substantially not perpendicular to the plane of inlet ports. In some embodiments, at least two (e.g., 3, 4, 5, 6, 7, etc.) aqueous solution inlet ports and at least one (e.g., 2, 3, 4, 5, etc.) lipid solution inlet port are not in the same plane. In some embodiments, the aqueous solution introduced into at least one of the inlet ports differs from a second aqueous solution introduced into another inlet port.

In some embodiments, the aqueous solution and/or the lipid solution comprises an active pharmaceutical ingredient (API).

In some embodiments, step a) further comprises introducing iii) one or more streams of non-aqueous solutions via one or more inlet ports of the manifold.

### Methods of Producing Polypeptides in Cells

The present disclosure provides methods of producing a polypeptide of interest in a mammalian cell. Methods of producing polypeptides involve contacting a cell with a nanoparticle composition including a nucleic acid (e.g., mRNA) encoding the polypeptide of interest. Upon contacting the cell with the nanoparticle composition, the nucleic acid (e.g., mRNA) may be taken up and translated in the cell to produce the polypeptide of interest.

In general, the step of contacting a mammalian cell with a nanoparticle composition including a nucleic acid (e.g., mRNA) encoding a polypeptide of interest may be performed in vivo, ex vivo, in culture, or in vitro. The amount of nanoparticle composition contacted with a cell, and/or the amount of nucleic acid (e.g., mRNA) therein, may depend on the type of cell or tissue being contacted, the means of administration, the physiochemical characteristics of the nanoparticle composition and the nucleic acid (e.g., mRNA), e.g., size, charge, and chemical composition therein, and other factors. In general, an effective amount of the nanoparticle composition will allow for efficient polypeptide production in the cell.

Metrics for efficiency may include polypeptide translation (indicated by polypeptide expression), level of nucleic acid (e.g., mRNA) degradation, and immune response indicators.

The step of contacting a nanoparticle composition including a nucleic acid (e.g., mRNA) with a cell may involve or cause transfection. A phospholipid including in the lipid component of a nanoparticle composition may facilitate transfection and/or increase transfection efficiency, for example, by interacting and/or fusing with a cellular or intracellular membrane. Transfection may allow for the translation of the nucleic acid (e.g., mRNA) within the cell.

In some embodiments, the nanoparticle compositions described herein may be used as therapeutic agents. For example, a nucleic acid (e.g., mRNA) included in a nanoparticle composition may encode a therapeutic polypeptide (e.g., in a translatable region) and produce the therapeutic polypeptide upon contacting and/or entry (e.g., transfection) into a cell. In other embodiments, a nucleic acid (e.g., mRNA) included in a nanoparticle composition of the disclosure may encode a polypeptide that may improve or increase the immunity of a subject. For example, a nucleic acid (e.g., mRNA) may encode a granulocyte-colony stimulating factor or trastuzumab.

In certain embodiments, a nucleic acid (e.g., mRNA) included in a nanoparticle composition of the disclosure may encode a recombinant polypeptide that may replace one or more polypeptides that may be substantially absent in a cell contacted with the nanoparticle composition. The one or more substantially absent polypeptides may be lacking due to a genetic mutation of the encoding gene or a regulatory pathway thereof. Alternatively, a recombinant polypeptide produced by translation of the nucleic acid (e.g., mRNA) may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In another alternative, a recombinant polypeptide produced by translation of the nucleic acid (e.g., mRNA) may indirectly or directly antagonize the activity of a biological moiety present in, on the surface of, or secreted from the cell. Antagonized biological moieties may include, but are not limited to, lipids (e.g., cholesterol), lipoproteins (e.g., low density lipoprotein), nucleic acids, carbohydrates, and small molecule toxins. Recombinant polypeptides produced by translation of the nucleic acid (e.g., mRNA) may be engineered for localization within the cell, such as within a specific compartment such as the nucleus, or may be engineered for secretion from the cell or for translocation to the plasma membrane of the cell.

In some embodiments, contacting a cell with a nanoparticle composition including a nucleic acid (e.g., mRNA) may reduce the innate immune response of a cell to an exogenous nucleic acid. A cell may be contacted with a first nanoparticle composition including a first amount of a first exogenous nucleic acid (e.g., mRNA) including a translatable region and the level of the innate immune response of the cell to the first exogenous nucleic acid (e.g., mRNA) may be determined. Subsequently, the cell may be contacted with a second composition including a second amount of the first exogenous nucleic acid (e.g., mRNA), the second amount being a lesser amount of the first exogenous nucleic acid (e.g., mRNA) compared to the first amount. Alternatively, the second composition may include a first amount of a second exogenous nucleic acid (e.g., mRNA) that is different from the first exogenous nucleic acid (e.g., mRNA). The steps of contacting the cell with the first and second compositions may be repeated one or more times.

Additionally, efficiency of polypeptide production (e.g., translation) in the cell may be optionally determined, and the cell may be re-contacted with the first and/or second composition repeatedly until a target protein production efficiency is achieved.

### Methods of Delivering Nucleic Acids to Cells

The present disclosure provides methods of delivering a nucleic acid (e.g., mRNA) to a mammalian cell. Delivery of a nucleic acid (e.g., mRNA) to a cell involves administering a nanoparticle composition including the nucleic acid (e.g., mRNA) to a subject, where administration of the composition involves contacting the cell with the composition. Upon contacting the cell with the nanoparticle composition, a translatable nucleic acid (e.g., mRNA) may be translated in the cell to produce a polypeptide of interest. However, nucleic acid (e.g., mRNA)s that are substantially not translatable may also be delivered to cells. Substantially non-translatable nucleic acid (e.g., mRNA)s may be useful as vaccines and/or may sequester translational components of a cell to reduce expression of other species in the cell.

In some embodiments, a nanoparticle composition of the disclosure may target a particular type or class of cells (e.g., cells of a particular organ or system thereof). For example, a nanoparticle composition including a nucleic acid (e.g., mRNA) of interest may be specifically delivered to a mammalian kidney. Specific delivery to a particular class of cells, an organ, or a system or group thereof implies that a higher proportion of nanoparticle compositions including nucleic acid (e.g., mRNA) are delivered to the destination (e.g., tissue) of interest relative to other destinations, e.g., upon administration of a nanoparticle composition to a mammal. In some embodiments, specific delivery may result in a greater than 2 fold, 5 fold, 10 fold, 15 fold, or 20 fold increase in the amount of nucleic acid (e.g., mRNA) per 1 g of delivered to the targeted destination (e.g., tissue of interest, such as a kidney) as compared to another destination (e.g., the liver). In particular embodiments, the tissue of interest is a kidney. In certain embodiments, the tissue of interest is the vascular endothelium of a kidney. In other embodiments, the tissue of interest is selected from the group consisting of vascular endothelium in vessels (e.g., intra-coronary or intra-femoral) and tumor tissue (e.g., via intratumoral injection).

As another example of targeted or specific delivery, a nucleic acid (e.g., mRNA) that encodes a protein-binding partner (e.g., an antibody or functional fragment thereof, a scaffold protein, or a peptide) or a receptor on a cell surface may be included in a nanoparticle composition. A nucleic acid (e.g., mRNA) may additionally or instead be used to direct the synthesis and extracellular localization of lipids, carbohydrates, or other biological moieties. Alternatively, other elements (e.g., lipids or ligands) of a nanoparticle composition may be selected based on their affinity for particular receptors (e.g., low density lipoprotein receptors) such that a nanoparticle composition may more readily interact with a target cell population including the receptors. For example, ligands may include, but are not limited to, members of a specific binding pair, antibodies, monoclonal antibodies, Fv fragments, single chain Fv (scFv) fragments, Fab′ fragments, F(ab′)₂ fragments, single domain antibodies, camelized antibodies and fragments thereof, humanized antibodies and fragments thereof, and multivalent versions thereof; multivalent binding reagents including mono- or bi-specific antibodies such as disulfide stabilized Fv fragments, scFv tandems, diabodies, tridobdies, or tetrabodies; and aptamers, receptors, and fusion proteins.

In some embodiments, a ligand may be a surface-bound antibody, which can permit tuning of cell targeting specificity. This is especially useful since highly specific antibodies can be raised against an epitope of interest for the desired targeting site. In one embodiment, multiple antibodies are expressed on the surface of a cell, and each antibody can have a different specificity for a desired target. Such approaches can increase the avidity and specificity of targeting interactions.

A ligand can be selected, e.g., by a person skilled in the biological arts, based on the desired localization or function of the cell. For example an estrogen receptor ligand, such as tamoxifen, can target cells to estrogen-dependent breast cancer cells that have an increased number of estrogen receptors on the cell surface. Other non-limiting examples of ligand/receptor interactions include CCR1 (e.g., for treatment of inflamed joint tissues or brain in rheumatoid arthritis, and/or multiple sclerosis), CCR7, CCR8 (e.g., targeting to lymph node tissue), CCR6, CCR9. CCR10 (e.g., to target to intestinal tissue), CCR4, CCR10 (e.g., for targeting to skin), CXCR4 (e.g., for general enhanced transmigration), HCELL (e.g., for treatment of inflammation and inflammatory disorders, bone marrow), Alpha4beta7 (e.g., for intestinal mucosa targeting), and VLA-4NCAM-1 (e.g., targeting to endothelium). In general, any receptor involved in targeting (e.g., cancer metastasis) can be harnessed for use in the methods and compositions described herein.

Targeted cells may include, but are not limited to, hepatocytes, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells.

In particular embodiments, a nanoparticle composition of the disclosure may target hepatocytes. Apolipoprotiens such as apolipoprotein E (apoE) have been shown to associate with neutral or near neutral lipid-containing nanoparticle compositions in the body, and are known to associate with receptors such as low-density lipoprotein receptors (LDLRs) found on the surface of hepatocytes. Thus, a nanoparticle composition including a lipid component with a neutral or near neutral charge that is administered to a subject may acquire apoE in a subject's body and may subsequently deliver nucleic acid (e.g., mRNA) to hepatocytes including LDLRs in a targeted manner.

Nanoparticle compositions of the disclosure may be useful for treating a disease, disorder, or condition characterized by missing or aberrant protein or polypeptide activity.

Upon delivery of a nucleic acid (e.g., mRNA) encoding the missing or aberrant polypeptide to a cell, translation of the nucleic acid (e.g., mRNA) may produce the polypeptide, thereby reducing or eliminating an issue caused by the absence of or aberrant activity caused by the polypeptide. Because translation may occur rapidly, the methods and compositions of the disclosure may be useful in the treatment of acute diseases, disorders, or conditions such as sepsis, stroke, and myocardial infarction. A nucleic acid (e.g., mRNA) included in a nanoparticle composition of the disclosure may also be capable of altering the rate of transcription of a given species, thereby affecting gene expression.

Diseases, disorders, and/or conditions characterized by dysfunctional or aberrant protein or polypeptide activity for which a composition of the disclosure may be administered include, but are not limited to, cancer and proliferative diseases, genetic diseases (e.g., cystic fibrosis), autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. A specific example of a dysfunctional protein is the missense mutation variants of the cystic fibrosis transmembrane conductance regulator (CFTR) gene, which produce a dysfunctional protein variant of CFTR protein, which causes cystic fibrosis. The present disclosure provides a method for treating such diseases, disorders, and/or conditions in a subject by administering a nanoparticle composition including a nucleic acid (e.g., mRNA) and a lipid component including KL10, a phospholipid (optionally unsaturated), a PEG lipid, and a structural lipid, wherein the m RNA encodes a polypeptide that antagonizes or otherwise overcomes an aberrant protein activity present in the cell of the subject.

In some embodiments, a nanoparticle composition may be designed and administered to promote transient expression of a polypeptide in a particular organ or system thereof. Transient expression may be useful as a therapeutic treatment on its own or in combination with other therapeutic treatments including drug administration and/or surgical procedures. In particular, transient expression of vascular endothelial growth factor A (VEG F-A) may be promoted by administration of a nanoparticle composition of the disclosure. VEG F-A plays an important role in angiogenesis, particularly in the vascular endothelium. Though over-expression of VEGF-A has been associated with a variety of cancers, transient expression of VEG F-A in, for example, renovascular structures, may reduce arterial stenting to treat vascular diseases such as atherosclerotic renovascular disease (ARVD). Nanoparticle compositions including m RNA encoding VEGF-A and optimized to selectively deliver nucleic acid (e.g., mRNA) to the renal system (e.g., a kidney) may thus be useful in the treatment of ARVD and related disorders.

The disclosure provides methods involving administering nanoparticle compositions including nucleic acid (e.g., mRNA) or pharmaceutical compositions including the same. Compositions of the disclosure, or imaging, diagnostic, or prophylactic compositions thereof, may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject; the purpose of the administration; the particular composition; the mode of administration; and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment. The specific therapeutically effective, prophylactically effective, or otherwise appropriate dose level (e.g., for imaging) for any particular patient will depend upon a variety of factors including the severity and identify of a disorder being treated, if any; the one or more nucleic acid (e.g., mRNA)s employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

A nanoparticle composition including one or more nucleic acids (e.g., mRNAs) may be administered by any route. In some embodiments, compositions of the disclosure, including prophylactic, diagnostic, or imaging compositions including one or more nanoparticle compositions of the disclosure, are administered by one or more of a variety of routes, including oral, intravenous, intramuscular, intra-arterial, intramedullary, intrathecal, subcutaneous, intraventricular, trans- or intra-dermal, interdermal, rectal, intravaginal, intraperitoneal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermal\hat{ }, intra-arterially, intratumorally, subcutaneously, or by inhalation. However, the present disclosure encompasses the delivery of compositions of the disclosure by any appropriate route taking into consideration likely advances in the sciences of drug delivery. In general, the most appropriate route of administration will depend upon a variety of factors including the nature of the nanoparticle composition including one or more m RNAs (e.g., its stability in various bodily environments such as the bloodstream and gastrointestinal tract), the condition of the patient (e.g., whether the patient is able to tolerate particular routes of administration), etc.

In certain embodiments, compositions in accordance with the present disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of a composition in a given dose, where a dose of 1 mg/kg provides 1 mg of a composition per 1 kg of subject body weight. In particular embodiments, a dose of about 0.001 mg/kg to about 10 mg/kg of a nanoparticle composition of the disclosure may be administrated. In other embodiments, a dose of about 0.005 mg/kg to about 2.5 mg/kg of a nanoparticle composition may be administered. In certain embodiments, a dose of about 0.1 mg/kg to about 1 mg/kg may be administered. In other embodiments, a dose of about 0.05 mg/kg to about 0.25 mg/kg may be administered. A dose may be administered one or more times per day, in the same or a different amount, to obtain a desired level of nucleic acid (e.g., mRNA) expression and/or therapeutic, diagnostic, prophylactic, or imaging effect. The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition.

Nanoparticle compositions including one or more nucleic acids (e.g., mRNAs) may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more nanoparticle compositions including one or more different m RNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the disclosure, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

### EXAMPLES

The invention is further described in the following examples, which do not limit the scope of the invention described in the claims.

The experiments started by adding a polymer (e.g., L121) into reference LNP formulations, to test whether the polymer can improve the mRNA delivery to cells. As significant improvements were observed (e.g., as shown in Examples 1-3), further experiments were designed to test additional formulations containing various types of polymers, to optimize LNP formulations with significantly improved RNA/DNA delivery efficiency combined with substantially reduced mol % of ionizable lipid and increased mRNA loading as indicated by the dramatically reduced N/P ratio.

### Example 1. GFP Cell Fluorescence Images: The Visualized Improvement of mRNA Delivery by Adding the Polymer to the Reference LNPs

The in vitro transfections of GFP (green fluorescent protein) mRNA by different LNPs were tested. Reference LNPs (Formulation codes: CPT101, CPT103, CPT105, and CPT107) were generated to have the molar ratios of the mRNA COVID-19 vaccines, e.g., 50 mol % cationic (or ionizable) lipid, 10 mol % DSPC (distearoylphosphatidylcholine), 38.5 mol % cholesterol, and 1.5 mol % PEG2000-DSPE. A triblock copolymer of Pluronic™ surfactant, L121, was also included in the formulations to generate corresponding Polymer-Advanced Lipid Nanoparticles, or PALNPs (Formulation codes: CPT067, CPT069, CPT106, and CPT108), respectively. The molar percentage (mol %) of the LNPs are shown in the table below.

TABLE 1
Formulations (molar ratio) of PALNPs and reference LNPs

| | | Cationic (ionizable) Lipids | | | | | | | | |
|---|---|---|---|---|---|---|---|---|---|---|
| | | | DLin- | Dlin- | | | | | | |
| Formulation | PALNP/ | ALC- | MC3- | KC2- | DLin- | | | PEG2000- | | N/P |
| Code | reference | 0315 | DMA | DMA | DMA | DSPC | Cholesterol | DSPE | L121 | Ratio |
| CPT067 | PALNP/ | | | | 41.5 | 10 | 38.5 | 1.5 | 5 | 6 |
| CPT101 | reference | | | | 50 | 10 | 38.5 | 1.5 | | 6 |
| CPT069 | PALNP/ | | | 41.5 | | 10 | 41.5 | 2 | 5 | 6 |
| CPT104 | PALNP/ | | | 45 | | 10 | 38.5 | 1.5 | 5 | 6 |
| CPT103 | reference | | | 50 | | 10 | 38.5 | 1.5 | | 6 |
| CPT106 | PALNP/ | | 45 | | | 10 | 38.5 | 1.5 | 5 | 6 |
| CPT105 | reference | | 50 | | | 10 | 38.5 | 1.5 | | 6 |
| CPT108 | PALNP/ | 45 | | | | 10 | 38.5 | 1.5 | 5 | 6 |
| CPT107 | reference | 50 | | | | 10 | 38.5 | 1.5 | | 6 |

The PALNPs shared the same cationic lipid with the corresponding control (reference) LNP, while L121 was also included in the formulation. The cationic lipids selected for this study were ACL-0315, DLin-MC3-DMA, DLin-KC2-DMA, and DLin-DMA. All the LNPs were prepared using the nPort™ technology as disclosed in U.S. Pat. No. 11,497,715 B2.

L121 is a triblock copolymer of Pluronic™ surfactant consisting of a central hydrophobic block of polypropylene glycol (PPO) flanked by two hydrophilic blocks of polyethylene glycol (PEO). The general formula of this Pluronic™ surfactant is shown below.

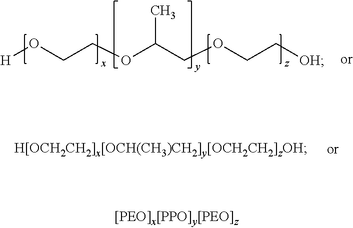

For L121, x=5, Y=68, z=5, and the average molecular weight is about 4,400 Daltons. The total weight/weight (w/w) ratio of the two PEO blocks is about 10% of the molecule.

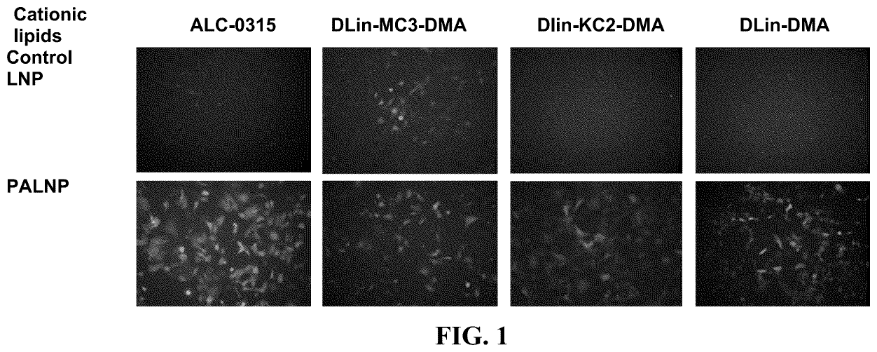

HEK293, A549, and HepG2 cell lines were used to test the in vitro transfections of the GFP mRNA (as a reporter gene) via the above described LNPs. Specifically, the cells were cultured in black 96-well plates with cover glass bottoms. The cell fluorescence images were taken by a Nikon SLR digital camera that was mounted to a Nikon TE-2000U microscope equipped with high resolution plan apochromat (APO) objectives. FIG. 1 shows the representative GFP fluorescence images of HEK293 cells transfected by the PALNPs (CPT108, CPT106, CPT069, and CPT67, respectively) with 0.1 μg mRNA per well (bottom row), and HEK293 cells transfected by control LNPs (CPT107, CPT105, CPT103, and CPT101, respectively) with 0.6 μg mRNA (upper row). The N/P ratio was 6.0 for all LNPs. Bright Green fluorescence was detected from the majority of the PALNP-treated cells, whereas among the four control LNPs, only CPT105 LNP (containing DLin-MC3-DMA) induced moderate fluorescence while the remaining three were significantly fainter. The results indicate that L121 significantly enhanced the transfection efficiency of GFP mRNA.

### Example 2. Improved mRNA Delivery Determined by FACS Analysis

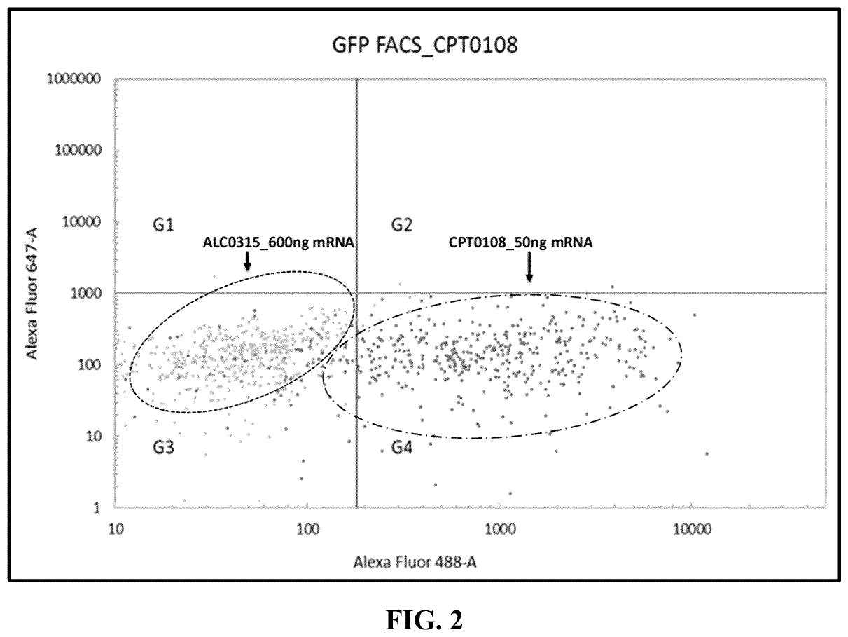

FACS (fluorescence activated cell sorting) was used for quantitative studies of GFP mRNA transfection. Several leading PALNPs made of cationic lipids (ALC-0315, DLin-MC3-DMA, DLin-KC2-DMA, or DLin-DMA) were analyzed by FACS and compared against their control LNPs. Specifically, 0.05, 0.10, 0.20, or 0.4 μg GFP mRNA was loaded into each PALNP, while 0.6 μg GFP mRNA was loaded into the corresponding control LNPs. FIG. 2 shows the FACS results of CPT108, which contained ALC-0315 as the cationic lipid. Both the fluorescence dots representing cells transfected by CPT108 with 0.05 μg mRNA, and the fluorescence dots representing cells transfected by the control LNP (CPT107) with 0.6 μg mRNA are indicated by circles.

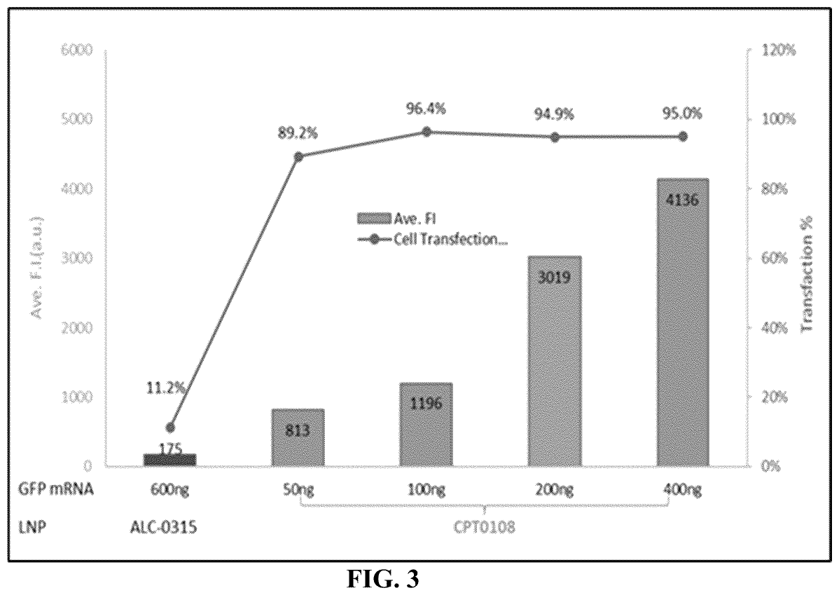

About 11% of the cells transfected by CPT107 (control) were very weakly GFP positive with an average fluorescence intensity of about 175 (arbitrary unit, (a.u.)), whereas 89% of cells transfected by CPT108 (loaded with only one twelfth of mRNA) were GFP positive with an approximately 5-fold higher average GFP fluorescence than the control. As shown in FIG. 3, when mRNA was increased to 0.4 μg with CPT108, over 95% cells emitted GFP signals, and the fluorescence intensity was about 24-fold higher than the control.

### Example 3. Improvement of Cell Transfection Efficiency by FACS Analysis

The five types of PALNPs in Table 1 were loaded with 0.05 μg GFP mRNA, and the cell transfection efficiency was determined to be about 76-94% with fluorescence intensity (mean) ranging from 660-3000 (a.u.). When the mRNA dose was increased to 0.4 μg, the cell transfection efficiency remained steady over 95% with 4000-10600 (a.u.) fluorescence intensity. In contrast, when the corresponding reference LNPs were loaded with 0.6 μg mRNA, only 10-60% transfection efficiency and 175-788 (a.u.) intensity were observed, as shown in the fluorescence intensity table below.

TABLE 2
Summary of FACS analysis

| LNP | Reference LNP | PALNP | |
|---|---|---|---|
| mRNA (μg) | 0.6 | 0.05 | 0.4 |
| Cell Transfection (%) | 10-60% | 76-94% | >95% |
| GFP Intensity (mean, a.u.) | 175-788 | 660-3000 | 4000-10600 |

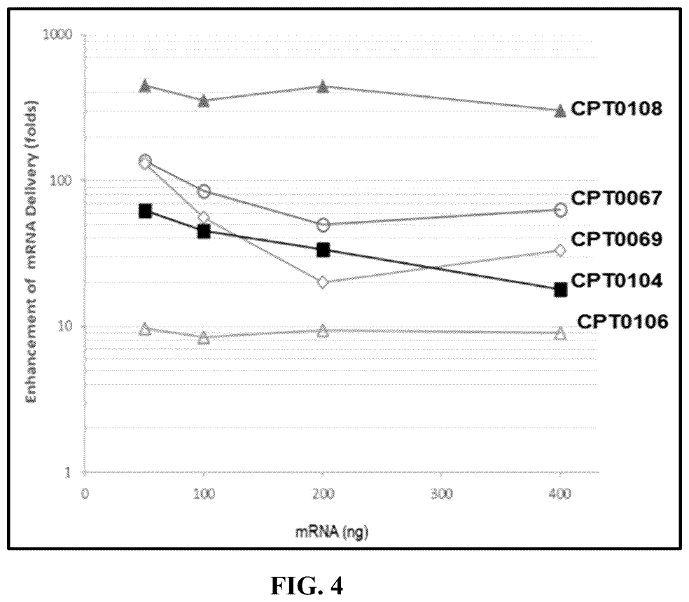

As shown in the table below and FIG. 4, combining all the significantly improved transfection factors including lower mRNA dose, higher cell transfection rate, and much higher protein expression level in the PALNP groups, the PALNPs exhibited an increased overall mRNA delivery efficiency that is about 10-445 folds higher than the control LNPs.

TABLE 3
mRNA delivery enhancement of PALNPs

| PALNP vs. Reference LNP | | | |
|---|---|---|---|
| | Reference | Cationic (ionizable) | mRNA Delivery Enhancement |
| PALNP | LNP | Lipids | (folds) |
| CPT108 | CPT107 | ALC-0315 | 300-445 |
| CPT067 | CPT101 | DLin-DMA | 60-135 |
| CPT104 | CPT103 | DLin-KC2-DMA | 30-130 |
| CPT069 | CPT103 | DLin-KC2-DMA | 20-60 |
| CPT106 | CPT105 | Dlin-MC3-DMA | 10 |

### Example 4. Enhanced mRNA Transfection Efficiency at Low N/P Ratios and Low mRNA Doses

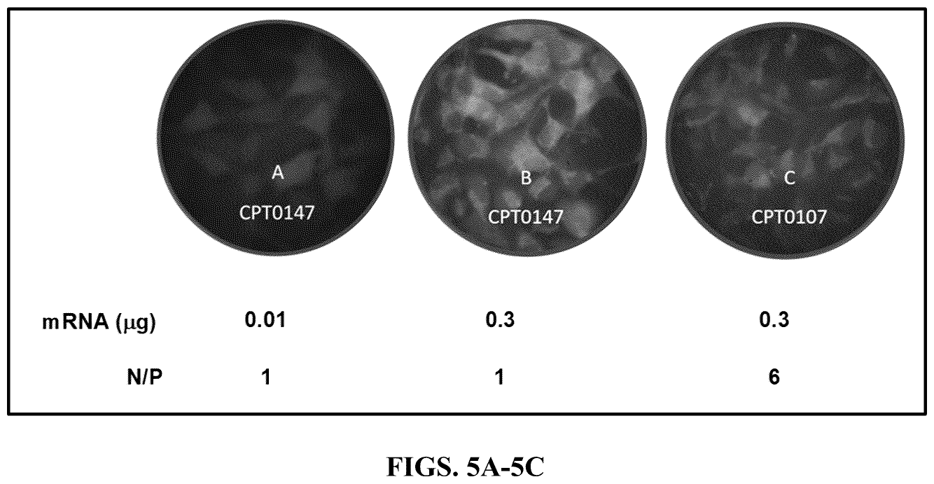

The results above demonstrated that the addition of Pluronic™ polymers improved the transfection efficiency of the LNPs dramatically, indicating a potential for developing LNPs with improved properties. In reference LNPs, excessive cationic lipids are required for RNA/DNA encapsulation and for LNP escaping from lysosomes. The severe toxicities from the high dose of cationic lipids have been a huge barrier in LNP-based gene delivery. To solve this problem, highly efficient LNP formulations were developed to reduce both the N/P ratio and the dose of mRNA. For example, in CPT147 LNP, the N/P ratio was reduced to 1, meaning 6-fold less cationic lipid can be used in the formulation delivering the same amount of mRNA/DNA. Compared to the reference or control LNP (CPT107) with 0.3 μg mRNA and N/P=6 (FIG. 5C), CPT147 LNP induced comparable GFP fluorescence with 0.01 μg mRNA and N/P=1 (FIG. 5A). That was a 30-fold reduction in RNA dose (0.01 μg versus 0.3 μg), and thus a 180-fold reduction of cationic lipid ALC-0315. When 0.3 μg mRNA was used, CPT147 LNP induced oversaturated GFP fluorescence in the cells (FIG. 5B). The formulation of CPT147 LNP is shown in the table below.

TABLE 4
The formulations (mol %) of CPT147 LNP

| Ingredient | ALC-0315 | DSPC | DOPE | Cholesterol | PEG2000-DSPE | L121 | N/P |
|---|---|---|---|---|---|---|---|
| Mol % | 10 | 22.5 | 22.5 | 38.5 | 1.5 | 5 | 1 |

To track the uptake of LNPs by the cells, the LNPs were labeled with Rhodamine-DSPC (Rhd-DSPC), the punctuated red fluorescence in the images of FIGS. 5B-5C was from Rhodamine-DSPC. In FIG. 5A, the lipid concentration was too low (1/180 of the control) to generate detectable red fluorescence by the microscope.

### Example 5. Determination of Optimized N/P Ratio for GFP Expression

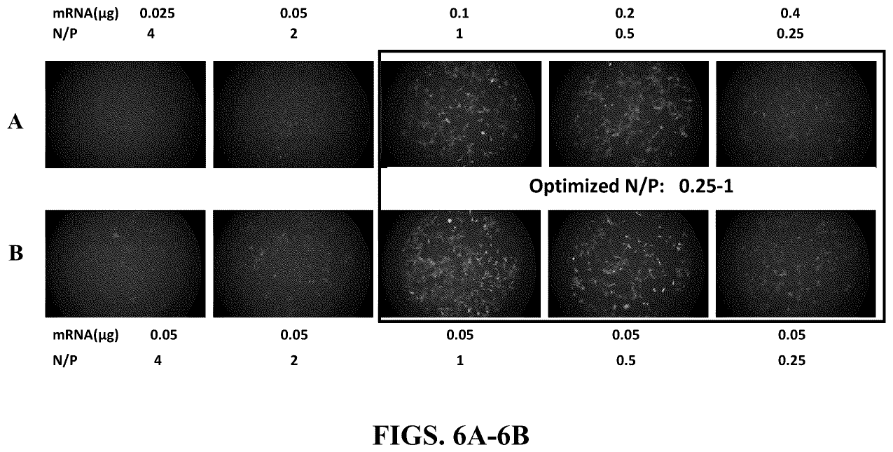

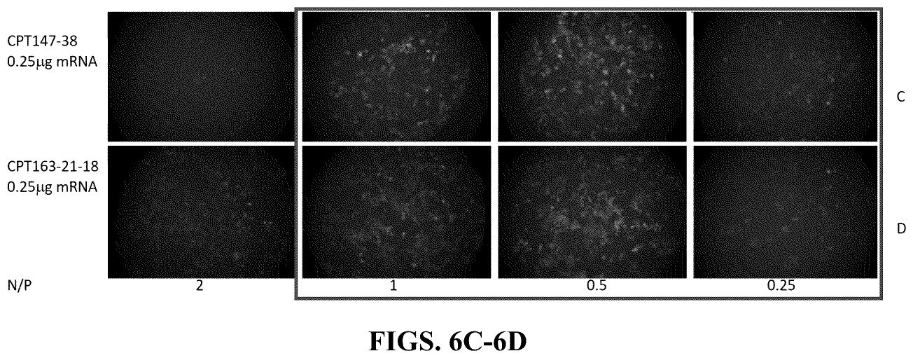

The optimized N/P ratio was determined for CPT147 LNP from two opposite approaches. As shown in FIG. 6A, LNP quantity was fixed for all the wells (1 μl) while different amounts of mRNA were used, resulting in various N/P ratios in the range of 0.25-4. In FIG. 6B, mRNA was fixed at 0.05 μg, but varying LNP quantities were used for each well to make desirable N/P ratios from 0.25 to 4. The brightest fluorescence of the cells appeared in groups with an N/P ratio between 0.5 and 1. Sharp declines of GFP expression was observed when the N/P ratio was increased to 2 or decreased to 0.25. Therefore, the optimized N/P ratio of CPT147 LNP is 0.25-1, which is about 6-24 times lower than the reference LNPs.

In a separate study, the N/P ratio effects on the GFP mRNA transfection were determined using CPT147-38 LNP (FIG. 6C) and CPT163-21-18 LNP (FIG. 6D). The formulations of CPT147-38 and CPT147 are the same while the ratios of ALC-0315 and L121 are dramatically different from each other. The lipid ratios of LNPs CPT163-21-18 and CPT147-38 are similar, but the block polymers are different. Interestingly, despite of the differences in the formulations and/or the lipid ratios, these LNPs shared a similarly optimized N/P ratio, ranging from 0.25 to 1.

TABLE 5
The formulations (mol %)of the LNPs tested for N/P ratio optimization

| | ALC-0315 | DSPC | DOPE | Cholesterol | PEG2000-DSPE | L121 | L81 |
|---|---|---|---|---|---|---|---|
| CPT147 | 10 | 22.5 | 22.5 | 38.5 | 1.5 | 5 | |
| CPT147-38 | 18 | 20 | 20 | 34 | 1.5 | 6.8 | |
| CPT163-21-18 | 19 | 21 | 21 | 36 | 1.4 | | 1.8 |

### Example 6. Fast Cell Uptake and Protein Expression with PALNP

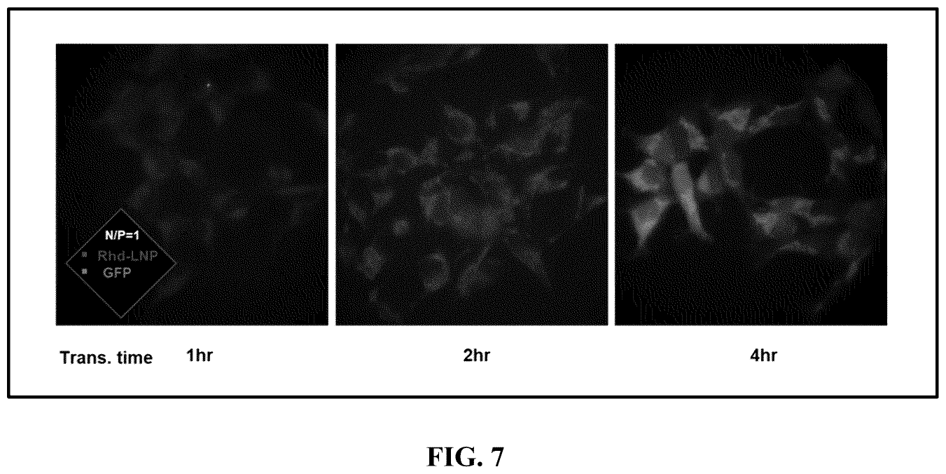

Cell uptake of the PALNPs was determined. Specifically, red fluorescence emitted from the rhodamine-DSPC labeled LNP (Rhd-LNP) was observed in the cell plasma membranes just minutes after adding CPT147 LNP to the cells. Such fast uptake by the cells promoted fast mRNA delivery to the cytosol, resulting in a fast expression of the GFP protein. As shown in FIG. 7, strong GFP fluorescence was observed 4 hours after CPT147 LNP (N/P=1, mRNA 0.2 μg) were added to the cells.

Also in FIGS. 5A-5C, the molar ratio of ALC-0315 to Rhd-DSPC was identical in both CPT-0147 and the control LNP (CPT107). Due to the low N/P ratio, the Rhd-DSPC quantity used in the cell sample of FIG. 5B was only one sixth of that used in the control sample (FIG. 5C). However, the red fluorescence intensity of FIG. 5B was comparable to that of FIG. 5C, indicating that CPT147 improved cell uptake about six times more efficiently than the control LNP (CPT107).

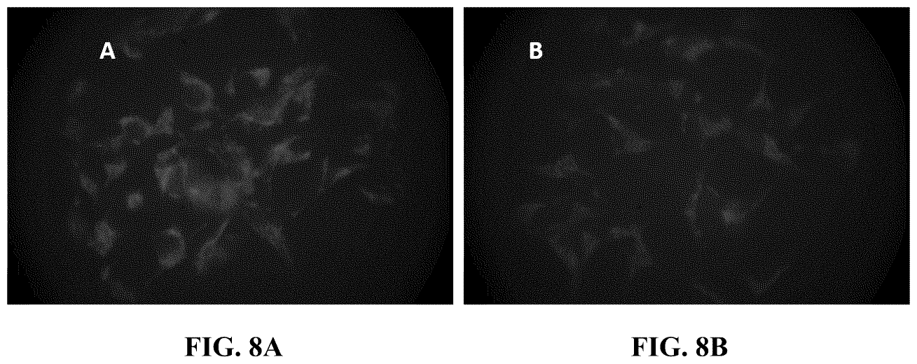

In another experiment, cells were transfected by Rhodamine-DSPC labeled CPT147 containing 0.25 μg mRNA (FIG. 8A) or the control LNP (CPT107) containing cationic lipid ALC-0315 and 0.25 μg mRNA (FIG. 8B). The total lipids and Rhodamine-DSPC of the LNP used in FIG. 8A was one sixth of those in the LNP used in FIG. 8B due to the low N/P ratio of CPT147. However, the red fluorescence shown in FIG. 8A was significantly stronger than that in FIG. 8B, indicating that CPT147 underwent a much faster cell uptake rate than the control LNP.

### Example 7. Transient mRNA Transfection by PALNP is Sufficient for Protein Expression

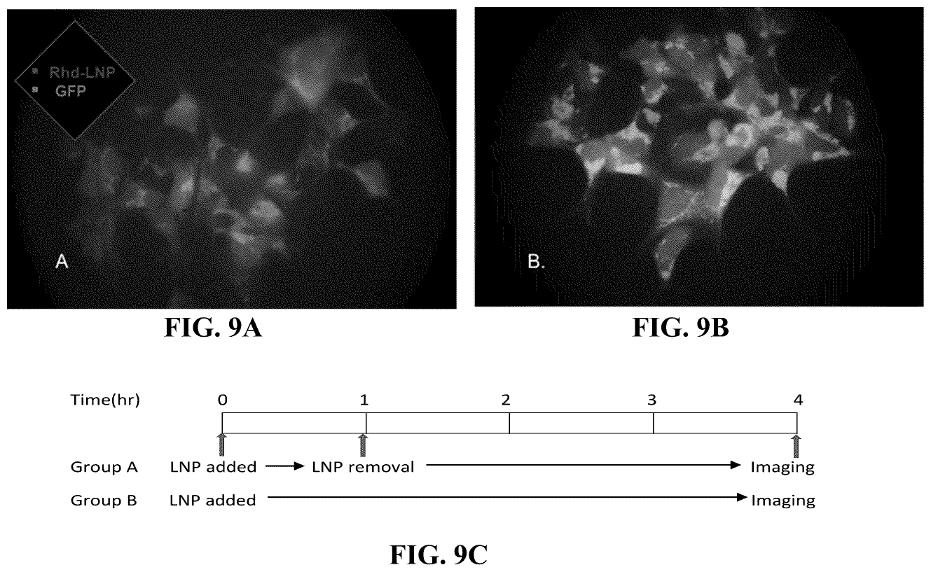

The high mRNA transfection efficiency can be further demonstrated by the observation that even transient mRNA transfection is sufficient for massive GFP expression. In this study, CPT147 LNP was removed from the well via medium exchange one hour after transfection (FIG. 9A), followed by an additional 3-hour culture in a fresh medium. Alternatively, the cells were cultured for 4 hours without LNP removal (FIG. 9B). The resulting images were taken four hours after the transfection started (FIG. 9C). The bright fluorescence of the cells in FIG. 9A demonstrates that within one hour of transfection, CPT147 LNP delivered sufficient mRNA into the cells for protein expression. FIG. 9B shows that longer exposure to mRNA resulted in more LNP uptake by the cells (as observed by the stronger red fluorescence) and greater protein expression (as observed by the brighter green fluorescence). The results indicate that transient exposure (1 hour) of the cells to is sufficient for protein expression using CPT147 LNP.

### Example 8. Reduced Cytotoxicity of PALNP

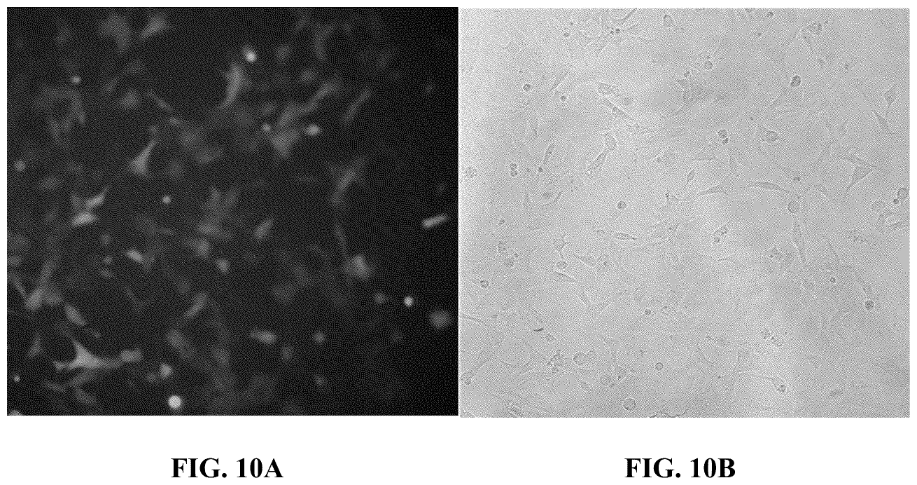

Cationic lipids are strongly cytotoxic. For example, they can disrupt cell membranes thereby causing massive cell apoptosis and hemolysis. Therefore, the cytotoxicity of cationic lipid-containing LNP brings considerable safety issues for clinical use. To test the cytotoxicity of the PALNPs, cells were transfected with CPT147 LNP for 96 hours. As shown in FIGS. 10A-10B, both a fluorescence image and the corresponding bright field image of the cells were recorded. The results indicate that the transfected cells successfully expressed GFP, and also maintained normal cell cycle, morphology and growth. Thus, no significant cytotoxicity of CPT147 LNP was observed, along with the dramatic increase in gene transfection efficiency.

### Example 9. PALNPs are Remarkably Effective for Plasmid DNA Transfection

In biopharmaceuticals, mRNA holds great advantages over DNA in terms of delivery and safety. However, it is not a replacement for DNA in gene therapies due to mRNA's disadvantages to DNA in commercial production, stability, and sustained protein expression in the transfected cells. Therefore, LNP for plasmid DNA delivery is still a huge demand for gene therapies. Due to the greater challenge for DNA delivery, after nearly 40 years of efforts, there are still no marketed DNA-based therapeutics or vaccines in the world. Extensive in vitro tests were performed on the leading LNPs for GFP plasmid DNA delivery.

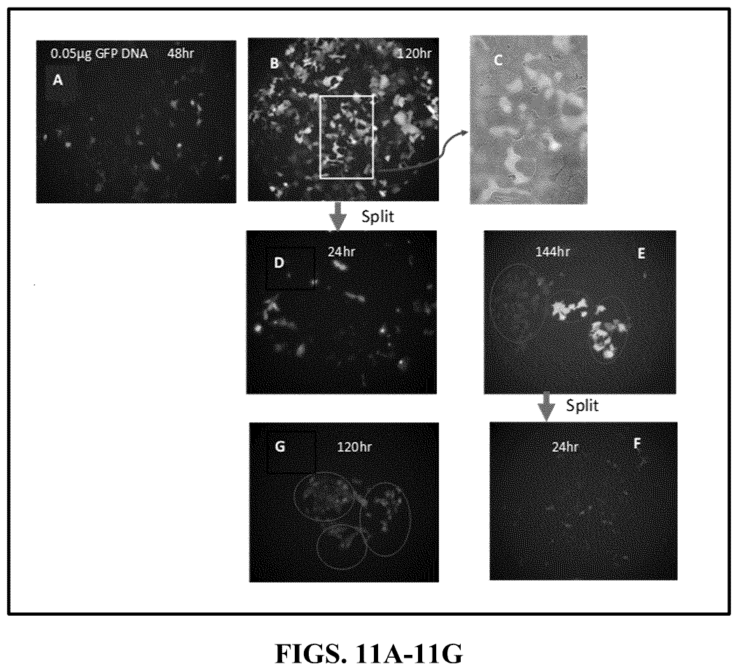

In a head-to-head comparison with the reference LNP (CPT107), CPT147 efficiently delivered plasmid DNA into the cells for protein expression. Specifically, there was no fluorescence observed from the cells transfected with reference LNPs 48 hours post-transfection, whereas cells transfected with CPT147 emitted detectable GFP fluorescence (FIG. 11A). During transfection, the cells grew normally, e.g., no cell round-up, necrosis, or apoptosis. FIGS. 11B-11C shows that, after the 120-hour transfection, 1) the cell confluency reached 100% on the plate with a normal morphology, indicating healthy growth during transfection; and 2) nearly 100% of cells emitted bright green fluorescence, indicating PALNP-mediated plasmid DNA transfection and protein expression. Furthermore, the cells in FIG. 11B were rinsed, digested by trypsin, and reseeded to grow in a fresh medium for an additional 24 hours (FIG. 11D) or 144 hours (FIG. 11E). The cells maintained a healthy growth condition, and formed colonies emitting green fluorescence (the circled cells). The cells in FIG. 11E were further rinsed, digested by trypsin, and reseeded to grow in a fresh medium for an additional 24 hours (FIG. 11F) or 120 hours (FIG. 11G). The cells maintained a healthy growth condition, and formed colonies emitting green fluorescent signals (the circled cells) again.

Overall, the above results demonstrated that the PALNPs can not only deliver mRNA but also efficiently deliver plasmid DNA into the cells. No obvious cytotoxicity was observed when cells were transfected with the PALNPs. In addition, the results indicate that plasmid DNA gene delivered can be passed from parent cells to daughter cells for many generations, and sustainable protein expression was achieved.

### Example 10. Accelerated Stability Assessment

mRNA degrades quickly in the solutions, the presence of trace amount of RNase can greatly promote mRNA degradation. Therefore, the stability of mRNA in LNP is one of the key properties to be monitored for the LNPs. Unfortunately, mRNA exhibited a poor stability in the LNP of both Pfizer's and Modema's mRNA LNP vaccines against COVID-19. For example, the former is only stable for up to 2 hours at room temperature, and the latter is only stable up to 8 hours. This poor stability has been attributed to the formation of the blebs of the reference LNPs assembled with the formulations, and part of the mRNA molecules encapsulated in the LNP were released into the aqueous phase in the blebs. Details can be found, e.g., in Mark L. B. et al., Encapsulation state of messenger RNA inside lipid Nanoparticles, Biophysical Journal 120, 2766-2770, Jul. 20, 2021.

In order to assess the stability of the PALNPs, the CPT147E LNP was prepared and stored at 25° C. for one week. The formulation of CPT147E is shown in the table below.

TABLE 6
The formulation of CPT147E LNP

| Ingredient | ALC-0315 | DSPC | DOPE | Cholesterol | PEG2000-DSPE | L121 | N/P |
|---|---|---|---|---|---|---|---|
| Mol % | 18.0 | 19.8 | 19.8 | 34.2 | 1.4 | 6.8 | 1 |

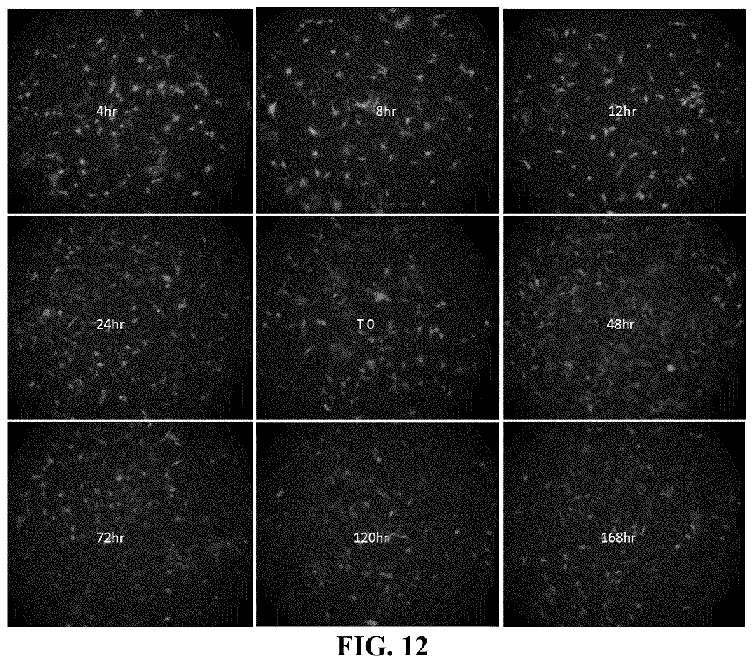

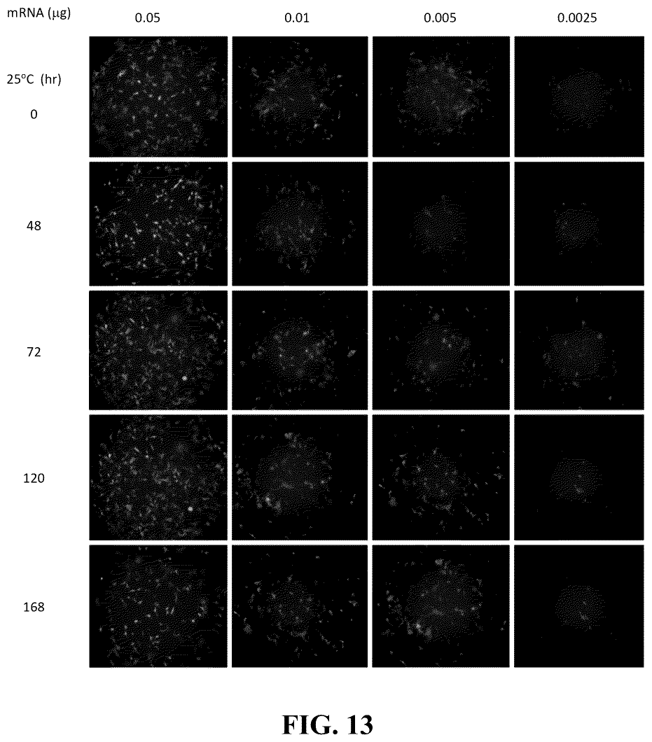

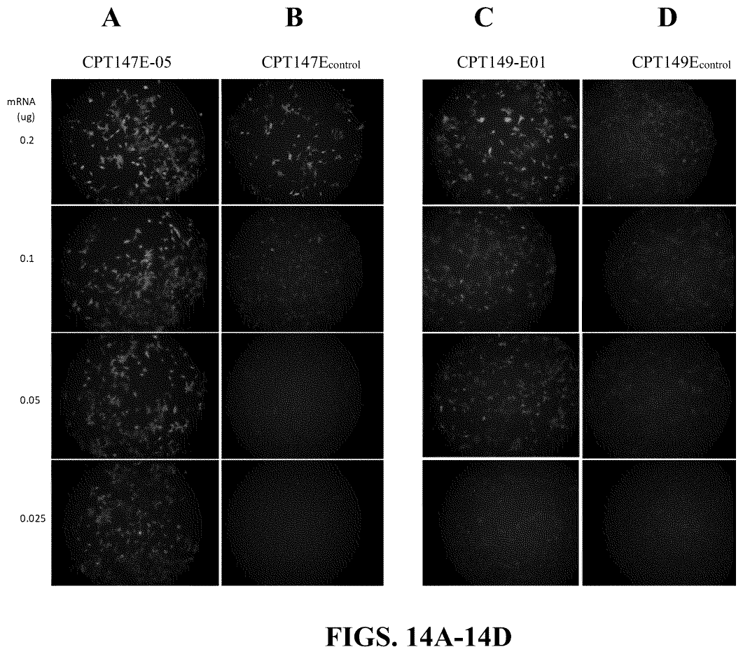

First, the GFP expression levels of the LNPs at different time points were compared. As shown in FIG. 12, no significant difference was observed in both the cell transfection percentage and cell fluorescence intensity from the samples containing 0.05 μg mRNA that were stored for different times (from time 0 (TO) to 168 hours). Second, in order to avoid mRNA saturation-induced false stability results, the cells were transfected with serial dilutions of mRNA (FIG. 13). It was observed that the cell transfection efficiency was reduced when mRNA quantity was decreased, indicating that the cells were not saturated with the mRNA. Therefore, the overall comparable cell transfection efficiency at the same mRNA concentration, as shown in FIG. 13, demonstrates that the PALNPs are stable for at least 7 days at 25° C. That is a dramatic improvement compared to the COVID-19 mRNA vaccines, which are only stable at room temperature for 2-8 hours.

### Example 11. The Block Co-Polymer Plays an Essential Role in mRNA Transfection

To demonstrate the critical roles of the co-block polymer in the LNPs for mRNA transfection, the in vitro GFP mRNA transfection efficiency by groups of LNPs made of different ionizable lipids were compared. For example, CPT147E-05, CPT149E-01, and CPT162E-01 contained ALC-0315, SM-102, and Dlin-KC2-DMA, respectively, in the presence of the triblock polymer L121. The corresponding control LNPs CPT147E_{control}, CPTI49E_{control} and CPT162E_{control} contained the same ionizable lipids, but did not contain L121.

TABLE 7
The LNP formulations (mol %) in the presence and absence of the triblock polymers

| | ALC- | SM- | DLin-KC2- | | | | PEG2000- | | |
|---|---|---|---|---|---|---|---|---|---|
| | 0315 | 102 | DMA | DSPC | DOPE | Cholesterol | DSPE | L121 | N/P |
| CPT147E-05 | 18.0 | | | 19.8 | 19.8 | 34.2 | 1.4 | 6.8 | 1 |
| CPT147Econtrol | 19.3 | | | 21.3 | 21.3 | 36.7 | 1.4 | | 1 |
| CPT149E-01 | | 18.0 | | 19.8 | 19.8 | 34.2 | 1.5 | 6.8 | 1 |
| CPT149Econtrol | | 19.3 | | 21.2 | 21.2 | 36.7 | 1.6 | | 1 |
| CPT162E-01 | | | 18.0 | 19.8 | 19.8 | 34.2 | 1.5 | 6.8 | 1 |
| CPT162Econtrol | | | 19.3 | 21.2 | 21.2 | 36.7 | 1.6 | | 1 |

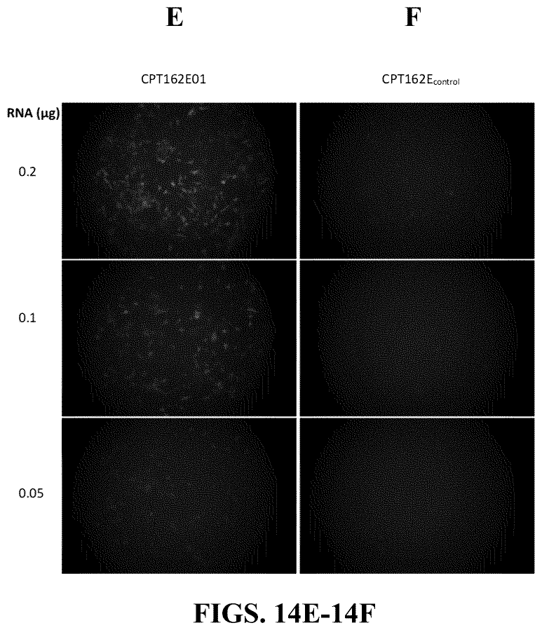

As shown in FIGS. 14A-14F, all the LNPs containing L121 exhibited an mRNA transfection efficiency that was at least 10 times higher than that of the corresponding control LNPs, which contained identical lipid compositions but in the absence of L121.

### Example 12. Generation of PALNPs Using Different Block Co-Polymers with a Wide Range of Molecular Weight

Other triblock polymers, e.g., L92 and L81, were tested to substitute L121 in PALNPs. The molecular structures of the tested triblock polymers are shown in the table below.

TABLE 8
The molecular structures of the different triblock polymers tested

| | L121 | L92 | L81 | |
|---|---|---|---|---|
| M.W.(Average) x y PEO % (w/w) | ~4,400 5 68 10% | ~3,650 8.2 50 20% | ~2,750 3.1 43 10% | 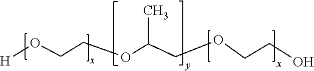 |

LNPs were generated using the method described herein, with the formulations listed in the table below.

TABLE 9
The formulations of PALNPs containing different block co-polymers

| | | | | DLin- | | | | | | | | |
|---|---|---|---|---|---|---|---|---|---|---|---|---|
| | ALC- | SM- | DLin- | KC2- | | | | PEG2000- | | | | |
| | 0315 | 102 | DMA | DMA | DSPC | DOPE | Cholesterol | DSPE | L121 | L92 | L81 | N/P |
| CPT147-04 | 10.0 | | | | 22.5 | 22.5 | 38.5 | 1.5 | 5.0 | | | 1 |
| CPT163-10 | 19.0 | | | | 20.9 | 20.9 | 36.0 | 1.4 | | | 1.9 | 1 |
| CPT161E | | | 18.0 | | 19.8 | 19.8 | 34.2 | 1.5 | 6.8 | | | 1 |
| CPT164E | | | | 18.3 | 20.3 | 20.3 | 35.1 | 1.4 | | 4.6 | | 1 |
| CPT149-01 | | 18.0 | | | 19.8 | 19.8 | 34.2 | 1.5 | 6.8 | | | 0.75 |
| CPT200-07 | | 22.3 | | | 15.6 | 15.6 | 43.0 | 1.1 | | | 2.2 | 1 |

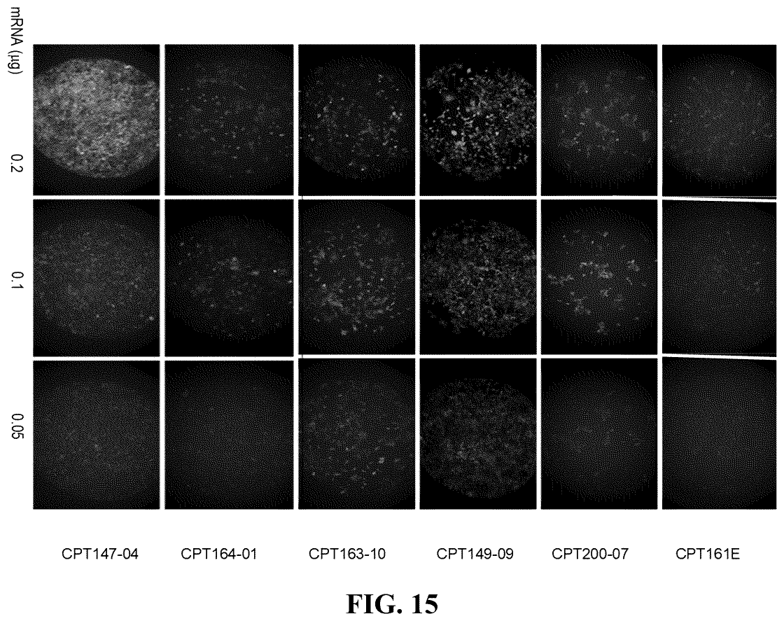

HEK293 cells were transfected using these LNPs loaded with 0.05-0.2 μg GFP mRNA, and the fluorescence images were recorded after 24 hours. As shown in FIG. 15, all the transfected cells emitted bright green fluorescent signals of GFP, indicating that not only L121, but other block co-polymers having a wide range of molecular weight can be used to generate PALNPs.

It was also found that the mRNA delivery efficiency of the LNPs containing the three Pluronic™ copolymers as in the order of L121-L81>L92. The results indicate that 1) the hydrophobicity of the polymers plays a key role in mRNA delivery, and lower weight percentage of PEO blocks correlates with a higher mRNA delivery efficiency; and 2) the molecular weight plays a minor role here as L81 is slightly less efficient than L121 while its molecular weight is significantly lower than L121.

### Example 13. Generation of PALNPs Using Permanently Charged Cationic Lipids

Dioleoyl-3-trimethylammonium propane (DOTAP) is a typical cationic lipid with a permanently positive charge. It is widely used in research for gene delivery and exhibits a concentration-dependent potent cytotoxicity.

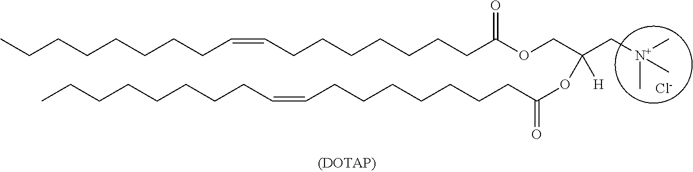 [SMILES: CCCCCCCC/C=C\CCCCCCCC(=O)OCC(C[N+](C)(C)C)OC(=O)CCCCCCC/C=C\CCCCCCCC ; [Cl-]]

CPT153E LNP were generated using the method described herein, and its formulation is listed in the table below.

TABLE 10
The formulation (Mol %) of CPT153E LNP

| | DOTAP | DSPC | DOPE | Cholesterol | PEG2000-DSPE | L121 | N/P |
|---|---|---|---|---|---|---|---|
| CPT153E | 17.9 | 19.9 | 19.9 | 34.1 | 1.4 | 6.8 | 1 |

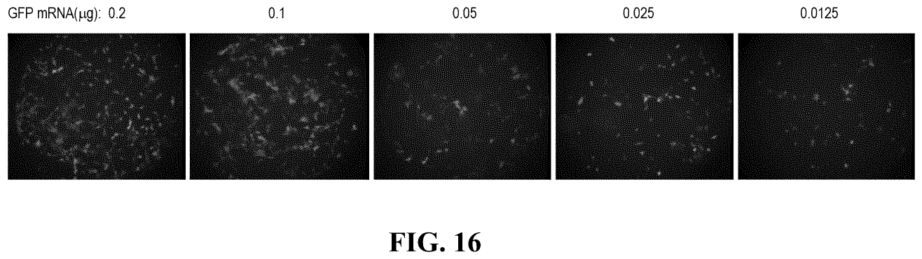

HEK293 cells were transfected using CPT153E LNP loaded with 0.0125-0.2 μg GFP mRNA, and the fluorescence images were recorded after 24 hours. As shown in FIG. 16, all the transfected cells emitted bright green fluorescent signals of GFP, indicating that not only transiently charged cationic lipids, but also permanently charged cationic lipids, can be used to generate PALNPs.

### Example 14. LNP with Reversed Triblock Co-Polymer PPOₓ-PEO_{y}-PPOₓ

The block structure of the co-polymer can also be reversed i.e., one PEO block can be located in the middle of the tri-blocks, and two PPO blocks can be located flanking the PEO block. One exemplary structure of the inverted co-polymer is shown below.

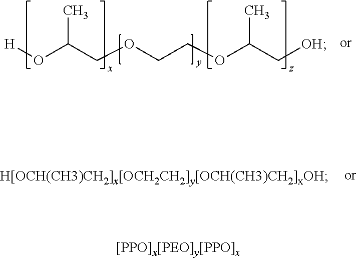

Specifically, L31R1 (x=25.6, y=7.5) has an average molecular weight of about 3300 Daltons, with the weight percentage of PEO of about 10%; L17R4 (x=14, y=24.5) has an average molecular weight of about 2700 Daltons, with the weight percentage of PEO of about 40%.

CPT189 LNP and CT202 LNP were generated using the method described herein, and their formulations are listed in the table below.

TABLE 11
The formulations of LNP CPT189 and CPT202

| | ALC-0315 | DSPC | DOPE | Cholesterol | PEG2000-DSPE | L31R1 | L17R4 | N/P |
|---|---|---|---|---|---|---|---|---|
| CPT189 | 21.7 | 16.9 | 16.9 | 41.0 | 1.2 | 2.4 | | 1 |
| CPT202 | 21.7 | 16.9 | 16.9 | 41.0 | 1.2 | | 2.4 | 1 |

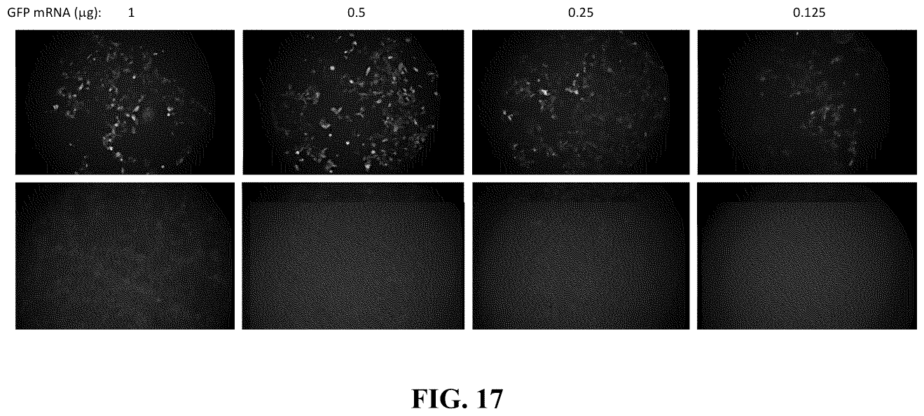

HEK293 cells were transfected using CPT189 LNP loaded with 0.0125-1 μg GFP mRNA, and the fluorescence images were recorded after 24 hours. As shown in FIG. 17, all the transfected cells emitted bright green fluorescence of GFP, indicating that co-polymers having an inverted structure can also be used to generate PALNPs. The results also show that the transfection efficiency of CT202 is significantly weaker than that of CPT189, although the molecular weight of the two polymers are very close. The only difference of the two PALNPs is the PEO:PPO ratio. In particular, the weight percentage of PEO in CPT189 is 10%, as compared to 40% in L17R4. The results indicate that lower PEO content favors mRNA transfection.

### Example 15. Optimization of the Molar Ratio of Triblock Co-Polymers

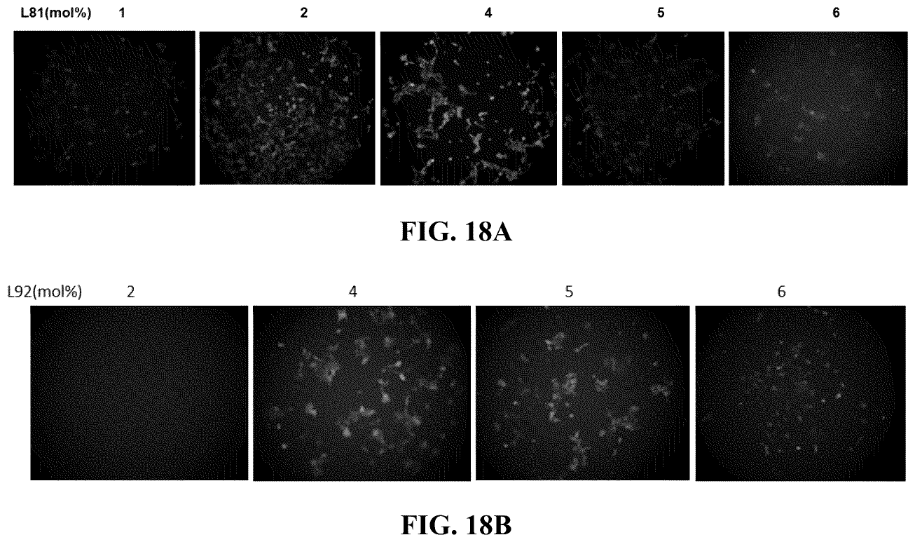

The optimized molar ratios of the triblock co-polymers were identified using in vitro tests. As shown in FIGS. 18A-18B, the base LNP formulation was fixed to contain 18% ALC-0315, 20% DSPC, 20% DOPE, 34% cholesterol, 1.5% PEG2000-DEPE, and less than 10% (e.g., 1%, 2%, 4%, 5%, or 6%) triblock co-polymer L81 or L92. The results indicate that the optimized molar ratio is about 2-4% for L81, about 4-5% for L92, and about 5-7% for L121.

### Example 16. The PALNPs are Non-Cytotoxic

The cationic or ionizable lipids, RNA and DNAs are all immunostimulants. For example, a low dose of cationic lipids and/or oligo nucleotides can be used as adjuvants in vaccines to stimulate the innate immune responses. Thus, a combination of cationic lipids and RNA/DNAs in nanoparticles may induce strong innate immune responses and cause severe adverse reactions to the patients.

Indeed, cationic and ionizable lipids have been reported to stimulate the secretion of pro-inflammatory cytokines and reactive oxygen species, although the molecular mechanisms for the immunogenicity of these lipids have not yet been fully understood. Cytotoxicity of lipid materials is also a safety concern, depending on the dose, lipid properties and cell types. In vivo application of lipid nanoparticles has been reported to induce liver and lung injuries in rodents, which may be attributed to the cytotoxicity of the materials and the induction of pro-inflammatory factors. The toxicity of LNP badly limited its applications in gene vaccines and gene therapeutics.

As shown in the results described above for the PALNPs, when the ratio of the ionizable (cationic) lipids to mRNA (DNA), or the N/P ratio was reduced 6-24 times, the mRNA/DNA transfection efficiency of the LNP was dramatically increased compared to the reference LNPs. Combining these two dramatic improvements of the PALNP, when the mRNA dose is decreased by one-fold and the dose of ionizable lipid is decreased by 6-24 times, the ionizable (cationic) lipids used can be decreased for hundreds of times. Thus, the PALNPs can eliminate or minimize the toxicities of LNPs, and promote its applications in genetic vaccines, genetic therapeutics and gene editing.

The cationic lipids can also disrupt the cell membranes as well as the endosome and lysosomes of the cells. Actually, endosome/lysosome membrane disruption by the cationic lipids plays an key role in LNP escaping from the lysosome for mRNA/DNA delivery.

However, the disruption of the lysosomes is a double-edged sword. For example, the process can also release cytotoxic enzymes into the cytosol, e.g., proteases, nucleases, phosphatases, sulfatases, and lipid degrading enzymes. These enzymes can damage the normal cell organelles, causing acute or apoptotic cell death, and further inducing systemic adverse reactions. Details can be found, e.g., in Forster III, J., et al. “mRNA-carrying lipid nanoparticles that induce lysosomal rupture activate NLRP3 inflammasome and reduce mRNA transfection efficiency.” Biomaterials Science 10.19 (2022): 5566-5582.

In addition, as the toxicity is cationic lipid dose-dependent, the PALNPs disclosed herein with very low dose of cationic lipid and enhanced gene transfection efficiency can solve the long-lasting toxicity problems of LNPs. To demonstrate this extraordinary potential of the PALNPs, the HEK293 cells were transfected with CPT147P LNP and the corresponding reference LNP CPT107. Similarly, cell transfection was performed using CPT149E LNP and the corresponding reference LNP CPT109. The formulations of these LNPs are listed in the table below.

TABLE 12
The formulations (mol %)of LNPs used in the cytotoxicity study.

| | | | | | | PEG2000- | | |
|---|---|---|---|---|---|---|---|---|
| | ALC-0315 | SM-102 | DSPC | DOPE | Cholesterol | DSPE | L121 | N/P |
| CPT147P | 18.1 | | 18.1 | 18.1 | 39.2 | 1.5 | 5 | 1 |
| CPT107 | 50 | | 10 | | 38.5 | 1.5 | | 6 |
| CPT149E | | 18 | 19.8 | 19.8 | 34.1 | 1.5 | 6.8 | 1 |
| CPT109 | | 50 | 10 | | 38.5 | 1.5 | | 6 |

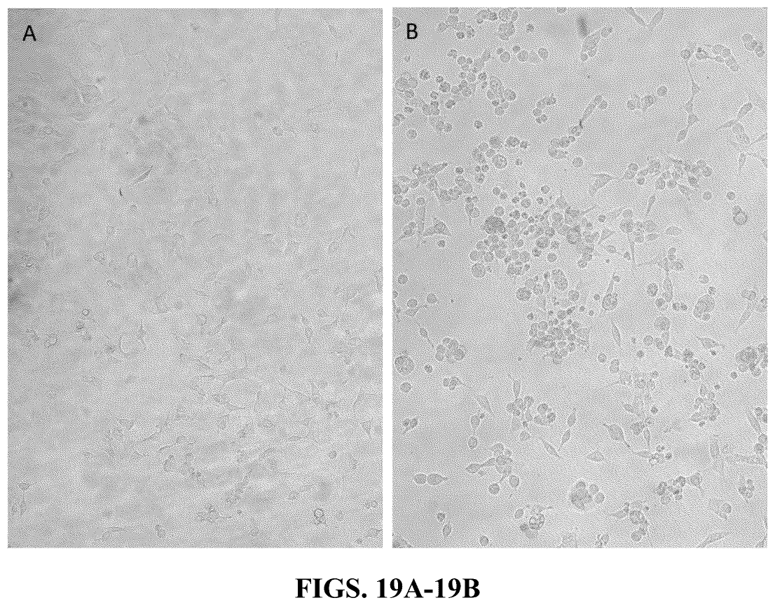

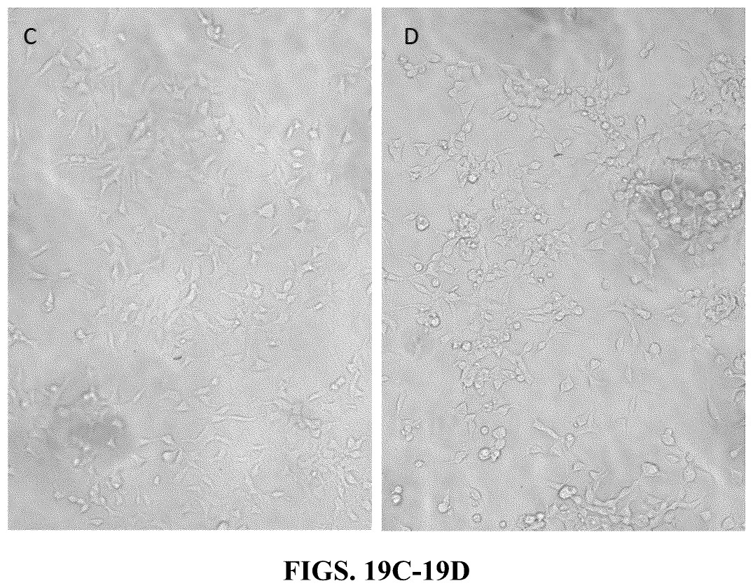

All the LNPs were added to the cells in a well of a 96-well plate, which contained 0.2 μg GFP mRNA. As shown in FIGS. 19A-19D, the bright field images of the cells were taken 24 hours after the addition of the LNPs to the cells.

The images showed that the cells exposed to the PALNPs (CPT147P and CPT149E) exhibited a healthy morphology. In contrast, many cells exposed to the reference LNPs (CPT107 and CPT109) were rounded up, or detached from the bottom of the well, indicating extensive apoptosis occurring to the cells. The results demonstrate that the PALNPs can reduce or eliminate the cytotoxicity, and have great application potentials in the development of safe and effective genetic vaccines and genetic therapeutics.

### Example 17. In Vivo Distribution and Improved Expression of mRNA Delivered by PALNPs

Several animal studies were performed to evaluate the in vivo mRNA transfection functions. Specifically, BALB/c mice were administrated with the LNP CPT147E-10, which shares the same formulations with CPT147E-05 in Table 7, and the corresponding reference LNP CPT107. The LNPs carrying luciferase reporter mRNA were administered either via tail vein injection or intramuscular injection. At the selected time points (4-24 hours post injection), the mice were anesthetized by isoflurane and dosed with the substrate luciferin. The body images were taken by measuring the photon intensity of bioluminescence, which indicated the expression level of the luc mRNA in the mice body as well as the mRNA distributions in the organs.

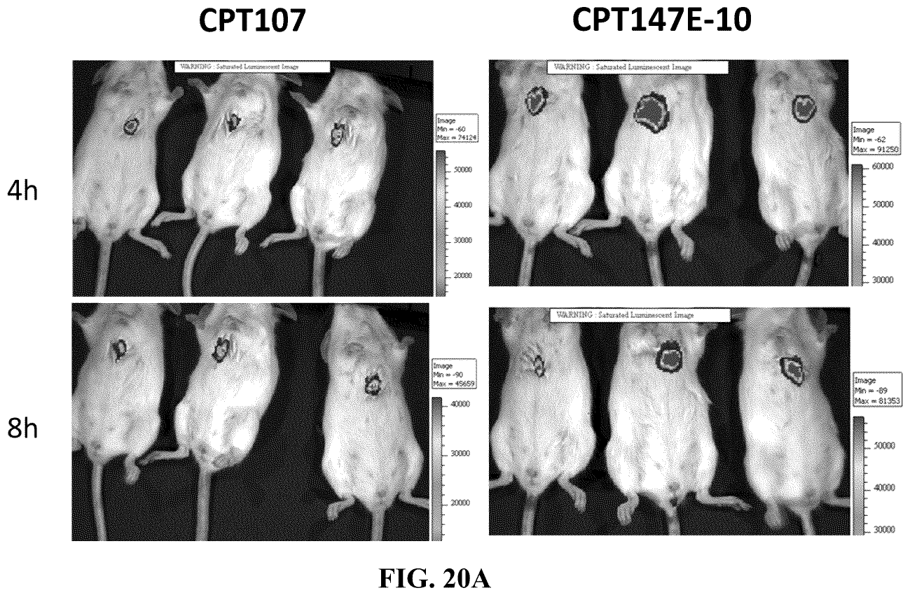

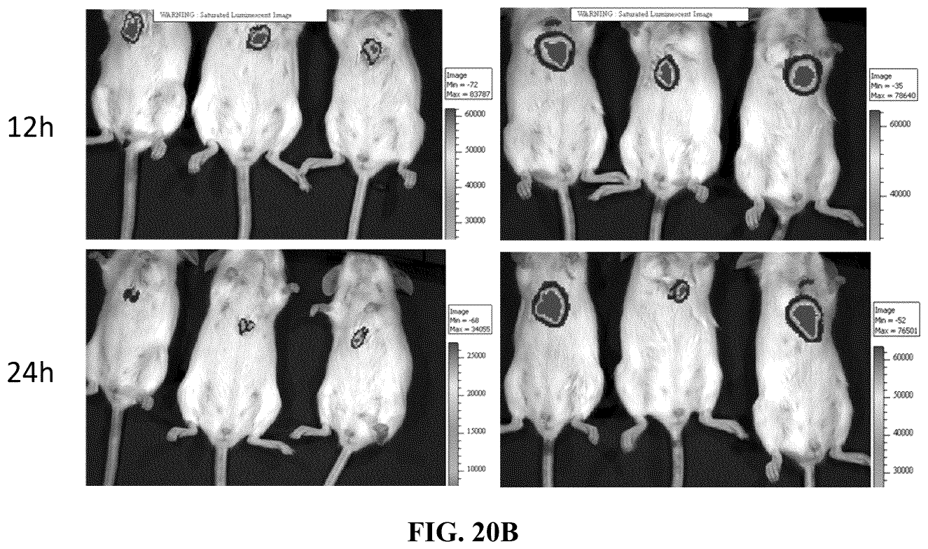

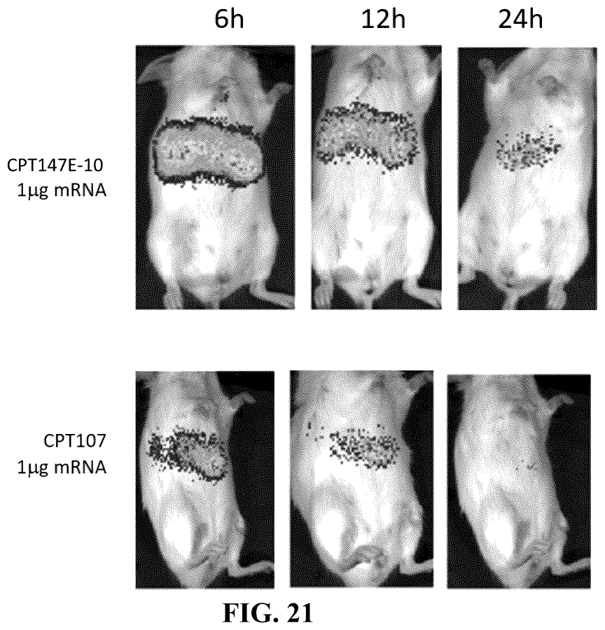

As shown in FIGS. 20A-20B, CPT147E-10 LNP significantly enhanced the in vivo mRNA expression as compared to CPT107, when the LNPs were loaded with 10 μg mRNA. For example, the mice injected with CPT147 exhibited brighter photon intensities than mice injected with CPT107 throughout the experimental period. In another experiment when LNPs were loaded with 1 μg mRNA, the mice injected with CPT147E-10 also exhibited brighter photon intensities than mice injected with CPT107 (FIG. 21).

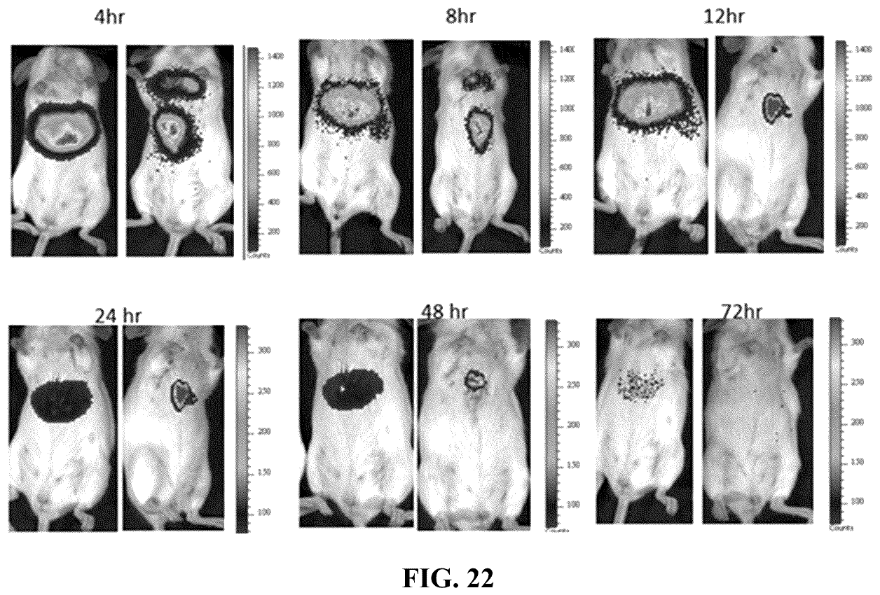

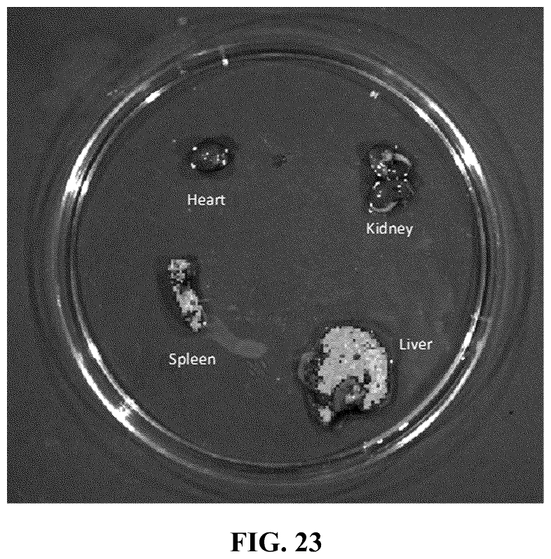

In a different experiment, the PALNP CPT147E-10 and the corresponding reference LNP CPT107 were injected via the mouse tail veins, and the body images of the mice were taken for 4-72 hours. As shown in FIG. 22, the mice injected with CPT147 exhibited brighter photon intensities than mice injected with CPT107 throughout the experimental period. The results also indicate that the mRNA delivered by CPT147E-10 maintained a long-lasting protein expression, e.g., for at least 72 hours. Further, the mouse was sacrificed 6 hours after it was injected with CPT147E-10 containing 10 μg mRNA, and the bioluminescence images of the organs including heart, kidney, spleen, and liver were isolated. As shown in FIG. 23, strong bioluminescence was seen from the liver and spleen. There was no detectable luminescence observed from the heart and kidney.

### Example 18. The PALNPs Prevent mRNA from Unwanted Systemic Delivery Via Intramuscular Administration

Intramuscular administration route is applied for injecting COVID-19 mRNA vaccines. While the targeted distribution organ of the mRNA is the muscle cells at injection site, the actual distribution in the human body is currently unknown. Among the various adverse reactions were liver injuries caused by COVID-19 mRNA vaccines. Moreover, the kidney delivery of the mRNA vaccine was attributed to the life-threatening myocarditis. Details can be found, e.g., in Mann, R., et al. “Drug-induced liver injury after COVID-19 vaccine.” Cureus 13.7 (2021); and Cadegiani, F. A., “Catecholamines Are the Key Trigger of COVID-19 mRNA Vaccine-Induced Myocarditis: A Compelling Hypothesis Supported by Epidemiological, Anatomopathological, Molecular, and Physiological Findings.” Cureus 14.8 (2022). Indeed, it was quite common that after the injection of mRNA LNPs, besides the local injection site, extensive mRNA expression was also observed in the liver and other organs. To prevent the intramuscularly administrated mRNA from entering the systemic delivery is clinically critical and technically challenging. Surprisingly, CPT147 LNP provided the solution to this challenging problem.

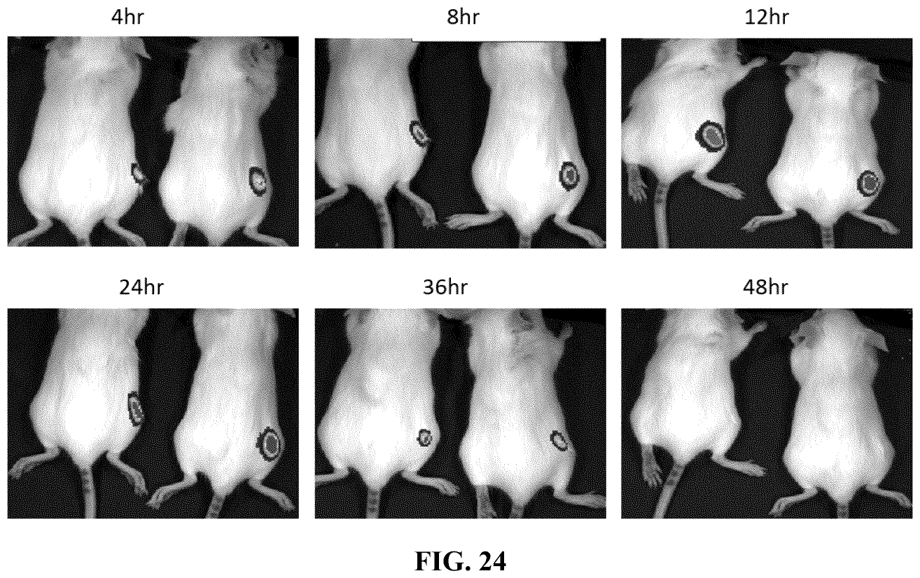

FIG. 24 shows the results of BALB/c mice administered with the PALNP CPT147 by intramuscular injection. Specifically, the mice were injected with 10 μg luc mRNA encapsulated in CPT147. Strong protein expression was detected only from the injection site, and no bioluminescence was observed from the supine positions. It is contemplated that the reason for the ideally restricted local delivery is that the LNP was quickly uptaken by the muscle cells at the injection site, in a manner similar to the quick cell uptake firmly observed in the in vitro studies described above, which left little chance for systemic delivery.

### Example 19. Preparation of mRNA/DNA Lipid Nanoparticles

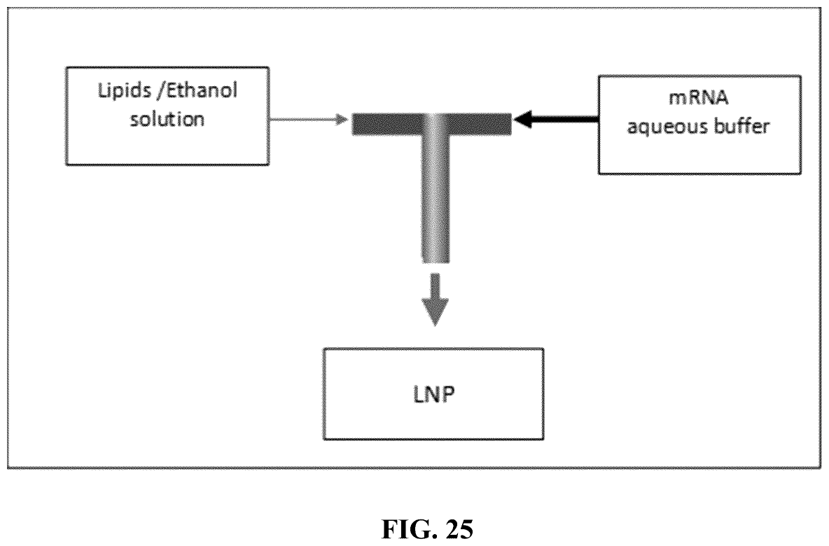

Currently, the LNP preparations for the pharmaceutical research and pharmaceutical commercial products utilize the T- or Y-mixing setups, as illustrated in FIG. 25. In this process, the lipid solution in ethanol can be fed by a pump into the T junction from one arm of the T mixer, and the mRNA aqueous solution can be fed by another pump in the opposite arm of the T mixer. The volume ratios of the aqueous flow to the organic flow are in the range of 3-10. The mixed solution can flow out the T junction via a channel from the T mixer, and the LNP is formed in the mixed solution via self-assembly. The quality of the LNP depends on how sufficient the mixing of the two solutions is. For example, when the assembling process occurs in a homogenous solution, the nanoparticles can form with homogenous particle size and morphology. However, when the assembling process occurs in a partially mixed solution, metastable LNP can form with heterogenous compositions, particle sizes, and morphologies. The commonly observed blebbing problems of the LNPs made by the T mixer could be associated with the imbalanced feeding, which can cause insufficient mixing of the lipid solution and the aqueous solution in the T mixing process.

All the LNPs described herein were prepared using the nPort™ technology disclosed in U.S. Pat. No. 11,497,715B2 and U.S. Pat. No. 9,693,958B2. In this process, a 5-6 ports of stainless steel mixing device was used for mixing the lipids/ethanol solution with the aqueous solution(s) in the presence or absence of mRNA/DNA. The organic stream was pumped into the mixing chamber via one inlet port, and the aqueous solution was evenly fed via multiple inlet ports. The flow streams were rapidly and homogeneously mixed in the mixing chamber in a time scale of about sub-milliseconds. The mixed solution flow away through the outlet port(s) and was collected. The LNP self-assembly proceeded in the solution. The sizes of the inlet(s), the outlet(s), the mixing chamber, and the flow rates of the solutions were all rationally designed to ensure the complete mixing of the solutions at various scales of the preparations. In some preparations, two or more aqueous solutions were applied to meet with the chemical and physicochemical properties of the LNP components.

The residual ethanol was removed from the product, and buffer exchange was performed by either dialysis (for samples with a small volume) or by tangent flow filtration (TFF). The ethanol removal from some small samples was also performed with vacuum in a ventilation hood.

When mRNA/DNA was included in the aqueous buffer, the LNP obtained through the process above was the entire LNP, when the residual ethanol is removed from the mixture by evaporation, or dialysis, or tangent flow filtration, the LNP is finalized. When mRNA/DNA was not included in the aqueous buffer, the LNP obtained through the process above was the blank LNP. The mRNA-loaded LNP were made by mixing the blank LNP suspension and mRNA solution by vortexing or stirring. An exemplary process for preparing LNP CPT163-012 is shown below.

### Preparation of CPT163-012

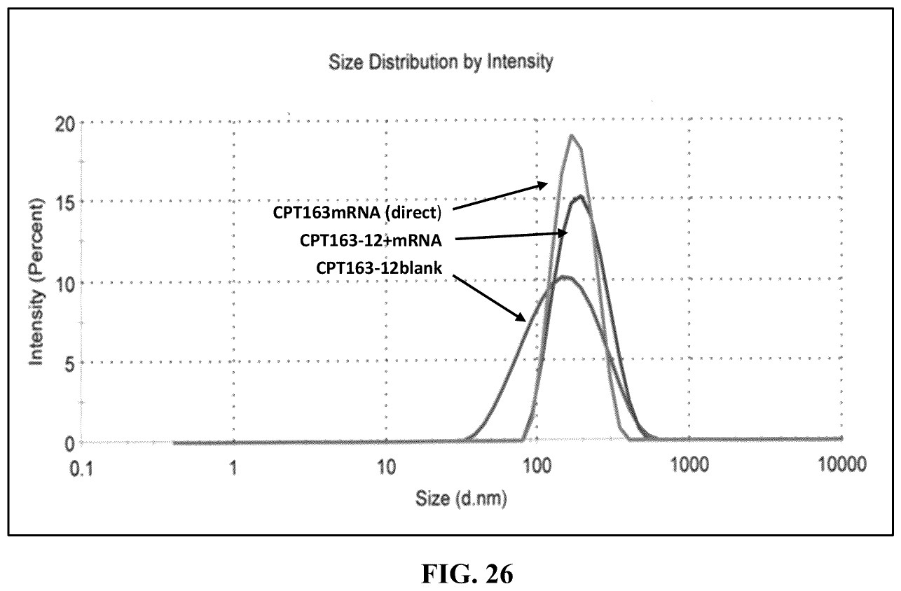

1) lipids/ethanol solution: lipids/anhydrase ethanol solution containing 6.00 mg ALC-0315, 6.75 mg DSPC, 6.41 mg DOPE, 5.76 mg cholesterol. 2 15 mg L81, and 1.48 mg PEG2000-DSPE.
2) Aqueous buffer: 5 mM citrate, 75 mM NaCl, pH
3) Mixing process. 1 mL of lipids/ethanol solution was loaded into a 1 ml syringe driven by one syringe pump; and 4.4 mL citrate buffer was evenly split into 3 syringes driven by another syringe pump. The syringe was connected to an inlet port of a 5-port device. The port size (diameter) and the mixing chamber diameter were 0.25 mm; each syringe was connected to an inlet port by a tubing. The diameter and the length of the outlet tubing mounted to the outlet port were 0.17 mm and 60 mm, respectively. The lipid and the aqueous buffer loaded in the syringes were pumped into the mixing chamber of by a syringe pump with a flow rate of 8 mL/min in total. The LNP solution exited through the outlet tubing and was collected in a glass vial. After the residual ethanol was removed from the solution by vacuum, the blank LNP (without mRNA/DNA) were obtained. The lipid concentrations were determined by HPLC.
4) Loading of mRNA. The final mRNA LNP was obtained by a simple mix of mRNA solution with the blank LNP. For example, 20.5 μl of the black LNP that contains 55.4 μg of lipids (including L81 and 11.71 μg of ALC-0315), was placed into a 0.5 mL Eppendorf tube and in 5 μl of mRNA solution containing 5 μg of green fluorescence protein mRNA was added by pipetting followed by gentle vortexing. The mixture was then incubated at 37° C. for 10 minutes. The final mRNA LNP was obtained. The N/P ratio of this preparation was 1. The w/w ratio of the total lipids:mRNA was 11:1, and the w/w of ionizable ALC-0315 to mRNA was 2.322:1. Similarly, LNP with N/P=0.5 can be made by mixing 20.5 μl of the black LNP with 10 μg of mRNA; and LNP with N/P=2 can be made by mixing 20.5 μl of the black LNP with 2.5 μg of mRNA.
5) Characterization of LNP. The encapsulation percentage of mRNA/DNA was measured by the Ribogreen method. Details can be found, e.g., in Naderi S. A., et al. “Development of an mma-lnp vaccine against sars-Cov-2: Evaluation of immune response in mouse and rhesus macaque.” Vaccines 9.9 (2021): 1007. The encapsulation percentage was in the range of 75-95%. The particle size of the blank LNP and mRNA-loaded LNP were determined by dynamic light scattering method (Nano Zetasizer, Malvern). The particle size distributions of CPT163-12 are shown in FIG. 26. The particle size and polydispersity were 128.3 nm and 0.220 for the blank LNP, and 180.1 nm and 0.125 for the mRNA-loaded LNP. The dramatic change in the particle size between blank LNP and the mRNA-loaded LNP, and the high homogeneity of the mRNA-loaded LNP indicate a reformation of the LNP upon the mixing of the blank LNP with mRNA, instead of an disordered aggregation of the LNP. In addition, the excellent mRNA encapsulation percentage and the high mRNA transfection efficiency demonstrate that mRNA was encapsulated inside the reformed LNP rather than adsorbed to the surface of the blank LNP.
6) Direct mixing of mRNA with lipids/ethanol solution. The mRNA-loaded LNP could be made by directly mixing the lipids/ethanol solution with the aqueous solution of mRNA. For example, 0.5 mL of lipids/ethanol solution can be loaded into a 1 mL air-tight glass syringe containing 0.5 mg ALC-0315 ionizable lipid, 0.563 mg DSPC, 0.533 mg DOPE, 0.480 mg cholesterol, 0.179 mg L81, and 0.124 mg PEG2000-DSPE. 2.2 mL mRNA solution containing 0.215 mg mRNA (5 mM citrate, 75 mM NaCl, pH 5.5) was evenly split into 3 syringes driven by another syringe pump. The syringe was connected to an inlet port of a 5-port device. The port size (diameter) and the mixing chamber diameter were 0.25 mm; each syringe was connected to an inlet port by a tubing. The diameter and the length of the outlet tubing mounted to the outlet port were 0.17 mm and 60 mm, respectively. The lipids and the aqueous buffer loaded in the syringes were pumped into the mixing chamber by a syringe pump with a flow rate of 8 mL/min in total. The LNP solution exited through the outlet tubing and was collected in a glass vial. The residual ethanol was removed from the solution by vacuum. The N/P ratio of the LNP was 1. The w/w ratio of total lipids:mRNA was 11:1. The particle size was 164.7 nm, and the polydispersity was 0.075. The particle size distribution curve is shown in FIG. 26.

### Example 20. LNP with Polymer Propylene Glycol (PPO)

The co-polymers used to generate PALNPs were also replaced with polymer propylene glycol (PPO). One exemplary structure of the poly-PPO is shown below

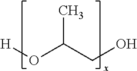

Specifically, the poly-PPO (x=46.5) has an average molecular weight of about 2700 Daltons (PPO₂₇₀₀). CPT201 LNP were generated using the method described herein, and the formulation is listed in the table below.

TABLE 13
The formulation of LNP CPT201

| | ALC-0315 | DSPC | DOPE | Cholesterol | PEG2000-DSPE | PPO2700 | N/P |
|---|---|---|---|---|---|---|---|
| CPT201 | 22.5 | 15.7 | 15.7 | 42.7 | 1.1 | 2.2 | 1 |

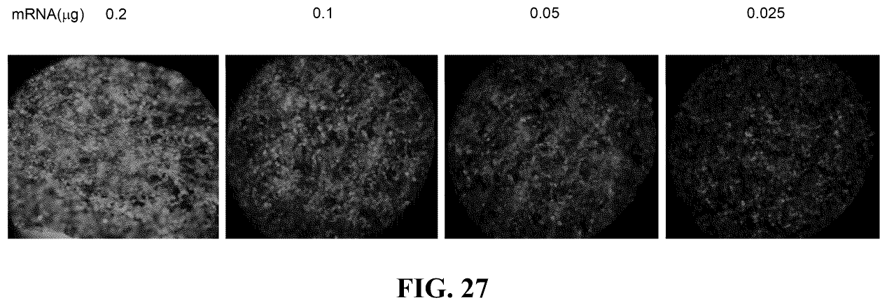

HEK293 cells were transfected using CPT201 LNP loaded with 0.0125-0.2 μg GFP mRNA, and the fluorescence images were recorded after 24 hours. As shown in FIG. 27, all the transfected cells emitted bright green fluorescent signals of GFP, indicating that poly-PPO can be used to generate PALNPs.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A nanoparticle composition comprising
a lipid component, and
a polymer component,
wherein the polymer component comprises:
(a) a compound of formula I (PEOₓ-PPO_{y}-PEO_{z}) 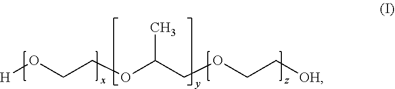
wherein x is a number selected from 1-100, y is a number selected from 10-500, and z is a number selected from 1-100;
(b) a compound of formula II (PPOₓ-PEO_{y}-PPO_{z}) 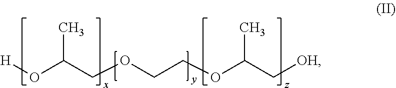
wherein x is a number selected from 10-500, y is a number selected from 1-100, and z is a number selected from 10-500;
(c) a compound of formula II (PEO_{y}-PPO_{z}) 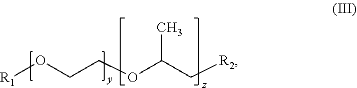
wherein R1 is H or CH₃, R₂ is OH or OCH₃, y is a number selected from 1-100, and z is a number selected from 5-500; and/or
(d) a compound of formula IV (PPOₓ) 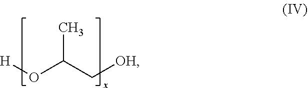
wherein x is a number selected from 1-100.

2. The nanoparticle composition of claim 1, wherein the polymer component accounts for about 0.1 mol % to about 20 mol % of the nanoparticle composition.

3. The nanoparticle composition of claim 1 or 2, wherein the lipid component comprises
an ionizable and/or permanently charged cationic lipid,
a helper lipid,
a structural lipid, and/or
a PEG (polyethylene glycol) lipid.

4. The nanoparticle composition of claim 3, wherein the ionizable and/or permanently charged cationic lipid accounts for about 5 mol % to about 30 mol % of the nanoparticle composition.

5. The nanoparticle composition of claim 3 or 4, wherein the helper lipid is a phospholipid.

6. The nanoparticle composition of any one of claims 3-5, wherein the helper lipid accounts for about 10 mol % to about 50 mol % of the nanoparticle composition.

7. The nanoparticle composition of any one of claims 3-6, wherein the structural lipid is cholesterol.

8. The nanoparticle composition of any one of claims 3-7, wherein the structural lipid accounts for about 20 mol % to about 50 mol % of the nanoparticle composition.

9. The nanoparticle composition of any one of claims 3-8, wherein the PEG lipid has an average molecular weight of about 500-5000 Daltons (e.g., about 2000 Daltons).

10. The nanoparticle composition of any one of claims 3-9, wherein the PEG lipid accounts for about 0.5 mol % to about 5 mol % of the nanoparticle composition.

11. The nanoparticle composition of any one of claims 1-10, comprising:
(a) about 1 mol % to about 20 mol % of the compound of formula I;
(b) about 5 mol % to about 30 mol % of the ionizable and/or permanently charged cationic lipid (e.g., ALC-0315, SM-102, DLin-DMA, DLin-MC3-DMA, and/or DLin-KC2-DMA);
(c) about 10 mol % to about 50 mol % of the helper lipid (e.g., DSPC and/or DOPE);
(d) about 20 mol % to about 50 mol % of the structural lipid (e.g., cholesterol); and
(e) about 0.5 mol % to about 5 mol % of the PEG lipid (e.g., PEG2000-DSPE).

12. The nanoparticle composition of claim 11, wherein the number x and number z are identical in formula I.

13. The nanoparticle composition of claim 11 or 12, wherein the helper lipid comprises about 5 mol % to about 30 mol % of DSPC, and/or about 5 mol % to about 30 mol % of DOPE.

14. The nanoparticle composition of any one of claims 11-13, wherein the compound of formula I has an average molecular weight of about 1000 Daltons to about 30000 Daltons (e.g., about 1000 Daltons to about 10000 Daltons).

15. The nanoparticle composition of any one of claims 11-14, wherein the number x is selected from 1-15, the number y is selected from 30-80, and the number z is selected from 1-15.

16. The nanoparticle composition of any one of claims 11-15, wherein the compound of formula I is L121, L92, or L81.

17. The nanoparticle composition of any one of claims 1-10, comprising:
(a) about 1 mol % to about 20 mol % of the compound of formula II;
(b) about 5 mol % to about 30 mol % of the ionizable and/or permanently charged cationic lipid (e.g., ALC-0315, SM-102, DLin-DMA, DLin-MC3-DMA, and/or DLin-KC2-DMA);
(c) about 10 mol % to about 50 mol % of the helper lipid (e.g., DSPC and/or DOPE);
(d) about 20 mol % to about 50 mol % of the structural lipid (e.g., cholesterol); and
(e) about 0.5 mol % to about 5 mol % of the PEG lipid (e.g., PEG2000-DSPE).

18. The nanoparticle composition of claim 17, wherein the number x and number z are identical in formula I.

19. The nanoparticle composition of claim 17 or 18, wherein the helper lipid comprises about 5 mol % to about 30 mol % of DSPC, and/or about 5 mol % to about 30 mol % of DOPE.

20. The nanoparticle composition of any one of claims 17-19, wherein the compound of formula II has an average molecular weight of about 1000 Daltons to about 30000 Daltons (e.g., about 1000 Daltons to about 10000 Daltons).

21. The nanoparticle composition of any one of claims 17-20, wherein the number x is selected from 10-50, the number y is selected from 1-30, and the number z is selected from 10-50.

22. The nanoparticle composition of any one of claims 17-21, wherein the compound of formula II is L31R1 or L17R4.

23. The nanoparticle composition of any one of claims 1-10, comprising:
(a) about 1 mol % to about 20 mol % of the compound of formula IV;
(b) about 5 mol % to about 30 mol % of the ionizable and/or permanently charged cationic lipid (e.g., ALC-0315, SM-102, DLin-DMA, DLin-MC3-DMA, and/or DLin-KC2-DMA);
(c) about 10 mol % to about 50 mol % of the helper lipid (e.g., DSPC and/or DOPE);
(d) about 20 mol % to about 50 mol % of the structural lipid (e.g., cholesterol); and
(e) about 0.5 mol % to about 5 mol % of the PEG lipid (e.g., PEG2000-DSPE).

24. The nanoparticle composition of claim 23, wherein the helper lipid comprises about 5 mol % to about 30 mol % of DSPC, and/or about 5 mol % to about 30 mol % of DOPE.

25. The nanoparticle composition of claim 23 or 24, wherein the compound of formula IV has an average molecular weight of about 1000 Daltons to about 30000 Daltons (e.g., about 1000 Daltons to about 10000 Daltons).

26. The nanoparticle composition of any one of claims 23-25, wherein number x in the compound of formula IV is about 30 to about 60.

27. The nanoparticle composition of any one of claims 23-26, wherein the compound of formula IV is PPO₂₇₀₀.

28. A lipid nanoparticle (LNP) having the nanoparticle composition of any one of claims 1-27.

29. The LNP of claim 28, further comprising a nucleic acid. wherein the nucleic acid comprises a DNA (e.g., double stranded DNA (dsDNA), plasmid DNA, single stranded DNA (ssDNA), or an antisense DNA thereof) or an RNA (e.g., small interfering RNA (siRNA), microRNA (miRNA), messenger mRNA (mRNA), guide RNA (gRNA), circular RNA (circRNA), self-amplifying RNA (saRNA), or an antisense RNA thereof).

30. The LNP of claim 28 or 29, wherein the N/P ratio of the nanoparticle composition is from about 0.1 to about 10 (e.g., about 0.1 to about 5, about 0.1 to about 4, about 0.1 to about 3, about 0.2 to about 2, or about 0.2 to about 1.5).

31. The LNP of any one of claims 28-30, wherein the compound of formula I, formula II, formula III, formula IV, and/or formula V has a hydrophilic lipophilic balance (HLB) value from 1 to 18.

32. The LNP of any one of claims 28-31, wherein the mean size of the LNP is from about 30 nm to about 2000 nm (e.g., about 30 nm to about 1000 nm, or about 30 nm to about 500 nm).

33. The LNP of any one of claims 28-32, wherein the polydispersity index of the LNP is from about 0.001 to about 0.5 (e.g., from about 0.01 to about 0.3).

34. The LNP of any one of claims 28-33, wherein the LNP has a zeta potential of about −30 mV to about +20 mV.

35. The LNP of any one of claims 29-34, wherein the w/w ratio of the lipid component to the nucleic acid is from about 2:1 to about 50:1 (e.g., from about 2:1 to about 20:1).

36. The LNP of any one of claims 29-35, wherein the encapsulation efficiency of the nucleic acid is at least 50%, at least 60%, at least 70%, at least 80%, or at least 90%.

37. A method of delivering a nucleic acid to a mammalian cell, wherein the method comprises administering the LNP of any one of claims 28-36 to a subject, wherein the administering comprises contacting the mammalian cell with the nanoparticle composition, whereby the nucleic acid is delivered to the mammalian cell.

38. The method of claim 37, wherein the mammalian cell is in a mammal.

39. The method of claim 37 or 38, wherein the LNP is associated with a therapeutic medicine, a vaccine (e.g., a prophylactic vaccine or a therapeutic vaccine), gene editing, or cell-based therapies (e.g., chimeric antigen receptor (CAR)-T therapies).

40. The method of claim 37 or 38, wherein the LNP is associated with treatment of a disease (e.g., infectious disease, autoimmune disease, cancers, or genetic disorders).

41. The method of any one of claims 37-40, wherein the LNP is delivered by mouth, nasal, dermal, vein, topical, ophthalmic, and/or mucosal, intradermal, and intramuscular administration.

42. A method for the enhanced delivery of a nucleic acid to a target tissue, wherein the method comprises
administering the LNP of any one of claims 28-36 to a subject, wherein the administering comprises contacting the target tissue with the LNP, whereby the nucleic acid is delivered to the target tissue.

43. A method of producing a polypeptide of interest in a mammalian cell, said method comprising
administering the LNP of any one of claims 29-36 to a subject, wherein the nucleic acid encodes the polypeptide of interest, whereby the nucleic acid is capable of being translated in the mammalian cell to produce the polypeptide of interest.

44. A method of making a LNP having a nanoparticle composition comprising a lipid component and a polymer component, wherein the lipid component comprises: an ionizable and/or permanently charged cationic lipid, a helper lipid, a structural lipid, and a PEG (polyethylene glycol) lipid, wherein the polymer component comprises: a compound of formula I, formula II, formula III, and/or formula IV,
wherein the method comprises:
(a) introducing one or more streams of a lipid solution in a water-miscible organic solvent via a first set of one or more inlet ports connected to a mixing chamber, wherein the lipid solution comprises the lipid component and the polymer component,
(b) introducing one or more streams of an aqueous solution via a second set of one or more inlet ports connected to the mixing chamber,
(c) mixing the one or more streams of the lipid solution and the one or more streams of the aqueous solution in a mixing chamber to generated the LNP, and
(d) recovering the LNP via one or more outlet ports connected to the mixing chamber.

45. The method of claim 44, wherein the aqueous solution comprises a nucleic acid.

46. The method of claim 44 or 45, wherein the angle between any of the first set of one or more inlet ports and any of the second set of one or more inlet ports is 0-180 degrees.
